(19) United States
(12) Patent Application Publication
(10) Pub. No.: US 2022/0298508 A1
(43) Pub. Date: Sep. 22, 2022

(54) GALNAC CLUSTER PHOSPHORAMIDITE AND TARGETED THERAPEUTIC NUCLEOSIDES

(71) Applicant: Nanjing GeneLeap Biotechnology Co., Ltd., Nanjing (CN)

(72) Inventors: Jason Zhang, Walpole, MA (US); Ekambareswara Kandimalla, Hopkinton, MA (US); Jun Jiang, Westwood, MA (US); Sheng Bi, Nanjing (CN); Pengfei Li, Nanjing (CN); Xin Xu, Nanjing (CN); Lakshmi Bhagat, Framingham, MA (US)

(21) Appl. No.: 17/689,895

(22) Filed: Mar. 8, 2022

Related U.S. Application Data
(60) Provisional application No. 63/158,338, filed on Mar. 8, 2021.

(51) Int. Cl. C12N 15/113; A61K 47/54; A61K 31/713

(52) U.S. Cl. CPC C12N 15/113; A61K 47/549; A61K 31/713; C12N 2310/351; C12N 2310/14; C12N 2310/315

(57) ABSTRACT

Provided herein are oligonucleotide agents comprising one or more therapeutic oligonucleotides such as siRNA and one or more targeting conjugate compounds. In certain embodiments, the conjugate compound comprises one or more N-Acetylgalactosamine as targeting group, branching group and linker group. By incorporating long carbon chains into the GalNAc clusters instead of using multiple amide groups to elongate the chain length, simplification of the synthesis by reducing the number of steps is achieved.

### STATEMENT REGARDING THE SEQUENCE LISTING

The Sequence Listing associated with this application is provided in text format in lieu of a paper copy, and is hereby incorporated by reference into the specification. The name of the text file containing the Sequence Listing is 690229_401_SEQUENCE LISTING.txt. The text file is 51 KB, was created on May 16, 2022, and is being submitted electronically via EFS-Web.

### TECHNICAL FIELD

The present invention relates to the field of therapeutic agent delivery using carbohydrate conjugates. In particular, the present invention provides novel carbohydrate conjugates and iRNA agents comprising these conjugates, which are advantageous for the in vivo delivery of these iRNA agents, as well as iRNA compositions suitable for in vivo therapeutic use. Additionally, the present invention provides methods of making these compositions, as well as methods of introducing these iRNA agents into cells using these compositions, e.g., for the treatment of various disease conditions, including metabolic diseases or disorders, such as hepatic diseases or disorders.

### BACKGROUND

Targeted delivery of therapeutic agents to hepatocytes is a particularly attractive strategy for the treatment of metabolic, cardiovascular and other liver diseases. The asialoglycoprotein receptor (ASGP-R) is abundantly expressed on hepatocytes and minimally found on extra-hepatic cells, making it an ideal entry gateway for hepatocyte-targeted therapy. The carbohydrate binding domain for ASGPR has been elucidated, making the design of effective binders more straightforward (Bioconjugate Chem. 2017, 28, 283-295). Numerous multivalent ligands have been developed to target ASGP-R, among which well-defined multivalent N-acetyl D galactosamine (GalNAc) moieties display high binding affinity (J Am Chem Soc. 2017, 139, 3528-3536). Recently, several gene delivery systems based on GalNAc ligand for ASGP-R showed encouraging clinical results and the FDA has approved siRNAs conjugated to GalNAc for liver diseases (Molecular Therapy, 2020, 28, 1759-1771).

Antisense oligonucleotides (ASOs) and siRNAs bind to complementary mRNA and recruit factors to degrade the target mRNA, modulating the target mRNA's protein expression to yield a pharmacological response (Nucleic Acids Research, 2018, 46, 1584-1600). Second-generation ASOs are typically 20 nucleotide-long phosphorothioate oligonucleotides containing a 10-nucleotide DNA “gap” and end-modified with 2′-O-methyl, 2′-O-methoxyethyl (MOE) or locked nucleic acid (LNA) nucleotides (Drug Discovery Today, 2018, 23, 101-114). There are several second-generation ASOs advanced to the clinic for a variety of indications, many of which target mRNA expressed primarily in the hepatocytes in the liver. Recently, conjugation of ASOs and siRNAs to tri-antennary GalNAc ligands has been shown to improve potency in hepatocytes (Molecular Therapy, 2019, 27, 1547-1555). GalNAc conjugation on both the 3′- and 5′-termini of oligonucleotides has been evaluated and both have significantly enhanced potency in cells and in animals (Bioconjugate Chem. 2015, 26, 1451-1455).

### DESCRIPTION OF THE RELATED ART

WO2009/002944A1 describes an iRNA agent that is conjugated with at least one (preferred di-antennary or tri-antennary) carbohydrate ligand. The carbohydrate-conjugated iRNA agents target, in particular, the parenchymal cells of the liver.

WO2015/042447A1 describes a series of branching groups which are conjugated therapeutic nucleoside agents and GalNAc ligands.

WO2017084987A1 describes the GalNAc phosphoramidite derivatives that can directly be introduced as building blocks together with nucleoside building blocks in solid phase oligonucleotide synthesis.

However, the synthesis of proper multivalent GalNAc ligands is not a trivial task, and it generally requires over 10 steps of chemical reactions. Here, we are providing improved GalNAc ligands by creating novel structures via introduction of long carbon chains for more efficient syntheses and longer durability of the GalNAc conjugates.

### BRIEF DESCRIPTION OF THE SEVERAL VIEWS OF THE DRAWINGS

FIG. 1A. The plasma ApoB levels of mice treated with oligonucleotides conjugated with the GalNAc clusters B001 without a spacer and positive control GalNAc cluster B005 through a spacer between GalNAc and oligonucleotide.

FIG. 1B. The plasma ApoB levels of mice treated with oligonucleotides conjugated with the GalNAc clusters B003 of the present disclosure and positive control GalNAc cluster B005 through a spacer between GalNAc and oligonucleotide.

FIG. 1C. The structure of the GalNAc cluster B003 of the present disclosure.

FIG. 2A. The ApoB levels in comparison between GalNAc-ApoB antisense conjugates B006 (Gr 3/4) of the present disclosure and positive control GalNAc cluster B005 (Gr 1/2) at both dose levels.

FIG. 2B. The ApoB levels in comparison between GalNAc-ApoB antisense conjugates B007 (Gr 5/6) of the present disclosure and positive control GalNAc cluster B005 (Gr 1/2) at both dose levels.

FIG. 2C. The ApoB levels in comparison between GalNAc-ApoB antisense conjugates B008 (Gr 7/8) of the present disclosure and positive control GalNAc cluster B005 (Gr 1/2) at both dose levels.

FIG. 2D. The ApoB levels in comparison between GalNAc-ApoB antisense conjugates B009 (Gr 9/10) of the present disclosure and positive control GalNAc cluster B005 (Gr 1/2) at both dose levels.

FIG. 2E. The ApoB levels in comparison between GalNAc-ApoB antisense conjugates B011 (Gr 11/12) of the present disclosure and positive control GalNAc cluster B005 (Gr 1/2) at both dose levels.

FIG. 2F. The ApoB levels in comparison between GalNAc-ApoB antisense conjugates B013 (Gr 13/14) of the present disclosure and positive control GalNAc cluster B005 (Gr 1/2) at both dose levels.

FIG. 2G. The ApoB levels in comparison between GalNAc-ApoB antisense conjugates B015 (Gr 15/16) of the present disclosure and positive control GalNAc cluster B005 (Gr 1/2) at both dose levels.

FIG. 3. The standard synthetic cycles for oligonucleotide syntheses used on DNA/RNA synthesizer on universal linker solid support.

### BRIEF SUMMARY

The present disclosure relates to a series of conjugates, conjugated antisense oligonucleotide agents (which may be used as therapeutic agents), methods of preparing the conjugates and conjugated antisense oligonucleotide agents, and methods of reducing the amount or activity of a nucleic acid transcript in a cell comprising contacting a cell with a conjugated antisense agent.

In certain embodiments, the present disclosure relates to conjugates having the structure of Formula (I):

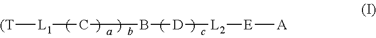

wherein:
T is a cell-targeting ligand;
L₁ and L₂ are independently a tether group;
C is a linker group;
B is a branching group;
D is linker group
E is ester group;
A is an antisense sequence or passenger strand of siRNA;
a is 0 or 1;
b is an integer between 1-5; and
c is 1 or 2.

In certain embodiments, the present disclosure relates to conjugated antisense oligonucleotide agents comprising the conjugates of Formula (I) and an oligonucleotide.

In certain embodiments, the present disclosure also relates to conjugates having di-antennary, tri-antennary, tetra-antennary, penta-antennary, or hexa-antennary cell-targeting ligands.

In certain embodiments, the present disclosure also relates to a conjugated antisense oligonucleotide agent (which may be used as a therapeutic agent), RNA agent, or DNA agent comprising a conjugate and an antisense or siRNA oligonucleotide.

In certain embodiments, the present disclosure also relates to methods of preparing the conjugates and their conjugation to oligonucleotides.

The new conjugates can be easily synthesized, and they easily facilitate the engagement of cell-targeting ligands to increase the delivery of, e.g., antisense or siRNA oligonucleotides, or open new pathways to conjugate multiple ASOs on a single molecule to increase delivery effectiveness.

### DETAILED DESCRIPTION

### Conjugates Structure

Some embodiments of the conjugates of the present disclosure include a compound of formula (I):

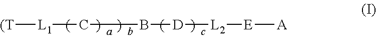

wherein:
T is a cell-targeting ligand;
L₁ and L₂ are independently a tether group;
C is a linker group;
B is a branching group;
D is linker group
E is ester group;
A is an antisense sequence or passenger strand of siRNA;
a is 0 or 1;
b is an integer between 1-5; and
c is 1 or 2.

In some embodiments, T is selected to have an affinity for at least one type of receptor on a target cell. In some embodiments, T is selected to have an affinity for at least one type of receptor on the surface of a mammalian liver cell. In some embodiments, T is a carbohydrate, carbohydrate derivative, modified carbohydrate, multivalent carbohydrate cluster, polysaccharide, modified polysaccharide, or polysaccharide derivative. In some embodiments, each T is independently selected from a carbohydrate, an amino sugar or a thio sugar. For example, in some embodiments, T is a carbohydrate selected from glucose, mannose, galactose, or fucose. For example, in some embodiments, T is an amino sugar selected from any number of compounds known in the art, for example glucosamine, sialic acid, α-D-galactosamine, N-Acetylgalactosamine, 2-acetamido-2-deoxy-D-galactopyranose (GalNAc), 2-Amino-3-O—[(R)-1-carboxyethyl]-2-deoxy-β-D-glucopyranose (β-muramic acid), 2-Deoxy-2-methylamino-L-glucopyranose, 4,6-Dideoxy-4-formamido-2,3-di-O-methyl-D-mannopyranose, 2-Deoxy-2-sulfoamino-D-glucopyranose, N-sulfo-D-glucosamine, or N-Glycoloyl-α-neuraminic acid. For example, thio sugars may be selected from the group consisting of 5-Thio-β-D-glucopyranose, Methyl 2,3,4-tri-O-acetyl-1-thio-6-O-trityl-α-D-glucopyranoside, 4-Thio-β-D-galactopyranose, or ethyl 3,4,6,7-tetra-O-acetyl-2-deoxy-1,5-dithio-α-D-gluco-heptopyranoside. Preferably, T is 2-acetamido-2-deoxy-D-galactopyranose (GalNAc).

In some embodiments, L₁ and L₂ are selected from C₁-C₂₀ alkylene, amide, or (C₁-C₂₀) alkylene-amide-(C₁-C₂₀) alkylene. In some embodiments, L₁ and L₂ are selected from C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkylene, amide, C₁, C₂, C₃, C₄, C₅, C₆, C₇, C₈, C₉, or C₁₀ alkylene-amide-C₁, C₂, C₃, C₄, C₅, C₆, C₇, C₈, C₉, or C₁₀ alkylene.

In some embodiments, L₁ and L₂ are independently selected from —(CH₂)ₙ—, —(CH₂)ₘ—CONH—(CH₂)ₘ—, or —(CH₂)ₘ—NHCO—(CH₂)ₘ—, m is an integer between 1-10; and n is an integer between 5-20. In some embodiments, m is selected from 1, 2, 3, 4, 5, 6, 7, 8, 9, or 10; and n is selected from 1, 2, 3, 4, 5, 6, 7, 8, 9, or 10.

In some embodiments, C is selected from C₁-C₂₀ alkylene, amide, carbonyl, amide-(C₁-C₂₀) alkylene, or carbonyl-heterocyclic ring-phosphate-(C₁-C₁₀) alkylene. In some embodiments, C is selected from C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkylene, amide, or amide-(C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀) alkylene. In some embodiments, C is selected from carbonyl-heterocyclic ring-phosphate-(C₁, C₂, C₃, C₄, C₅, C₆, C₇, C₈, C₉, or C₁₀) alkylene, wherein a heterocyclic ring means a 5- to 7-membered monocyclic, or 7- to 10-membered bicyclic, heterocyclic ring which is either saturated, unsaturated, or aromatic, and which contains from 1 or 2 heteroatoms independently selected from nitrogen, oxygen and sulfur, and wherein the nitrogen and sulfur heteroatoms may be optionally oxidized, and the nitrogen heteroatom may be optionally quaternized, including bicyclic rings in which any of the above heterocycles are fused to a benzene ring. The heterocycle may be attached via any heteroatom or carbon atom. Heterocycles include heteroaryls as defined below. Heterocycles include morpholinyl, pyrrolidinonyl, pyrrolidinyl, piperidinyl, piperizynyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

In some embodiments, C is selected from:

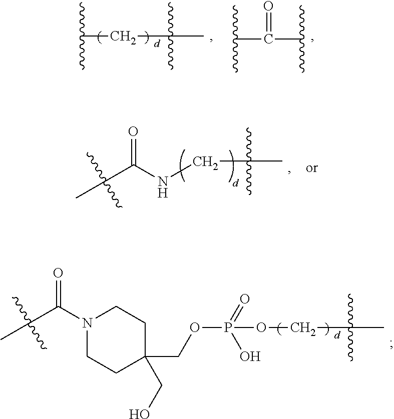

wherein d is an integer between 0-5.

In some embodiments, B is di-antennary branching group, tri-antennary branching group, tetra-antennary branching group, penta-antennary branching group, or hexa-antennary branching group.

In some embodiments, B is selected from:

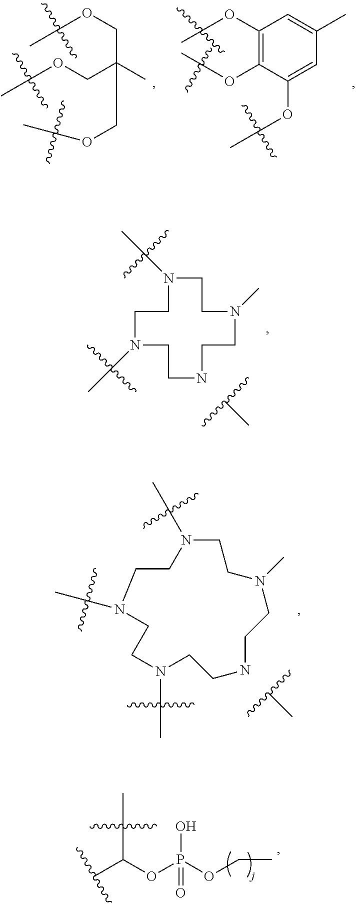

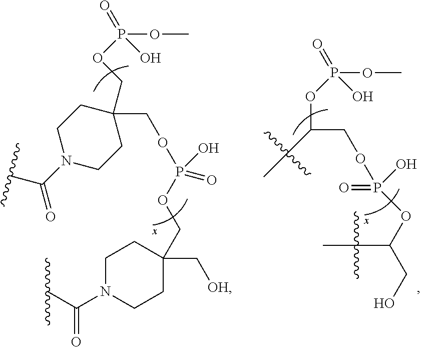

wherein x is an integer between 1-5; and

j is an integer between 0-5.

In some embodiments, D is selected from a straight or branched C₁-C₂₀ alkylene, amide, carbonyl, or (C₁-C₂₀) alkylene-amide-(C₁-C₂₀) alkylene. In some embodiments, D is selected from C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkylene, amide, carbonyl, or (C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀) alkylene-amide-(C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀) alkylene. In some embodiments, D is selected from —(CH₂)ₖ—,

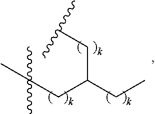

—(C═O)—, —CONH—, or —NHCO—; wherein k is an integer between 0-5.

In some embodiments, E is phosphate, thiophosphate, dithiophosphate, or boranophosphate.

In some embodiments, E is

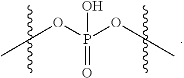 [SMILES: *OP(=O)(O)O*]

In some embodiments, conjugates are provided having the following structure:

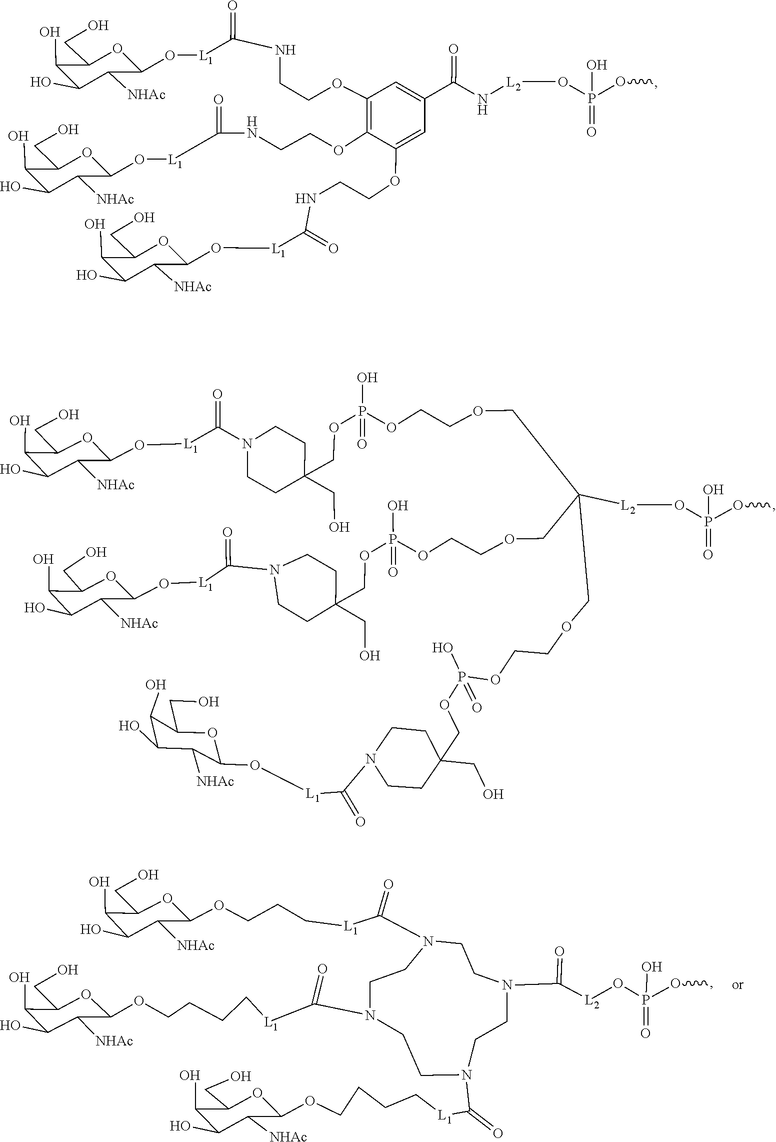

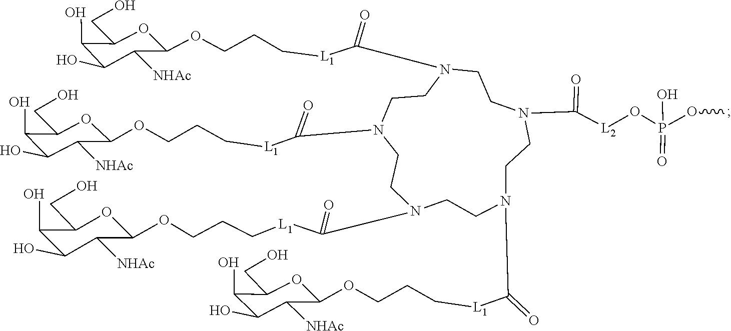

wherein L₁ and L₂ have the same definition as above.

In some embodiments, conjugates are provided having the following structure:

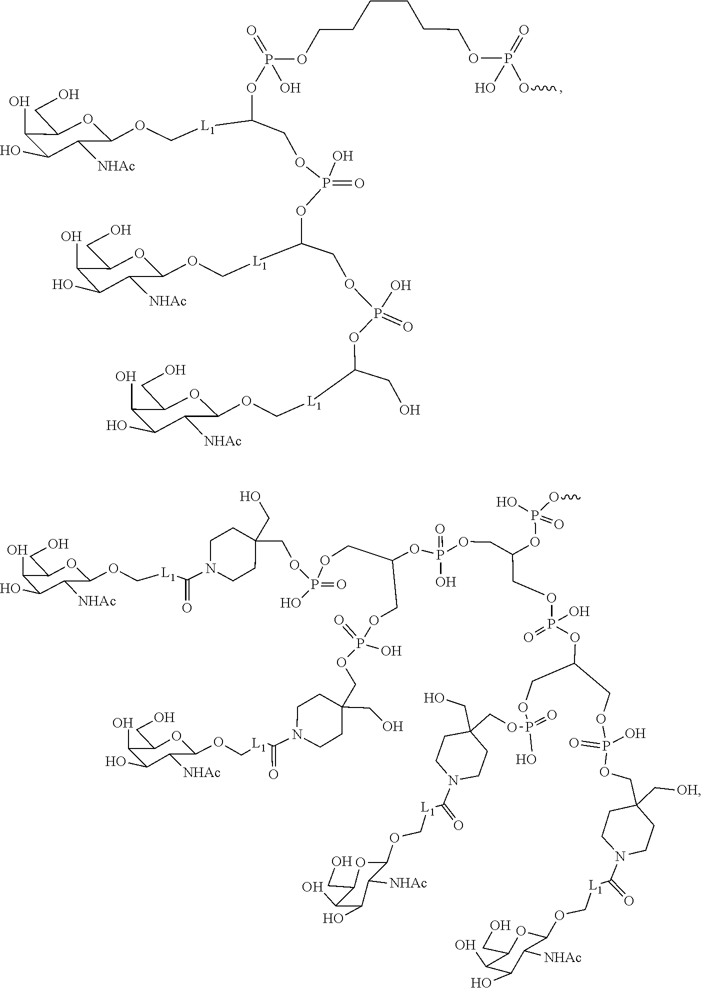

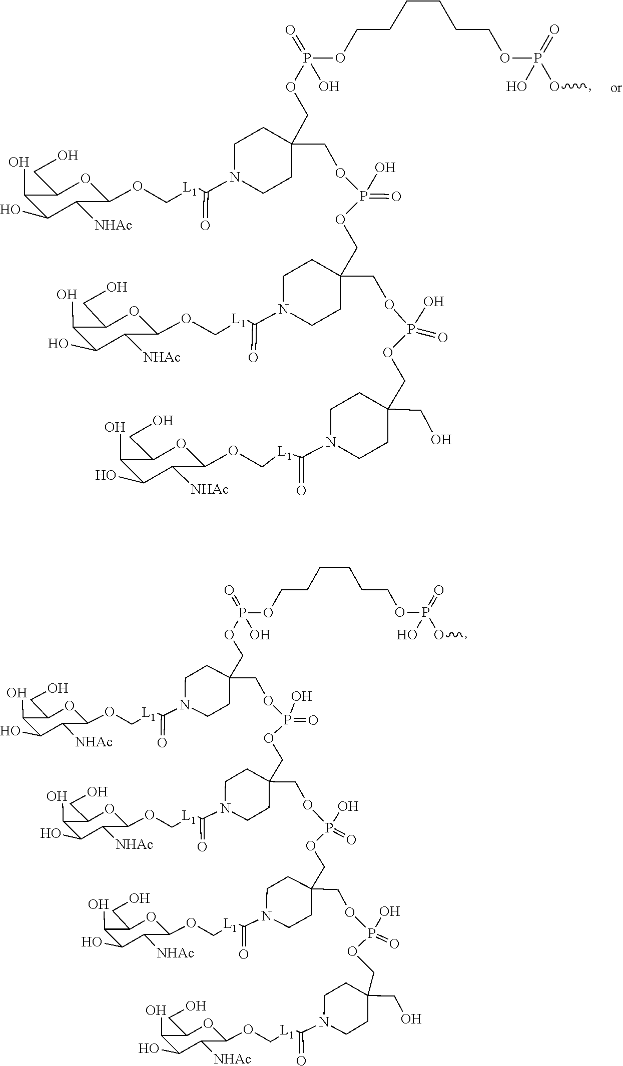

wherein L₁ has the same definition as above.

### Oligonucleotide Agent

The present disclosure relates to a series of oligonucleotide (RNA/DNA) agents, which comprises conjugate and antisense oligonucleotides.

Exemplary oligonucleotide agents comprising the conjugate structures of the present disclosure include those listed in the examples.

In some embodiments, the antisense oligonucleotides are linked to the conjugates through the “E” group (e.g. phosphate).

In some embodiments, the conjugates enhance the activity, cellular distribution, or cellular uptake of the oligonucleotide by a particular type of cell, such as hepatocytes.

In some embodiments, the oligonucleotide sequences described herein are conjugated or modified at one or both ends by each conjugate moiety of the present disclosure. In some embodiments, the oligonucleotide strand comprises a conjugate moiety of the present disclosure conjugated at the 5′ and/or 3′ end through the “E” group (e.g. phosphate). In some embodiments, the conjugate moiety of the present disclosure is conjugated at the 3′-end of the oligonucleotide strand. In some embodiments, the conjugate moiety of the present disclosure is conjugated on the nucleosides in the middle of the oligonucleotide strand.

In certain embodiments, the conjugated antisense oligonucleotide agents (which may be used as therapeutic agents) comprise an HBV antisense oligonucleotide (HBV ASO) known in the art and a conjugate group. Examples of HBV ASO for conjugation include but are not limited to those disclosed in Table 1.

TABLE 1
Example of base sequences targeted to HBV

| Seq ID | |
|---|---|
| No. | Sequence of HBV ASO (5’-3’) |
| 1 | GAGAGAAGTCCACCAC |
| 2 | TGAGAGAAGTCCACCA |
| 3 | GAGGCATAGCAGCAGG |
| 4 | TGAGGCATAGCAGCAG |
| 5 | GATGAGGCATAGCAGC |
| 6 | GATGGGATGGGAATAC |
| 7 | GGCCCACTCCCATAGG |
| 8 | AGGCCCACTCCCATAG |
| 9 | CTGAGGCCCACTCCCA |
| 10 | GCAGAGGTGAAGCGAAGTGC |
| 11 | CCACGAGTCTAGACTCT |
| 12 | GTCCACCACGAGTCTAG |
| 13 | AGTCCACCACGAGTCTA |
| 14 | ANGTCCACCACGAGTCT |
| 15 | GAAGTCCACCACGAGTC |
| 16 | AGAAGTCCACCACGAGT |
| 17 | GAGAAGTCCACCACGAG |
| 18 | AGAGAAGTCCACCACGA |
| 19 | GAGAGAAGTCCACCACG |
| 20 | TGAGAGAAGTCCACCAC |
| 21 | TGATAAAACGCCGCAGA |
| 22 | ATGATAAAACGCCGCAG |
| 23 | GGCATAGCAGCAGGATG |
| 24 | AGGCATAGCAGCAGGAT |
| 25 | GAGGCATAGCAGCAGGA |
| 26 | AGATGAGGCATAGCAGCAGG |
| 27 | AAGATGAGGCATAGCAGCAG |
| 28 | ATGAGGCATAGCAGCAG |
| 29 | GAAGATGAGGCATAGCAGCA |
| 30 | GATGAGGCATAGCAGCA |
| 31 | AGAAGATGAGGCATAGCAGC |
| 32 | AGATGAGGCATAGCAGC |
| 33 | AAGAAGATGAGGCATAGCAG |
| 34 | AAGATGAGGCATAGCAG |
| 35 | AGAAGATGAGGCATAGC |
| 36 | AAGAAGATGAGGCATAG |
| 37 | ACGGGCAACATACCTTG |
| 38 | CTGAGGCCCACTCCCATAGG |
| 39 | AGGCCCACTCCCATAGG |
| 40 | GAGGCCCACTCCCATAG |
| 41 | TGAGGCCCACTCCCATA |
| 42 | CTGAGGCCCACTCCCAT |
| 43 | CGAACCACTGAACAAATGGC |
| 44 | ACCACTGAACAAATGGC |
| 45 | AACCACTGAACAAATGG |
| 46 | GAACCACTGAACAAATG |
| 47 | CGAACCACTGAACAAAT |
| 48 | ACCACATCATCCATATA |
| 49 | TCAGCAAACACTTGGCA |
| 50 | AATTTATGCCTACAGCCICC |
| 51 | TTATGCCTACAGCCTCC |
| 52 | CAATTTATGCCTACAGCCTC |
| 53 | TTTATGCCTACAGCCTC |
| 54 | CCAATTTATGCCTACAGCCT |
| 55 | ATTTATGCCTACAGCCT |
| 56 | ACCAATTTATGCCTACAGCC |
| 57 | AATTTATGCCTACAGCC |
| 58 | CAATTTATGCCTACAGC |
| 59 | CCAATTTATGCCTACAG |
| 60 | ACCAATTTATGCCTACA |
| 61 | AGGCAGAGGTGAAAAAG |
| 62 | TAGGCAGAGGTGAAAAA |
| 63 | GCACAGCTTGGAGGCTTGAA |
| 64 | CAGCTTGGAGGCTTGAA |
| 65 | GGCACAGCTTGGAGGCTTGA |
| 66 | ACAGCTTGGAGGCTTGA |
| 67 | AGGCACAGCTTGGAGGCTTG |
| 68 | CACAGCTTGGAGGCTTG |
| 69 | AAGGCACAGCTTGGAGGCTT |
| 70 | GCACAGCTTGGAGGCTT |
| 71 | CAAGGCACAGCTTGGAGGCT |
| 72 | GGCACAGCTTGGAGGCT |
| 73 | CCAAGGCACAGCTTGGAGGC |
| 74 | AGGCACAGCTTGGAGGC |
| 75 | AAGGCACAGCTTGGAGG |
| 76 | CAAGGCACAGCTTGGAG |
| 77 | CCAAGGCACAGCTTGGA |
| 78 | GCTCCAAATTCTTTATA |
| 79 | TCTGCGAGGCGAGGGAGTTC |
| 80 | GCGAGGCGAGGGAGTTC |
| 81 | TGCGAGGCGAGGGAGTT |
| 82 | CTGCGAGGCGAGGGAGT |
| 83 | TCTGCGAGGCGAGGGAG |
| 84 | TTCCCAAGAATATGGTG |
| 85 | GTTCCCAAGAATATGGT |
| 86 | TGTTCCCAAGAATATGG |

TABLE 2
HBV sense and antisense sequence

| Seq ID | | Seq ID | |
|---|---|---|---|
| No. | HBV Sense | No. | HBV Antisense |
| 87 | UCGUGGUGGACUUCUCUCA | 88 | UGAGAGAAGUCCACCACGA |
| 89 | GUGGUGGACUUCUCUCAAU | 90 | AUUGAGAGAAGUCCACCAC |
| 91 | GCCGAUCCAUACUGCGGAA | 92 | UUCCGCAGUAUGGAUCGGC |
| 93 | CCGAUCCAUACUGCGGAAC | 94 | GUUCCGCAGUAUGGAUCGG |
| 95 | CAUCCUGCUGCUAUGCCUC | 96 | GAGGCAUAGCAGCAGGAUG |
| 97 | UGCUGCUAUGCCUCAUCUU | 98 | AAGAUGAGGCAUAGCAGCA |
| 99 | GGUGGACUUCUCUCAAUUU | 100 | AAAUUGAGAGAAGUCCACC |
| 101 | UGGUGGACUUCUCUCAAUU | 102 | AAUUGAGAGAAGUCCACCA |
| 103 | UAGACUCGUGGUGGACUUC | 104 | GAAGUCCACCACGAGUCUA |
| 105 | UCCUCUGCCGAUCCAUACU | 106 | AGUAUGGAUCGGCAGAGGA |
| 107 | UGCCGAUCCAUACUGCGGA | 108 | UCCGCAGUAUGGAUCGGCA |
| 109 | UGGAUGUGUCUGCGGCGUU | 110 | AACGCCGCAGACACAUCCA |
| 111 | CGAUCCAUACUGCGGAACU | 112 | AGUUCCGCAGUAUGGAUCG |
| 113 | CGCACCUCUCUUUACGCGG | 114 | CCGCGUAAAGAGAGGUGCG |
| 115 | CUGCCGAUCCAUACUGCGG | 116 | CCGCAGUAUGGAUCGGCAG |
| 117 | CGUGGUGGACUUCUCUCAA | 118 | UUGAGAGAAGUCCACCACG |
| 119 | CUGCUGCUAUGCCUCAUCU | 120 | AGAUGAGGCAUAGCAGCAG |
| 121 | CCUGCUGCUAUGCCUCAUC | 122 | GAUGAGGCAUAGCAGCAGG |
| 123 | CUAGACUCGUGGUGGACUU | 124 | AAGUCCACCACGAGUCUAG |
| 125 | UCCUGCUGCUAUGCCUCAU | 126 | AUGAGGCAUAGCAGCAGGA |
| 127 | GACUCGUGGUGGACUUCUC | 128 | GAGAAGUCCACCACGAGUC |
| 129 | AUCCAUACUGCGGAACUCC | 130 | GGAGUUCCGCAGUAUGGAU |
| 131 | CUCUGCCGAUCCAUACUGC | 132 | GCAGUAUGGAUCGGCAGAG |
| 133 | GAUCCAUACUGCGGAACUC | 134 | GAGUUCCGCAGUAUGGAUC |
| 135 | GAAGAACUCCCUCGCCUCG | 136 | CGAGGCGAGGGAGUUCUUC |
| 137 | AAGCCUCCAAGCUGUGCCU | 138 | AGGCACAGCUUGGAGGCUU |
| 139 | AGAAGAACUCCCUCGCCUC | 140 | GAGGCGAGGGAGUUCUUCU |
| 141 | GGAGUGUGGAUUCGCACUC | 142 | GAGUGCGAAUCCACACUCC |
| 143 | CCUCUGCCGAUCCAUACUG | 144 | CAGUAUGGAUCGGCAGAGG |
| 145 | CAAGCCUCCAAGCUGUGCC | 146 | GGCACAGCUUGGAGGCUUG |
| 147 | UCCAUACUGCGGAACUCCU | 148 | AGGAGUUCCGCAGUAUGGA |
| 149 | CAGAGUCUAGACUCGUGGU | 150 | ACCACGAGUCUAGACUCUG |
| 151 | AAGAAGAACUCCCUCGCCU | 152 | AGGCGAGGGAGUUCUUCUU |
| 153 | GAGUGUGGAUUCGCACUCC | 154 | GGAGUGCGAAUCCACACUC |
| 155 | UCUAGACUCGUGGUGGACU | 156 | AGUCCACCACGAGUCUAGA |
| 157 | GCUGCUAUGCCUCAUCUUC | 158 | GAAGAUGAGGCAUAGCAGC |
| 159 | AGUCUAGACUCGUGGUGGA | 160 | UCCACCACGAGUCUAGACU |
| 161 | CUCCUCUGCCGAUCCAUAC | 162 | GUAUGGAUCGGCAGAGGAG |
| 163 | UGGCUCAGUUUACUAGUGC | 164 | GCACUAGUAAACUGAGCCA |
| 165 | GUCUAGACUCGUGGUGGAC | 166 | GUCCACCACGAGUCUAGAC |
| 167 | UUCAAGCCUCCAAGCUGUG | 168 | CACAGCUUGGAGGCUUGAA |
| 169 | CUAUGGGAGUGGGCCUCAG | 170 | CUGAGGCCCACUCCCAUAG |
| 171 | CUCGUGGUGGACUUCUCUC | 172 | GAGAGAAGUCCACCACGAG |
| 173 | CCUAUGGGAGUGGGCCUCA | 174 | UGAGGCCCACUCCCAUAGG |
| 175 | AAGAACUCCCUCGCCUCGC | 176 | GCGAGGCGAGGGAGUUCUU |
| 177 | UCUGCCGAUCCAUACUGCG | 178 | CGCAGUAUGGAUCGGCAGA |
| 179 | AGAGUCUAGACUCGUGGUG | 180 | CACCACGAGUCUAGACUCU |
| 181 | GAAGAAGAACUCCCUCGCC | 182 | GGCGAGGGAGUUCUUCUUC |
| 183 | UCAAGCCUCCAAGCUGUGC | 184 | GCACAGCUUGGAGGCUUGA |
| 185 | AGCCUCCAAGCUGUGCCUU | 186 | AAGGCACAGCUUGGAGGCU |
| 187 | AGACUCGUGGUGGACUUCU | 188 | AGAAGUCCACCACGAGUCU |
| 189 | GUGUGCACUUCGCUUCACA | 190 | UGUGAAGCGAAGUGCACACUU |
| 191 | CACCAUGCAACUUUUUCACCU | 192 | AGGUGAAAAAGUUGCAUGGUGUU |
| 193 | AUCCAUACUGCGGAACUCC | 194 | GGAGUUCCGCAGUAUGGAU |
| 195 | CUCUGCCGAUCCAUACUGC | 196 | GCAGUAUGGAUCGGCAGAG |
| 197 | GAUCCAUACUGCGGAACUC | 198 | GAGUUCCGCAGUAUGGAUC |
| 199 | GAAGAACUCCCUCGCCUCG | 200 | CGAGGCGAGGGAGUUCUUC |
| 201 | AAGCCUCCAAGCUGUGCCU | 202 | AGGCACAGCUUGGAGGCUU |
| 203 | AGAAGAACUCCCUCGCCUC | 204 | GAGGCGAGGGAGUUCUUCU |
| 205 | GGAGUGUGGAUUCGCACUC | 206 | GAGUGCGAAUCCACACUCC |
| 207 | CCUCUGCCGAUCCAUACUG | 208 | CAGUAUGGAUCGGCAGAGG |
| 209 | CAAGCCUCCAAGCUGUGCC | 210 | GGCACAGCUUGGAGGCUUG |
| 211 | UCCAUACUGCGGAACUCCU | 212 | AGGAGUUCCGCAGUAUGGA |
| 213 | CAGAGUCUAGACUCGUGGU | 214 | ACCACGAGUCUAGACUCUG |
| 215 | AAGAAGAACUCCCUCGCCU | 216 | AGGCGAGGGAGUUCUUCUU |
| 217 | GAGUGUGGAUUCGCACUCC | 218 | GGAGUGCGAAUCCACACUC |
| 219 | UCUAGACUCGUGGUGGACU | 220 | AGUCCACCACGAGUCUAGA |
| 221 | GCUGCUAUGCCUCAUCUUC | 222 | GAAGAUGAGGCAUAGCAGC |
| 223 | AGUCUAGACUCGUGGUGGA | 224 | UCCACCACGAGUCUAGACU |
| 225 | CUCCUCUGCCGAUCCAUAC | 226 | GUAUGGAUCGGCAGAGGAG |
| 227 | UGGCUCAGUUUACUAGUGC | 228 | GCACUAGUAAACUGAGCCA |
| 229 | GUCUAGACUCGUGGUGGAC | 230 | GUCCACCACGAGUCUAGAC |
| 231 | UUCAAGCCUCCAAGCUGUG | 232 | CACAGCUUGGAGGCUUGAA |
| 233 | CUAUGGGAGUGGGCCUCAG | 234 | CUGAGGCCCACUCCCAUAG |
| 235 | CUCGUGGUGGACUUCUCUC | 236 | GAGAGAAGUCCACCACGAG |
| 237 | CCUAUGGGAGUGGGCCUCA | 238 | UGAGGCCCACUCCCAUAGG |
| 239 | AAGAACUCCCUCGCCUCGC | 240 | GCGAGGCGAGGGAGUUCUU |
| 241 | UCUGCCGAUCCAUACUGCG | 242 | CGCAGUAUGGAUCGGCAGA |
| 243 | AGAGUCUAGACUCGUGGUG | 244 | CACCACGAGUCUAGACUCU |
| 245 | GAAGAAGAACUCCCUCGCC | 246 | GGCGAGGGAGUUCUUCUUC |
| 247 | CCGUGUGCACUUCGCUUCAUU | 248 | UGAAGCGAAGUGCACACGGUU |
| 249 | CUGGCUCAGUUUACUAGUGUU | 250 | CACUAGUAAACUGAGCCAGUU |
| 251 | GCCGAUCCAUACUGCGGAAUU | 252 | UUCCGCAGUAUGGAUCCGCUU |
| 253 | AGGUAUGUUGCCCGUUUGUUU | 254 | ACAAACGGGCAACAUACCUUU |
| 255 | GCUCAGUUUACUAGUGCCAUU | 256 | UGGCACUAGUAAACUGAGCUU |
| 257 | CAAGGUAUGUUGCCCGUUUUU | 258 | AAACGGGCAACAUACCUUGUU |
| 259 | CUGUAGGCAUAAAUUGGUAUU | 260 | UACCAAUUUAUGCCUACAGUU |
| 261 | UCUGCGGCGUUUUAUCAUAUU | 262 | UAUGAUAAAACGCCGCAGAUU |
| 263 | ACCUCUGCCUAAUCAUCUCUUU | 264 | GAGAUGAUUAGGCAGAGGUUU |
| 265 | UUUACUAGUGCCAUUUGUAUU | 266 | UACAAAUGGCACUAGUAAAUU |
| 267 | ACCUCUGCCUAAUCAUCUAUU | 268 | UAGAUGAUUAGGCAGAGGUUU |
| 269 | CUGUAGGCAUAAAUUGGUCUU | 270 | GACCAAUUUAUGCCUACAGUU |
| 271 | CCGUGUGCACUUCGCUUCAUU | 272 | UGAAGCGAAGUGCACACGGUU |

In certain embodiments, the conjugated antisense oligonucleotide agents (which may be used as therapeutic agents) comprise an antisense oligonucleotide having a nucleobase sequence of any of SEQ ID NOs 321/485; 322/486; 324/488; 325/489; 326/490; 327/491; 328/492 and 350/514 disclosed in WO/2013/003520 and a conjugate group described herein. In certain embodiments, the conjugated antisense oligonucleotide agents (which may be used as therapeutic agents) comprise an antisense oligonucleotide having a nucleobase sequence of any of SEQ ID NOs 3/5; 21/22 or HBV-219 disclosed in WO/2019/079781 and a conjugate group described herein. In certain embodiments, the conjugated antisense oligonucleotide agents (which may be used as therapeutic agents) comprise an antisense oligonucleotide having a nucleobase sequence of any of SEQ ID NOs 867-941 disclosed in WO 2017/015175 and a conjugate group described herein. In certain embodiments, the conjugated antisense oligonucleotide agents (which may be used as therapeutic agents) comprise an antisense oligonucleotide having a nucleobase sequence of (AC)ₙ (wherein n=15-20) disclosed in WO2020/097342 and a conjugate group described herein. The siRNA or antisense oligonucleotide sequences of all of the aforementioned referenced SEQ ID NOs. are incorporated by reference herein.

### Methods of Use

One aspect of the present technology includes methods for treating a subject diagnosed as having, suspected to have, or at risk of having any diseases that could be relieved by targeting the liver. One example is an HBV infection and/or an HBV-associated disorder. In therapeutic applications, compositions comprising the targeting group (e.g. GalNAc) conjugated oligonucleotides of the present technology are administered to a subject suspected of or already suffering from such a disease (such as, e.g., presence of an HBV surface antigen and envelope antigens (e.g., HBsAg and/or HBeAg) in the serum and/or liver of the subject, or elevated HBV DNA or HBV viral load levels), in an amount sufficient to cure, or at least partially arrest, the symptoms of the disease, including its complications and intermediate pathological phenotypes in development of the disease.

In some embodiments, the oligonucleotide agents of the present technology are used in the treatment of a metabolic disease or disorder, such as a hepatic disease or disorder; or are used in the treatment of hepatitis, such as hepatitis B or C.

Other examples include but are not limited to Hereditary ATTR amyloidosis, acute hepatic porphyria, primary hyperoxaluria, hypercholesterolemia (PCSK9, Apo B), cardiovascular diseases (Lpa, ANGPTL3, ApoCIII), ATTR amyloidosis, complement-mediated disease (C3 and CFB), clotting disorder (Factor XI), NASH (PNPLA3 and DGAT2), alpha-1 antitrypsin deficiency disease, and ornithine transcarbamylase deficiency.

### EXAMPLES

### Method of Synthesis

### Example 1

### Synthesis of GalNAc Building Blocks (for GalNAc Phosphoramidite)

GalNAc building blocks were designed and synthesized with each one of the following reactive moieties for extension: (a) carboxylic acid such as G001; G002 and G003, (b) amine such as G004, G005, G006 and G012, (c) alcohol G007, (d). aldehyde G008, (e) alkene G009, (f) alkyne G010, and (g) azide G011 (Table 3). These reactive moieties can react with proper counterparts to form 1,2-diol and 1,3-diol intermediates.

TABLE 3
GalNAc building blocks with various reactive terminals.

| | |
|---|---|
| 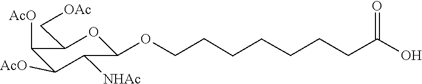 [SMILES: CC(=O)NC1C(OCCCCCCCC(=O)O)OC(COC(C)=O)C(OC(C)=O)C1OC(C)=O] | G001 |
| 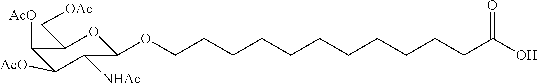 [SMILES: CC(=O)NC1C(OCCCCCCCCCCCC(=O)O)OC(COC(C)=O)C(OC(C)=O)C1OC(C)=O] | G002 |
| 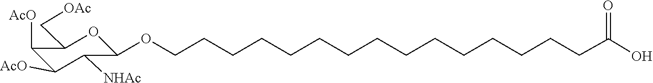 [SMILES: CC(=O)NC1C(OCCCCCCCCCCCCCCCC(=O)O)OC(COC(C)=O)C(OC(C)=O)C1OC(C)=O] | G003 |
| 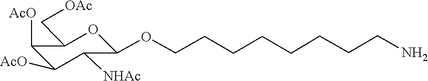 [SMILES: CC(=O)NC1C(OCCCCCCCCN)OC(COC(C)=O)C(OC(C)=O)C1OC(C)=O] | G004 |
| 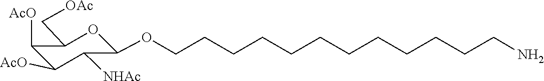 [SMILES: CC(=O)NC1C(OCCCCCCCCCCCCN)OC(COC(C)=O)C(OC(C)=O)C1OC(C)=O] | G005 |
| 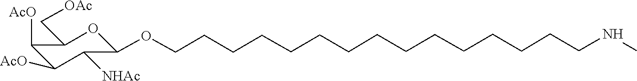 [SMILES: CNCCCCCCCCCCCCCCCOC1OC(COC(C)=O)C(OC(C)=O)C(OC(C)=O)C1NC(C)=O] | G006 |
| 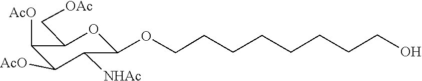 [SMILES: CC(=O)NC1C(OCCCCCCCCO)OC(COC(C)=O)C(OC(C)=O)C1OC(C)=O] | G007 |
| 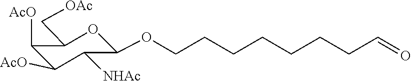 [SMILES: CC(=O)NC1C(OCCCCCCCC=O)OC(COC(C)=O)C(OC(C)=O)C1OC(C)=O] | G008 |
| 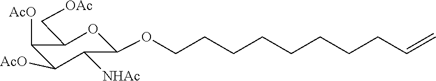 [SMILES: C=CCCCCCCCCOC1OC(COC(C)=O)C(OC(C)=O)C(OC(C)=O)C1NC(C)=O] | G009 |
| 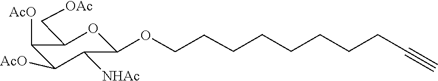 [SMILES: C#CCCCCCCCCOC1OC(COC(C)=O)C(OC(C)=O)C(OC(C)=O)C1NC(C)=O] | G010 |
| 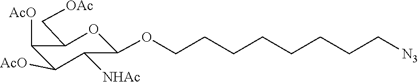 [SMILES: CC(=O)NC1C(OCCCCCCCCN=[N+]=[N-])OC(COC(C)=O)C(OC(C)=O)C1OC(C)=O] | G011 |
| 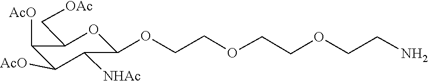 [SMILES: CC(=O)NC1C(OCCOCCOCCN)OC(COC(C)=O)C(OC(C)=O)C1OC(C)=O] | G012 |

A representative method and synthetic protocol are given below:

### Example 1-1

### Syntheses of G001

### Step 1 Synthesis of B

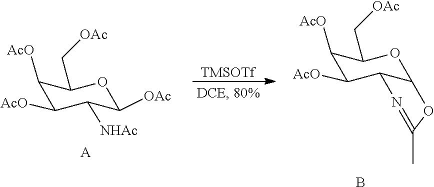

TMSOTf (10.85 mL, 60.0 mmol) was added to aminosugar pentaacetate A (15.5 g, 39.85 mmol) in dichloroethane (90 mL) dropwise. The mixture was heated to 50° C. for 1.5 hours and stirred at ambient temperature overnight. The reaction was quenched by cold aq. sat. NaHCO₃ and extracted with DCM (3×300 mL). The combined organic layers were washed with H₂O, dried over Na₂SO₄, filtered, and evaporated in vacuo to give a residue of B, 10.5 g (˜80%) without further purification.

### Step 2 Synthesis of C

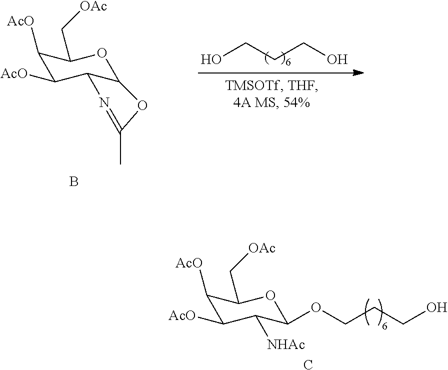

B (4.28 g, 13.0 mmol) was dissolved in anhydrous THF (40 mL) and stirred with 4 Å molecular sieves at ambient temperature for 5 minutes before the addition of 1,8-diol (2.09 g, 14.3 mmol). The mixture was stirred for 30 minutes and TMSOTf (1.18 mL, 6.5 mmol) was added dropwise. The resulting mixture was stirred overnight, and the reaction was quenched by cold aq. sat. NaHCO₃ and extracted with DCM (3×100 mL). The combined organic layers were washed with H₂O, dried over Na₂SO₄, filtered, and evaporated in vacuo to give a residue. The residue was purified on a silica gel column to yield 4.01 g (65%) of C.

### Step 3 Synthesis of D

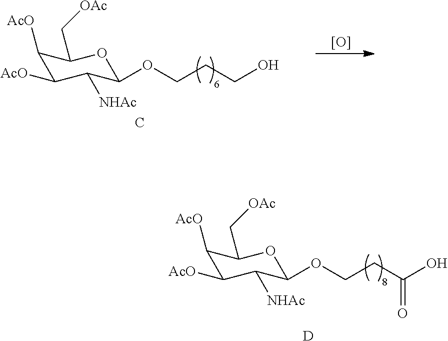

C (4 g, 8.42 mmol) in a 500 mL round-bottom flask was added TEMPO (0.75 g, 4.8 mmol), 43 mL of acetonitrile, and 120 mL of 0.67 M sodium phosphate buffer with agitation and the resulting mixture was heated to 35° C. A solution of sodium chlorite (32.5 mL, prepared by dissolving 9.14 g of NaClO₂ in 40 mL H₂O) and a solution of sodium hypochlorite (16.25 mL, prepared by diluting household bleach (5.25% NaOCl, 1.06 mL, ca. 2.0 mol %) with 19 mL of H₂O were added to the reaction mixture over 2 hrs in 5 batches. The reaction was stirred at 35° C. for 16 hrs, quenched with Na₂S₂O₃, and acidified with saturated NH₄Cl. The mixture was extracted with ethyl acetate (3×100 mL) and the combined organic layers were washed with H₂O, dried over MgSO₄, filtered, and evaporated in vacuo to give a residue. The residue was purified by silica gel column to yield 3.75 g (91%) of D.

[M+H]⁺=489.6. ¹H NMR (400 MHz, DMSO-d₆) δ 11.96 (s, 1H), 7.80 (d, J=9.2 Hz, 1H), 5.21 (d, J=3.4 Hz, 1H), 4.96 (dd, J=11.2, 3.5 Hz, 1H), 4.48 (d, J=8.5 Hz, 1H), 4.02 (m, 3H), 3.86 (dt, J=11.2, 8.8 Hz, 1H), 3.69 (dt, J=9.9, 6.2 Hz, 1H), 3.41 (dt, J=9.9, 6.5 Hz, 1H), 2.18 (t, J=7.4 Hz, 2H), 2.10 (s, 3H), 1.99 (s, 3H), 1.89 (s, 3H), 1.76 (s, 3H), 1.47 (m, 5H), 1.24 (s, 7H) ppm.

### Example 1-2

### Synthesis of G004

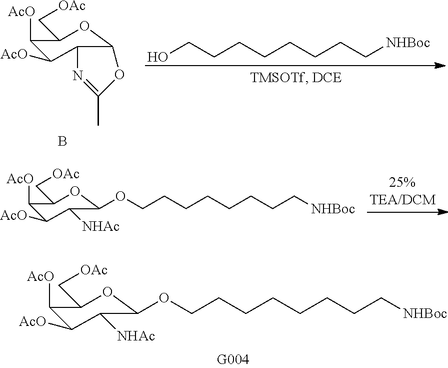

To a solution of B (10 g, 30.6 mmol) and tert-butyl (8-hydroxyoctyl)carbamate (9 g, 36.7 mmol) in 300 mL of 1,2-dichloroethane under an inert atmosphere of nitrogen was dropwise added TMSOTf (2.7 mL, 15.3 mmol) at 0° C. The resulting solution was stirred at room temperature for 16 h. The reaction mixture was quenched by the addition of ice/water (100 mL) and then extracted with dichloromethane (200 mL×2). The combined organic phases were washed with water (100 mL), and then dried over anhydrous sodium sulfate. The filtrate was concentrated under reduced pressure. The residue was purified with silica gel column eluted by PE/EA (1/2) first, and then purified by flash chromatography on reverse phase silica gel (ACN/H₂O=5%-95%, 214 nm, 30 min) to give Boc-protected G004 (4 g, 23.5% yield) as a white solid. MS Calcd: 574.3; Found: 575.3 [M+H]⁺. ¹H NMR (400 MHz, DMSO-d₆): δ 7.81 (d, J=9.2 Hz, 1H), 6.76-6.74 (m, 1H) , 5.21 (d, J=3.2 Hz, 1H), 4.98-4.95 (m, 1H), 4.48 (d, J=8.8 Hz, 1H), 4.04-4.00 (m, 3H), 3.90-3.83 (m, 1H), 3.72-3.66 (m, 1H), 3.43-3.32 (m, 1H), 2.90-2.85 (m, 2H), 2.10 (s, 3H), 2.00 (s, 3H), 1.89 (s, 3H), 1.77 (s, 3H), 1.45-1.44 (m, 2H), 1.37 (s, 11H), 1.23 (s, 8H).

G004 was generated by treating Boc-protected G004 in 25% trifluoracetic acid in dichloromethane at room temperature for 4h and removal of volatile material without further purification.

### Example 1-3

### Synthesis of G007

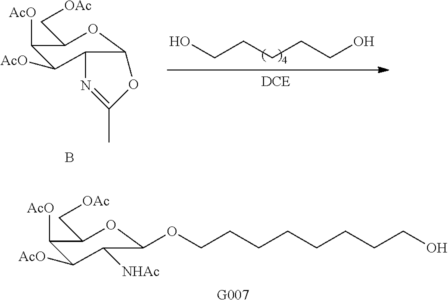

To a solution of compound B (10 g, 30.37 mmol) and octane-1,8-diol (4.44 g, 30.37 mmol) in 100 mL of DCE was added TMSOTf (3.38 g, 15.19 mmol) dropwise with stirring at 0° C. The resulting solution was stirred at room temperature for 16 h. The reaction was quenched with water (100 mL) and extracted with DCM (100 mL×3). The organic layer was concentrated, dried over anhydrous sodium sulfate, filtered, and concentrated under reduced pressure. The residue was purified by flash chromatography on reverse phase silica gel (ACN/H₂O=5%-95%, 214 nm, 30 min) to afford compound G007 (5.3 g, 37% yield) as a yellow solid. MS Calcd.: 475; MS Found: 476[M+H]⁺. ¹H NMR (400 MHz, DMSO-d₆) δ: 7.82 (d, J=9.2 Hz, 1H), 5.21 (d, J=3.6 Hz, 1H), 4.98-4.94 (m, 1H), 4.98 (d, J=8.4 Hz, 1H), 4.32 (s, 1H), 4.05-4.01 (m, 1H), 3.90-3.83 (m, 1H), 3.72-3.67 (m, 3H), 2.10 (s, 3H), 2.00 (s, 3H), 1.89 (s, 3H), 1.77 (s 3H), 1.45-1.38 (m, 4H), 1.24 (br, 8H).

### Example 1-4

### Synthesis of G010

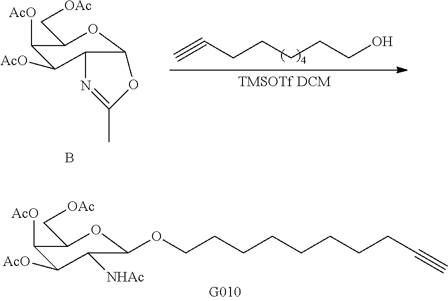

To a solution of compound B (5 g, 15.19 mmol) and decat-9-yn-1-ol (3.41 g, 30.37 mmol) in 100 mL of DCM was added TMSOTf (3.38 g, 15.19 mmol) dropwise with stirring at 0° C. The resulting solution was stirred at room temperature for 16 h. The reaction was quenched with H₂O (100 mL) and extracted with DCM (100 mL×3). The organic layer was concentrated. The organic layer was dried over anhydrous sodium sulfate, filtered, and concentrated under reduced pressure. The residue was purified by flash chromatography on reverse phase silica gel (ACN/H₂O=5%-95%, 214 nm, 30 min) to afford compound G010 (3.8 g, 83% yield) as a yellow solid. MS Calcd.: 483; MS Found: 484 [M+H]⁺. ¹H NMR (400 MHz, DMSO-d₆) δ: 7.80 (d, J=9.2 Hz, 1H), 5.21 (d, J=4.0 Hz, 1H), 4.97 (d, J=7.6 Hz, 1H), 4.48 (d, J=8.4 Hz, 1H), 4.04-4.01 (m, 3H), 3.90-3.83 (m, 1H), 3.72-3.67 (m, 1H), 3.44-3.38 (m, 1H), 2.71 (t, J=2.8 Hz, 1H), 2.16-2.10 (m, 2H), 2.00 (s, 3H), 1.89 (s, 3H), 1.77 (s, 3H), 1.46-1.41 (m, 4H), 1.35-1.32 (m, 2H), 1.25 (br, 6H)).

### Example 2

### A method of Making DBCO-GalNAc to Conjugate to Azide Oligos via Click Chemistry

Click chemistry is attractive in forming GalNAc oligo conjugates due to its nature of simplicity and efficiency in bridging two parts of molecules. Using click chemistry, GalNAc moieties can be incorporated site-specifically at any position on an oligonucleotide site with azide substitutions. So the GalNAc building block described such as G010 and G011 can conjugate to oligos under copper mediated conditions to form tri-antennary GalNAc oligo conjugates, provided oligo molecules have a linker with either triple azide groups or triple terminal alkynes groups.

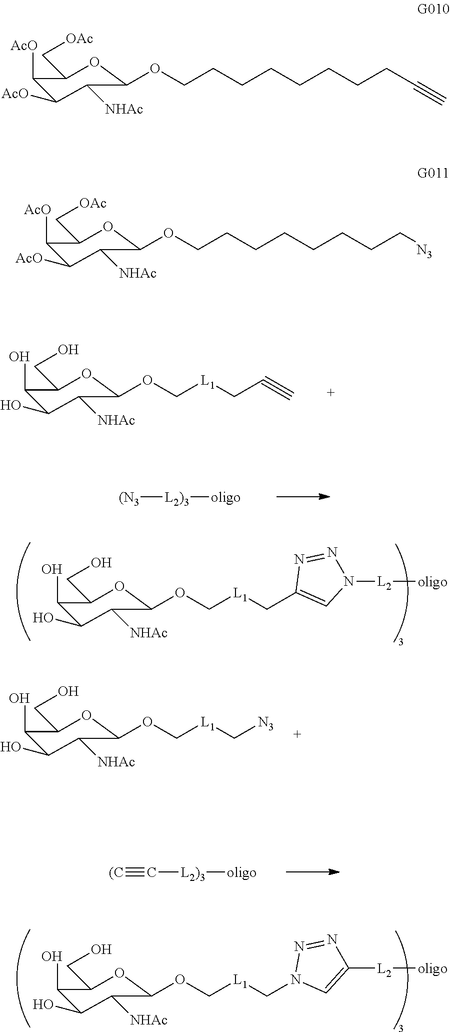

### Example 2-1

### Synthesis of G010

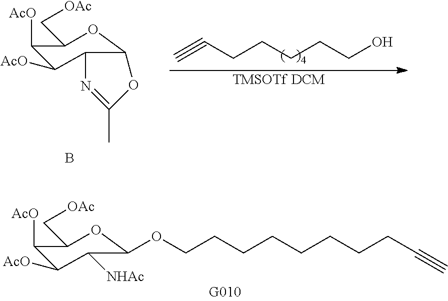

To a solution of compound B (5 g, 15.19 mmol) and decat-9-yn-1-ol (3.41 g, 30.37 mmol) in 100 mL of DCM was added TMSOTf (3.38 g, 15.19 mmol) dropwise with stirring at 0° C. The resulting solution was stirred at room temperature for 16 h. The reaction was quenched with H₂O (100 mL) and extracted with DCM (100 mL×3). The organic layer was concentrated. The organic layer was dried over anhydrous sodium sulfate, filtered, and concentrated under reduced pressure. The residue was purified by flash chromatography on reverse phase silica gel (ACN/H2O=5%-95%, 214 nm, 30 min) to afford compound C8-D (3.8 g, 83% yield) as a yellow solid. MS Calcd.: 483; MS Found: 484 [M+H]⁺. 1H NMR (400 MHz, DMSO-d6) δ: 7.80 (d, J=9.2 Hz, 1H), 5.21 (d, J=4.0 Hz, 1H), 4.97 (d, J=7.6 Hz, 1H), 4.48 (d, J=8.4 Hz, 1H), 4.04-4.01 (m, 3H), 3.90-3.83 (m, 1H), 3.72-3.67 (m, 1H), 3.44-3.38 (m, 1H), 2.71 (t, J=2.8 Hz, 1H), 2.16-2.10 (m, 2H), 2.00 (s, 3H), 1.89 (s, 3H), 1.77 (s, 3H), 1.46-1.41 (m, 4H), 1.35-1.32 (m, 2H), 1.25 (br, 6H)).

### Example 2-2

### Synthesis of Compound G011

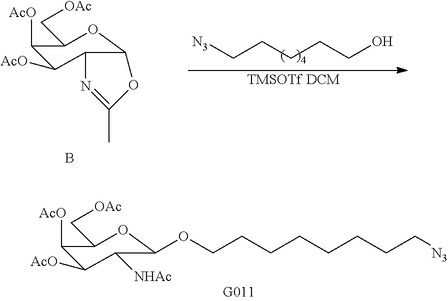

To a solution of B (4 g, 12.1 mmol) and 8-azidooctan-1-ol (3.1 g, 18.1 mmol) in dichloromethane (50 mL) was added trimethylsilyl trifluoromethanesulfonate (0.8 g, 3.6 mmol) dropwise at 0° C. under N₂. The resulting solution was stirred for 2 h at room temperature. The reaction was quenched by the addition of 100 mL ice/water and extracted with DCM (100 mL×3). The combined organic layer was washed with water and brine, dried over anhydrous sodium sulfate, filtered, and concentrated. The residue was purified by silica gel column chromatography (DCM/MeOH=100/1 to 20/1) to give compound G011 (2.5 g, 41.7%) as a light-yellow oil. LC-MS: Calcd: 500.2; Found: 501.1 [M+H⁺].

### Example 3

### Syntheses of GalNAc Phosphoramidite (That Can Be Used Directly on Automated RNA/DNA Synthesizer)

Through well-documented reactions such as (a) amide coupling, (b) nucleophilic substitution, (c) reductive amidation, (d) Heck reaction, or (e) click reaction, the GalNAc building blocks were converted to GalNAc-containing 1,2-diol and 1,3-diol which can be subsequently converted into dimethoxytrityl- (DMTr-) and phosphoramidite containing reagents (Scheme 1) that are suitable to be used in oligonucleotide synthesizers (Table 4).

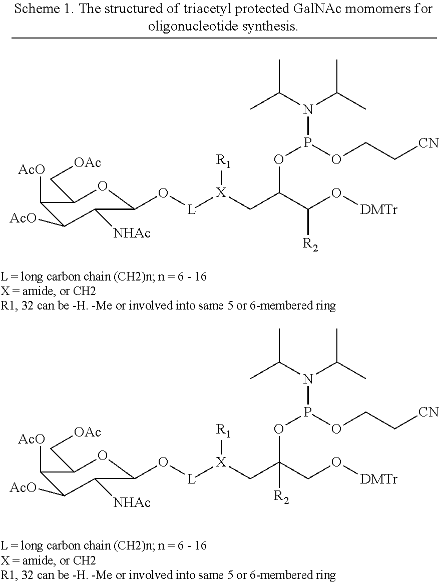

TABLE 4
GalNAc monomeric phosphoramidites suitable to be applied in oligonucleotide
synthesis

| | |
|---|---|
| 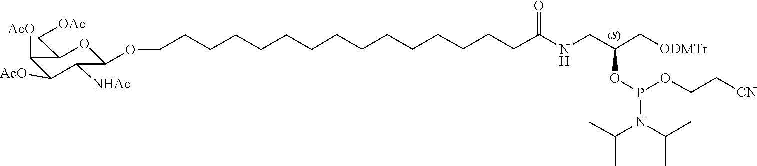 | L005 |
| 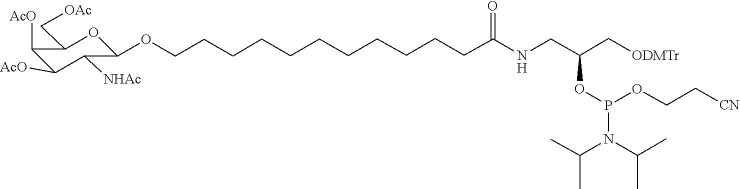 | L033 |
| 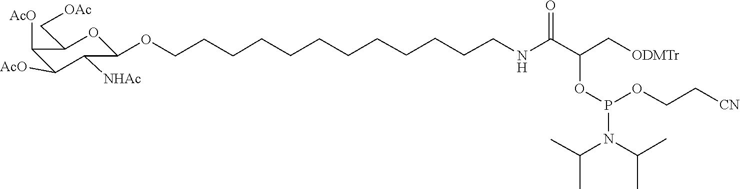 | L037 |
| 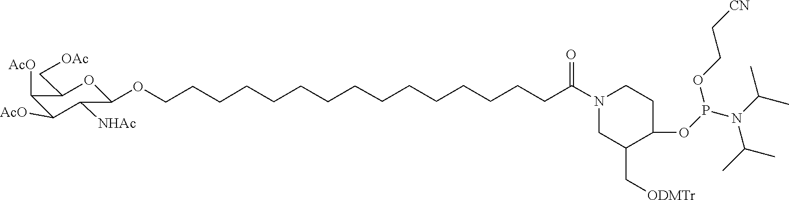 | L038 |
| 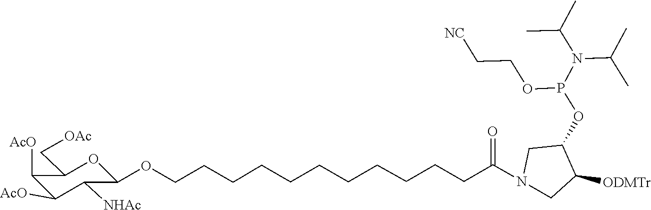 | L044 |
| 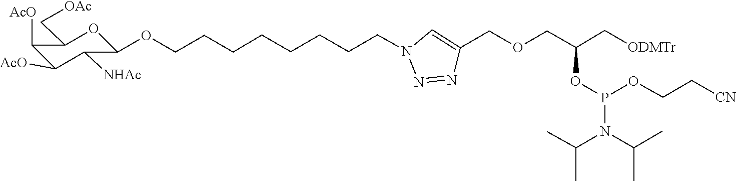 | L045 |
| 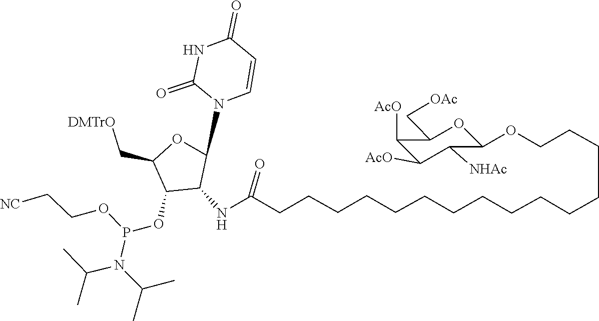 | L050 |
| 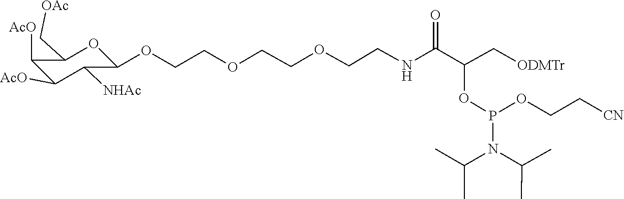 | L051 |
| 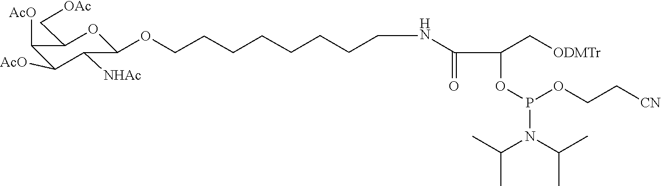 | L052 |
| 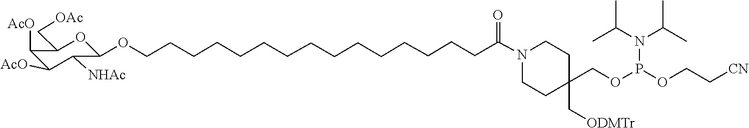 | L039 |
| 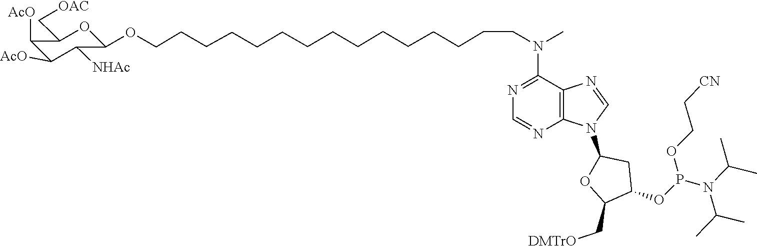 | L041 |
| 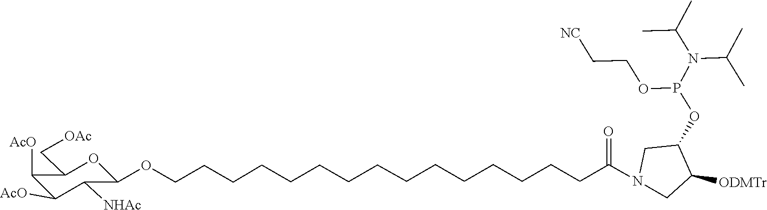 | L043 |
| 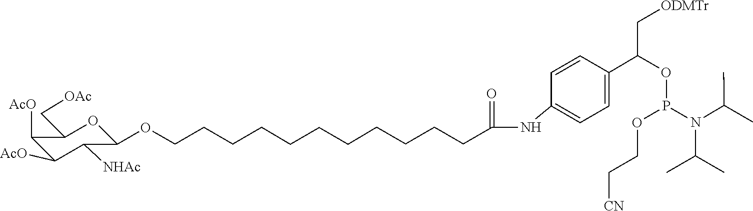 | L056 |
| 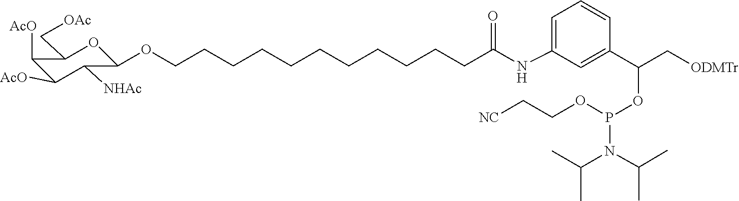 | L057 |
| 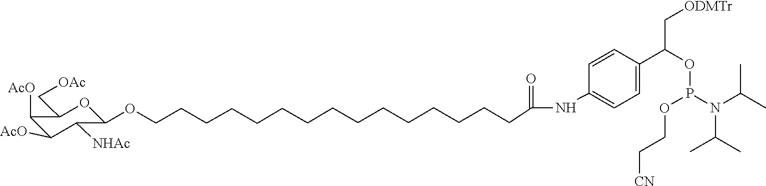 | L063 |
| 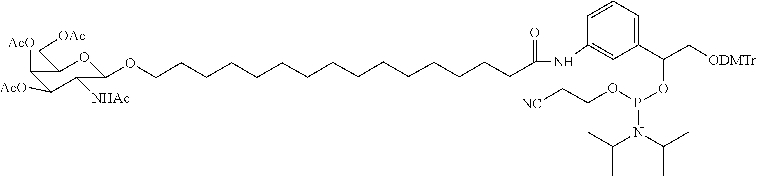 | L064 |
| 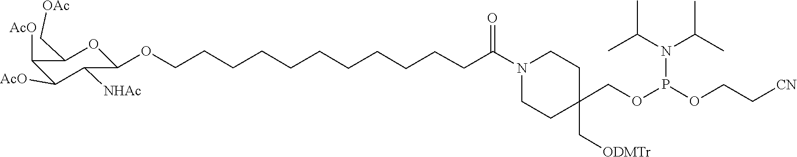 | L066 |
| 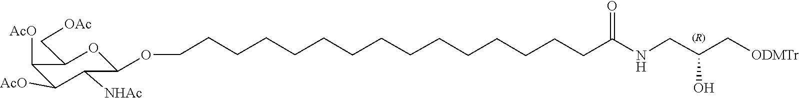 | L067 |
| 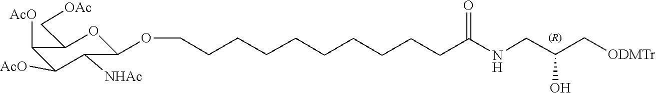 | L068 |
| 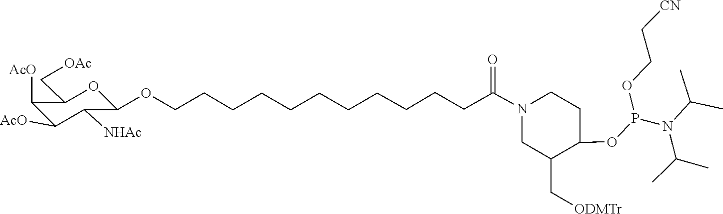 | L069 |
| 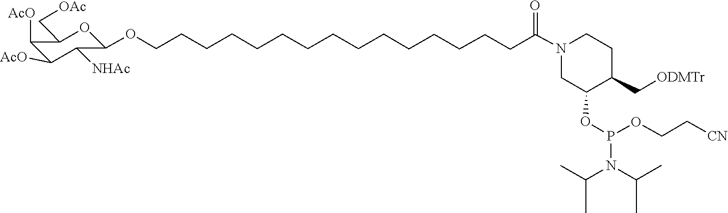 | L070 |
| 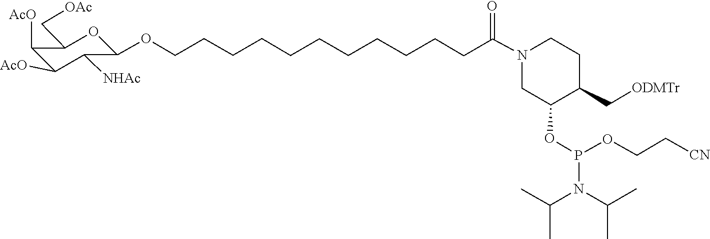 | L071 |
| 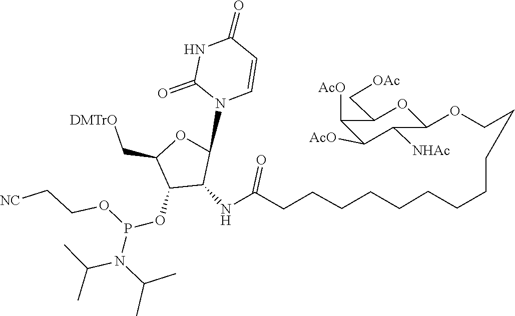 | L072 |
| 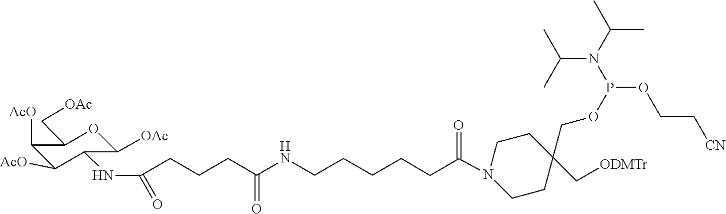 | L073 |
| 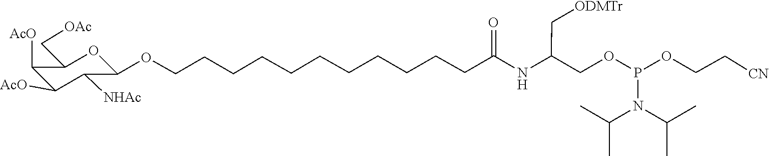 | L074 |
| 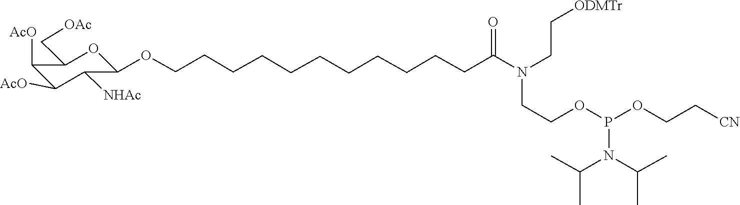 | L075 |

### Example 3-1

### Synthesis of L-005

Step 1 Synthesis of L005-diol

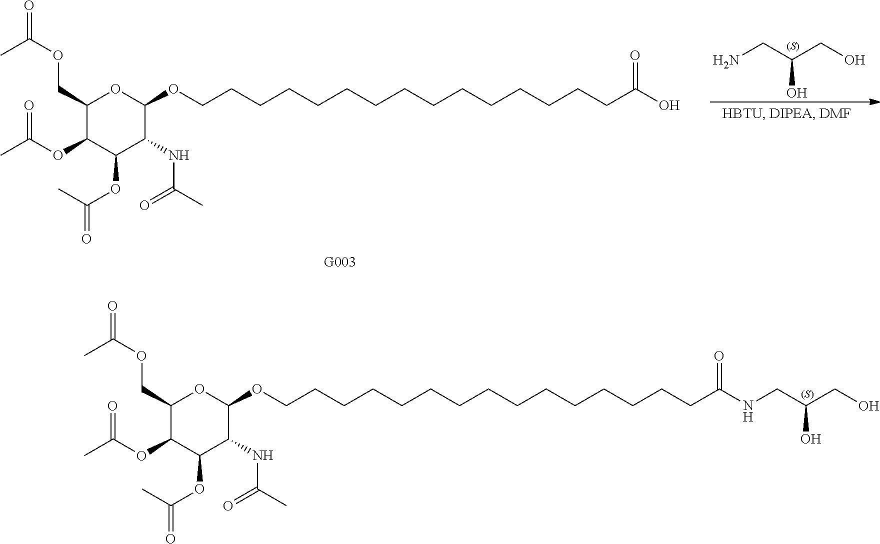

Into a 250-mL round-bottom flask, purged and maintained with an inert atmosphere of argon, was placed 16-[[(2R,3R,4R,5R,6R)-4,5-bis(acetyloxy)-6-[(acetyloxy)methyl]-3-acetamidooxan-2-yl]oxy]hexadecanoic acid G003 (6.00 g, 9.971 mmol, 1.00 equiv), dry DMF (60.00 mL), and HBTU (4.16 g, 10.968 mmol, 1.1 equiv). This was followed by the addition of DIPEA (1.42 g, 10.968 mmol, 1.1 equiv) at rt. The resulting solution was stirred for 1 hr at room temperature. To this was added 3-aminopropane-1,2-diol (1.09 g, 11.965 mmol, 1.2 equiv) at 25° C. The resulting solution was stirred for 2 hrs at room temperature. The reaction was then quenched by the addition of 100 mL of NaHCO₃ (sat). The resulting solution was extracted with ethyl acetate (2×100 mL) and the organic layers were combined. The mixture was washed with H₂O (4×100 mL) and brine. The mixture was dried over anhydrous sodium sulfate. The resulting mixture was concentrated. The product was precipitated by the addition of diethyl ether, filtration and drying, resulting in 6.2 g (purity ˜90%) of [(2R,3R,4R,5R,6R)-3,4-bis(acetyloxy)-6-[(15-[[(2S)-2,3-dihydroxypropyl]-carbamoyl]pentadecyl)oxy]-5-acetamidooxan-2-yl]methyl acetate as a white solid. LC-MS: [M+H]⁺ 675.

### Step 2 Synthesis of L005-OH

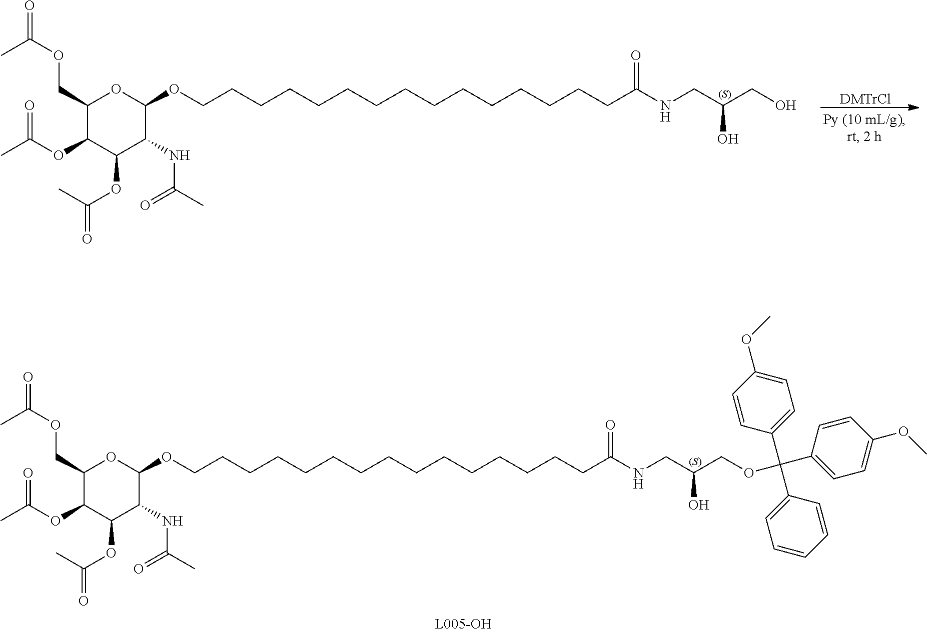

Into a 25-mL round-bottom flask, purged and maintained with an inert atmosphere of argon, was placed [(2R,3R,4R,5R,6R)-3,4-bis(acetyloxy)-6-[(15-[[(2S)-2,3-dihydroxypropyl]carbamoyl]pentadecyl)oxy]-5-acetamidooxan-2-yl]methyl acetate (1 g, 1.482 mmol, 1.00 equiv) in dry pyridine (10 mL). This was followed by the addition of 1-[chloro(4-methoxyphenyl)phenylmethyl]-4-methoxybenzene (903.78 mg, 2.667 mmol, 1.80 equiv) at 0° C. The resulting solution was stirred for 2 hr at room temperature. The resulting mixture was concentrated. The reaction was then quenched by the addition of 100 mL of water. The resulting solution was extracted with ethyl acetate (3×100 mL) and the organic layers were combined. and dried over anhydrous sodium sulfate. The solids were filtered out and the mixture was concentrated. The crude product was purified by flash-prep-HPLC with the following conditions on a CombiFlash-1 column: C18 silica gel; mobile phase, ACN/H₂O=30/70 increasing to ACN/H₂O=95/5 within 30 min. This resulted in 634 mg (43.78%) of [(2R,3R,4R,5R,6R)-3,4-bis(acetyloxy)-6-[(15-[[(2S)-3-[bis(4-methoxyphenyl) (phenyl)methoxy]-2-hydroxypropyl]carbamoyl]pentadecyl)oxy]-5-acetamidooxan-2-yl]methyl acetate as a white solid. ¹H NMR (300 MHz, DMSO-d₆) δ 7.83 (d, J=9.2 Hz, 1H), 7.66 (s, 1H), 7.42 (d, J=7.7 Hz, 2H), 7.37-7.17 (m, 7H), 6.95-6.85 (m, 4H), 5.23 (d, J=3.3 Hz, 1H), 4.98 (q, J=4.2 Hz, 2H), 4.50 (d, J=8.4 Hz, 1H), 4.04 (s, 3H), 3.88 (d, J=9.7 Hz, 1H), 3.75 (t, J=1.5 Hz, 8H), 3.42 (d, J=9.6 Hz, 1H), 3.35-3.20 (m, 1H), 3.08-2.78 (m, 3H), 2.12 (d, J=1.1 Hz, 3H), 2.08-1.96 (m, 5H), 1.91 (d, J=1.1 Hz, 3H), 1.82-1.71 (m, 3H), 1.44 (s, 4H), 1.23 (d, J=8.4 Hz, 22H) ppm.

### Step 3 Synthesis of L005

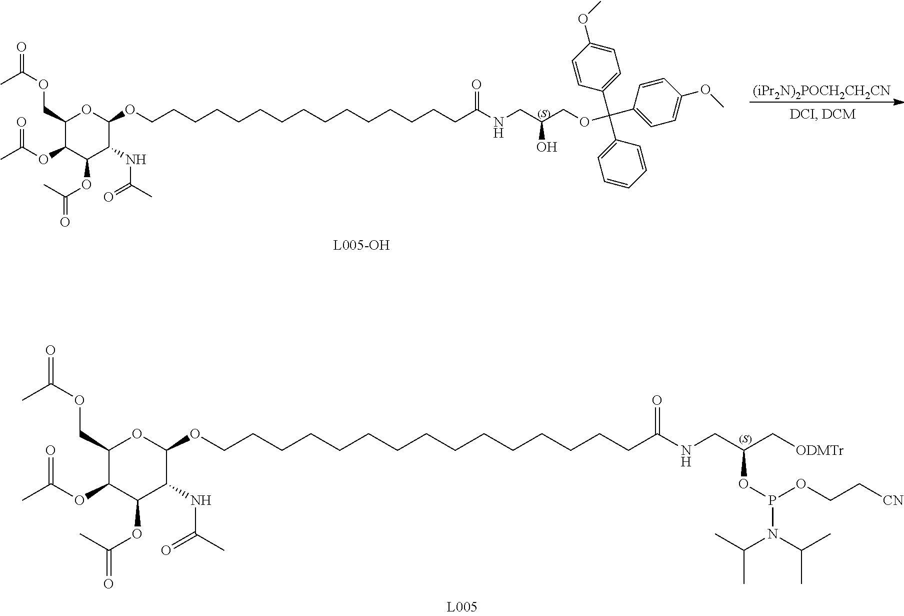

Into a 50-mL round-bottom flask, purged and maintained with an inert atmosphere of argon, was placed 3-(didiisopropylaminophosphoryl)propanenitrile (771.12 mg, 2.558 mmol, 2.50 eq.), and dry DCM (2.00 mL). This was followed by the addition of DCI (144.90 mg, 1.228 mmol, 1.20 equiv) at 0° C. The resulting solution was stirred for 10 min at 0° C. To this was added a solution of [(2R,3R,4R,5R,6R)-3,4-bis(acetyloxy)-6-[(15-[[(2S)-3-[bis(4-methoxyphenyl)(phenyl)methoxy]-2-hydroxypropyl]-carbamoyl]pentadecyl)oxy]-5-acetamidooxan-2-yl]methyl acetate (1.00 g, 1.023 mmol, 1.00 equiv) in dry DCM (4 mL) dropwise with stirring at 0° C. The resulting solution was stirred for 1 hr at room temperature. The reaction was then quenched by the addition of 50 mL of NaHCO₃ (sat. cool). The resulting solution was extracted with dichloromethane (2×100 mL) and the organic layers were combined. The resulting mixture was washed with H₂O and brine. The mixture was dried over anhydrous sodium sulfate. The solids were filtered out and the mixture was concentrated. The crude product was purified by flash-prep-HPLC with the following conditions on a CombiFlash-1 column: C18 silica gel; mobile phase, ACN/H₂O (0.1% NH₃.H₂O)=50/50 increasing to ACN/H₂O=100 within 40 min, then ACN/H₂O=100 for 20 min; detector, 220 nm/254 nm. This resulted in 612 mg (50.79%, stored under Ar with 4 Å MS, −70° C.) of [(2R,3R,4R,5R,6R)-3,4-bis(acetyloxy)-6-[(15-[[(2S)-3-[bis(4-methoxyphenyl)(phenyl)methoxy]-2-[[(2-cyanoethoxy)-(diisopropylamino)phosphanyl]oxy]propyl]carbamoyl]pentadecyl)oxy]-5-acetamidooxan-acetamidooxan-2-yl]methyl acetate as a white solid. ¹H NMR (300 MHz, DMSO-d₆) δ 7.83 (d, J=9.3 Hz, 1H), 7.66 (s, 1H), 7.43 (d, J=7.6 Hz, 2H), 7.28 (qd, J=11.4, 9.4, 6.6 Hz, 7H), 6.88 (dd, J=8.6, 4.6 Hz, 4H), 5.23 (d, J=3.3 Hz, 1H), 4.99 (dd, J=11.3, 3.3 Hz, 1H), 4.50 (d, J=8.5 Hz, 1H), 4.04 (s, 4H), 3.96-3.77 (m, 2H), 3.77-3.63 (m, 11H), 3.43 (dd, J=10.1, 6.0 Hz, 1H), 3.19 (s, 2H), 3.03 (d, J=6.2 Hz, 1H), 2.79 (t, J=6.0 Hz, 1H), 2.65 (t, J=5.9 Hz, 1H), 2.12 (s, 3H), 2.01 (s, 6H), 1.91 (s,2H), 1.78 (s, 3H), 1.50-1.36 (m, 4H), 1.28-1.10 (m, 31H), 1.03 (d, J=6.6 Hz, 3H) ppm. ³¹P NMR (300 MHz, DMSO-d₆) δ 148.41, 147.94 ppm.

### Example 3-2

### Synthesis of L045

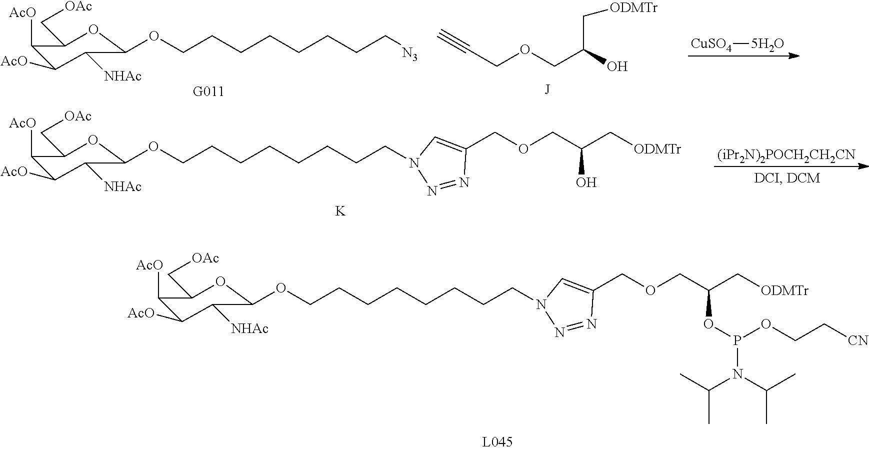

### Step 1: Synthesis of Compound K:

To a solution of compound G011 (1.67 g, 3.33 mmol) in t-BuOH (15 mL) was added compound J (1.58 g, 3.61 mmol). To this stirred solution was added CuSO₄.5H₂O (164 mg, 0.66 mmol) and sodium ascorbate (328 mg, 1.66 mmol) in water (15 mL). After stirring for 4 h at 35° C., the reaction mixture was extracted with EtOAc (20 mL×2). The organic layer was dried over Na₂SO₄, filtered and concentrated to give the residue which was purified by silica gel column chromatography (DCM/MeOH=100/1 to 20/1) to provide the pure compound K (1.1 g, yield 33.3%) as a white solid. LC-MS: m/z Calcd: 932.4; Found: 955.4 [M+Na]⁺. ¹H NMR (DMSO-d₆, 400 MHz), δ 8.00 (s,1H), 7.80 (d, J=9.2 Hz, 1H), 7.38 (d, J=7.6 Hz, 2H), 7.30-7.18 (m, 7H), 6.87 (d, J=8.8 Hz, 4H), 5.21 (d, J=2.8 Hz, 1H), 4.96 (dd, J=11.6 Hz, 3.6 Hz, 1H), 4.88 (d, J=5.6 Hz, 2H), 4.50-4.46 (m, 3H), 4.29 (t, J=7.2 Hz, 2H), 4.03-4.01 (m, 3H), 3.85 (dd, J=20.4 Hz, 9.6 Hz, 1H), 3.77-3.65 (m, 8H), 3.52 (dd, J=10.0 Hz, 4.4 Hz, 1H), 3.45-3.36 (m, 2H), 2.91 (d, J=5.2 Hz, 2H), 2.09 (s, 3H), 1.98 (s, 3H), 1.88 (s, 3H), 1.77-1.75 (m, 5H), 1.43-1.41 (m, 2H), 1.21 (s, 6H).

### Step 2: Synthesis of Compound L045

Into a 50-mL round-bottom flask, purged and maintained with an inert atmosphere of argon, was placed 3-(didiisopropylaminophosphoryl)propanenitrile (90 mg, 21.50 eq.) and dry DCM (2.00 mL). This was followed by the addition of DCI (78 mg, 3.0 equiv) at 0° C. The resulting solution was stirred for 10 min at 0° C. To this was added a solution of K (186 mg, 1.0 equiv) in dry DCM (1 mL) dropwise with stirring at 0° C. The resulting solution was stirred for 1 hr at room temperature. The reaction mixture was concentrated and purified on a silica gel column using hexanes/ethyl acetate elution with 1% triethylamine modulation. This resulted in 172 mg L045 as a white semi-solid. ¹H NMR (DMSO-d₆, 400 MHz), δ 7.95 (d, J=9 Hz, 1H), 7.80 (d, J=9 Hz, 1H), 7.d (m, 2H), 7.30-7.18 (m, 7H), 6.8 (m, 4H), 5.21 (d, J=3 Hz, 1H), 4.96 (dd, J=12 Hz, 4 Hz, 1H), 4.50-4.46 (m, 3H), 4.29 (m, 2H), 4.0 (m, 5H), 3.85 (m, 1H), 3.77-3.45 (m, 13H), 3.45-3.36 (m, 2H), 2.91 (m, 1H), 2.75-2.55 (m, 2H), 2.09 (s, 3H), 1.98 (s, 3H), 1.88 (s, 3H), 1.77 (s, 3H), 1.43-1.41 (m, 2H), 1.25-0.95 (m, 18H). ³¹P NMR (300 MHz, DMSO-d₆) δ 148.50, 147.96 ppm.

### Example 4

### Expediting the Syntheses of GalNAc Monomers by Simplifying Linker Structure

Certain phosphoramidite building blocks such as L035 can be synthesized in four steps from common intermediates in high yield. The process is high-yielding and scalable for large-scale synthesis. A representative method and synthetic protocol is given below:

### Example 4-1

### Synthesis of L-035

### Step 1: Synthesis of Alkene F is Similar to the Synthesis of G009 Described in Example 1.

Step 2: Synthesis of L035-diol (G)

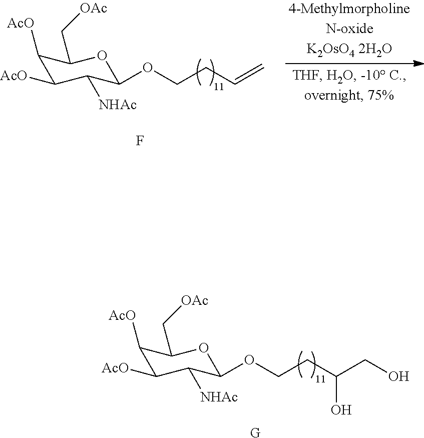

F (0.93 g, 1.72 mmol) was dissolved in THF/H₂O (12.23 mL/1.58 mL) and cooled to −10° C. before the addition of 4-methylmorpholine N-oxide hydrate (0.678 g, 5.02 mmol) and K₂OsO₄.2H₂O (0.027 g, 0.076 mmol). The resulting mixture was stirred at −10° C. overnight before addition of Na₂S₂O₃ and further stirring for 30 minutes. The mixture was diluted with water and extracted with ethyl acetate (3×50 mL). The combined organic layers were washed with H₂O, dried over Na₂SO₄, filtered, and evaporated in vacuo to give a residue. The residue was purified on a silica gel column to yield 0.741 g (75%) of G.

### Step 3: Synthesis of L035-OH (H)

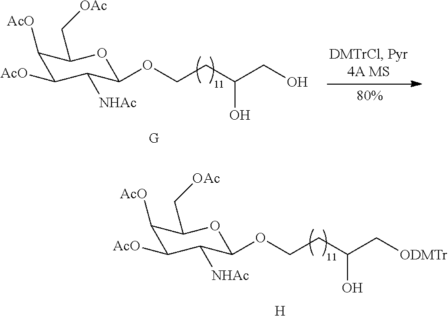

0.8 g (1.39 mmol) of G was dissolved in 7.5 mL anhydrous pyridine and stirred with 4 Å molecular sieves at ambient temperature. DMTrCl (0.6 g, 1.77 mmol) was added in one batch. The resulting mixture was stirred overnight before being diluted with DCM (30 mL). The pyridine was removed by repeatedly washing the organic layer with saturated CuSO₄ and the organic layer was dried over Na₂SO₄, filtered, and evaporated in vacuo. The residue was purified on a silica gel column to yield0.976 g (80%) of H. [M+Na]⁺=900.2. ¹H NMR (400 MHz, CDCl3) δ 7.46-7.38 (m, 2H), 7.36 -7.16 (m, 7H), 6.87-6.78 (m, 4H), 5.42 (d, J=8.6 Hz, 1H), 5.39-5.27 (m, 2H), 4.71 (d, J=8.3 Hz, 1H), 4.22-4.07 (m, 2H), 3.97-3.82 (m, 3H), 3.79 (s, 6H), 3.75 (s, 1H), 3.47 (dt, J=9.7, 6.9 Hz, 1H), 3.16 (dd, J=9.3, 3.3 Hz, 1H), 3.00 (dd, J=9.4, 7.6 Hz, 1H), 2.34 (d, J=3.5 Hz, 1H), 2.14 (s, 3H), 2.07-1.92 (m, 9H), 1.61-1.51 (m, 1H), 1.39 (d, J=17.7 Hz, 3H), 1.36-1.20 (m, 19H), 1.11 (s, 1H) ppm.

### Step 4: Synthesis of L035

L035 phosphoramidite was synthesized in four steps similar to the synthetic protocol described in Example 2.

### Example 5

### Automatic Synthesis of Tri-Antennary Formation by the GalNAc Monomer

Synthesis of tri-antennary 5′-GalNAc-conjugated oligonucleotides was carried out on ABI394 or K&A-H8 DNA/RNA synthesizer. The synthesis was carried out on a 1 μmole scale on NittoPhaseHL UnyLinker solid support. Trichloroacetic acid (3% by volume) in toluene was used for cleaving the 4,4′-dimethoxytrityl (DMTr) groups from the 5′-hydroxyl group of the nucleotide. 4,5-Dicyanoimidazole in the presence of N-methylimidazole was used as the activator during the coupling step. During the coupling step, 10-50 molar equivalents of 0.05 M phosphoramidite solution (2′-deoxy, 2′-O-methoxyethyl, and Locked nucleosides) and a flow ratio of 1:1 (v/v) of phosphoramidite solution to activator solution was used. Phosphoramidite and activator solutions were prepared using low-water acetonitrile (water content <30 ppm) and were dried further by the addition of molecular sieve packets. Phosphorothioate linkages were introduced by oxidation of phosphite triesters with 0.05 M xanthane hydride solution in pyridine. A solution of iodine in pyridine/water was used during the oxidation step to obtain phosphodiester linkages. Unreacted hydroxyl groups were capped by using N-methylimidazole/pyridine/acetonitrile and acetic anhydride/acetonitrile delivered in a 1:1 (v/v) flow ratio. At the end of the synthesis, the support-bound oligonucleotide was treated with a solution of triethylamine/acetonitrile (1:1, v/v) to remove acrylonitrile formed during deprotection of the cyanoethyl group from the phosphorothioate triester. Automated DNA/RNA synthesizer manufacturer recommended protocols of reagent delivery volumes and contact times were followed as detailed in Table 5. Subsequently, the support-bound oligonucleotide was incubated with concentrated aqueous ammonium hydroxide at 55° C. for approximately 15 h to complete the cleavage from the solid support, eliminate the UnyLinker molecules to liberate the 3′-hydroxy groups of the oligonucleotides, and deprotect the nucleobase-protecting groups. After allowing the crude mixture to cool to room temperature, it was filtered and the solid support was rinsed with purified water and collected. The crude product in ammonia solution was concentrated and purified by gel electrophoresis and/or reversed phase HPLC to obtain pure oligonucleotide-GalNAc conjugate. In general, the conjugate purity was found to be over 85% by anion-exchange HPLC.

TABLE 5
Reaction parameters for 1 μmol scale synthesis on the synthesizer

| | | | Reaction |
|---|---|---|---|
| | | Volume | time |
| Reagent | Components | (uL) | (s) |
| CAP-A | 90% ACN, 10% Acetic anhydride | 1600 | 36 |
| CAP-B | 76% ACN, 14% N-Methyl | 1600 | 36 |
| | imidazole, 10% pyridine | | |
| DEBLOCK | 3% Trichloroacetic acid, | 1200 | 44 |
| | Methylene chloride | | |
| Oxidizer | 0.05M iodine, 10% water, 20% | 1720 | 19 |
| | pyridine, 70% tetrahydrofuran | | |
| Activation | 0.25M 5-Tethiotetrazol CAN, | 480 | 50-300 |
| | 1-Methyl imidazole | | |
| Sulfurization | 0.048M xanthane hydride, 40% | 1720 | 319 |
| | pyridine, 60%ACN | | |

### Example 6

### Incorporate GalNAc Conjugate Moiety to Antisense Sequences

The oligonucleotide selected for GalNAc conjugate moiety can be single strand antisense oligos or double-stranded siRNAs wherein multi-antennary GalNAc can be conjugated at the 3′- or 5′-termini. As an example, we have conjugated GalNAc to a 13-mer antisense oligonucleotide targeted to Apo B100 mRNA at the 5′-terminal and studied target knockdown in C57BL/6 mice.

The following is the 13-mer gapmer sequence (Nucleic Acids Research, 2018, 46, 5366-5380) used in our studies: 5′-[L]ₙ[Sp]ₘ[+G]*[+mC]*[A]*[T]*[T]*[G]*[G]*[T]*[A]*[T]*[+T]*[+mC]*[+A]-3′, in which [L] is a GalNAc containing ligand, n=1-4; [Sp] is an optional spacer, either —(CH₂)ₙ— chain, wherein n=3-12, or —(OCH₂CH₂)ₘ—O—, wherein m=1-3, between GalNAc conjugate moiety and ApoB antisense sequence, m=0-2; [+N] is locked nucleic acid and [N] is deoxyribonucleoside, and * is phosphorothioate linkage.

Methods: similar methods as those described in example 4 were used to make the oligonucleotide-GalNAc conjugates described. The obtained crude oligonucleotide-GalNAc conjugate products were further purified by RP-HPLC or PAGE to yield pure products whose molecular integrity was confirmed by mass spectrometry. Endotoxin levels were checked prior to animal studies.

Using the general methods described in Examples 4 and 5, the following antisense sequences oligonucleotide-GalNAc conjugates were synthesized. The structure and characterization data of each antisense sequences oligonucleotide-GalNAc conjugates are shown in Table 6.

TABLE 6
Structure of ASO-GalNAc conjugates

| | Gel or | | |
|---|---|---|---|
| | HPLC | | |
| | purity | mass | mass |
| Structure of antisense sequences oligonucleotide-GalNAc conjugates | analysis | (Calculated) | (found) |
| 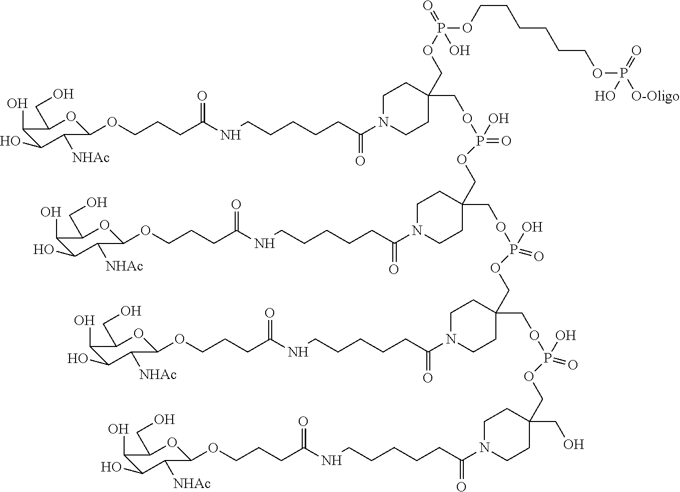 B006 | Single band on gel | 6944.1 | 6944.0 |
| 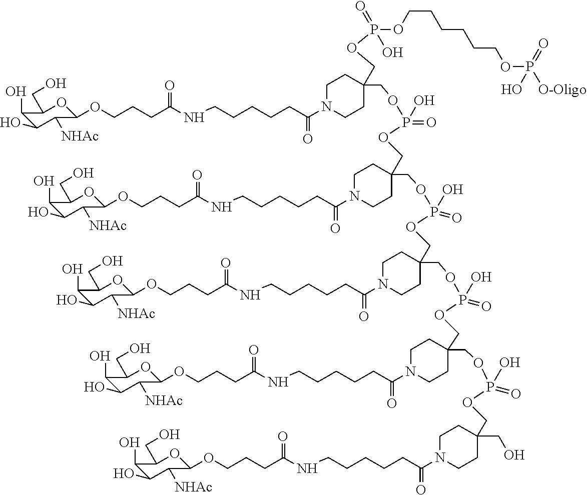 B007 | Single band on gel | 7553.7 | 7553.8 |
| 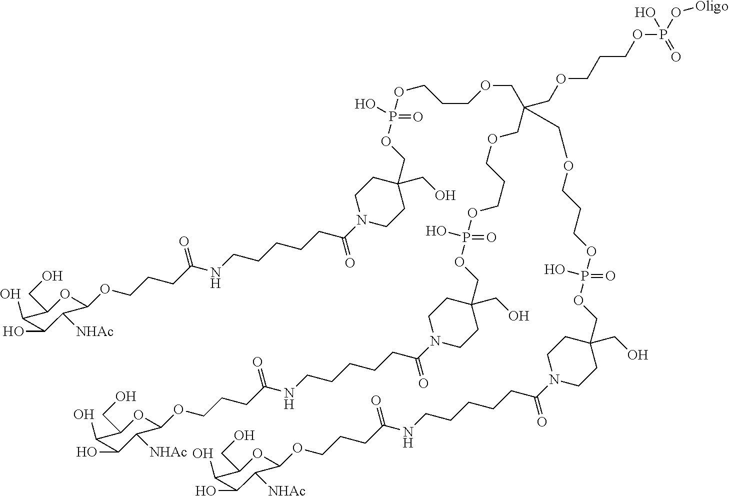 B008 | Single band on gel | 6584.5 | 6584.6 |
| 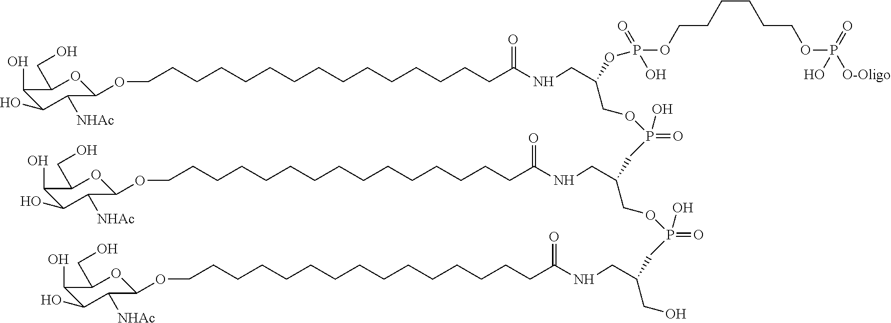 B009 | Single band on gel | 6336.6 | 6338.1 |
| 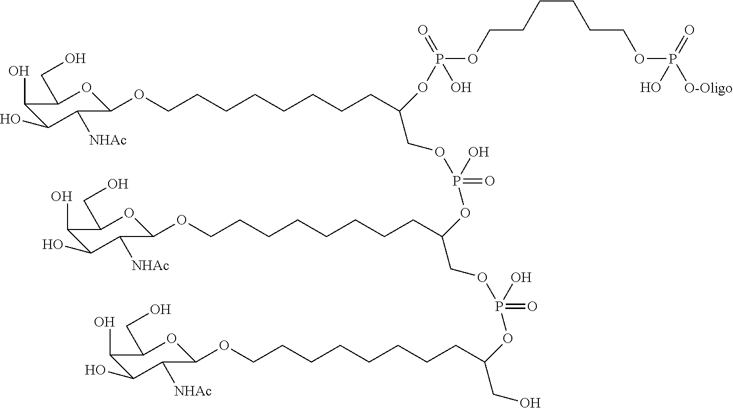 B011 | Single band gel | 5871.2 | 5871.4 |
| 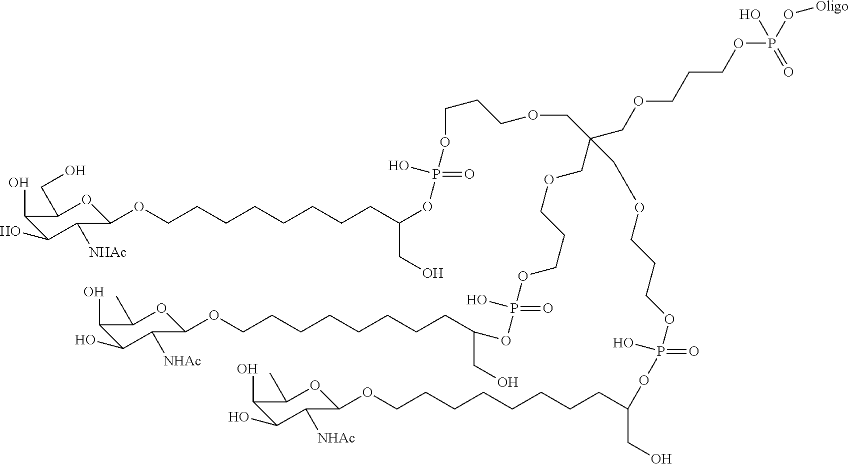 | Single band on gel | 6121.3 | 6121.7 |
| 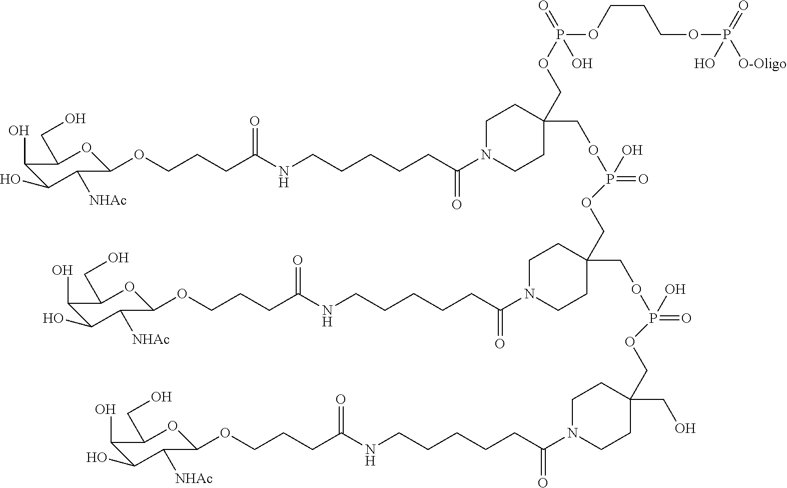 B013 | Single band on gel | 6292.4 | 6291.6 |
| 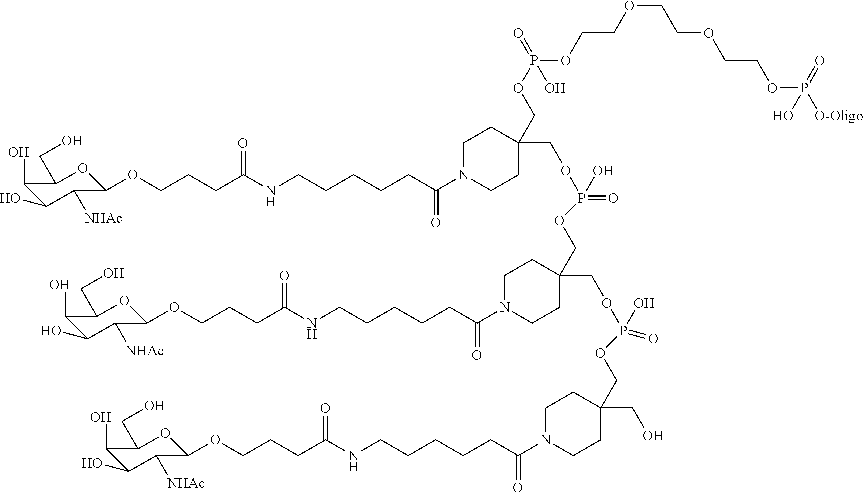 | HPLC >95.0% | 6366.5 | 6365.8 |
| 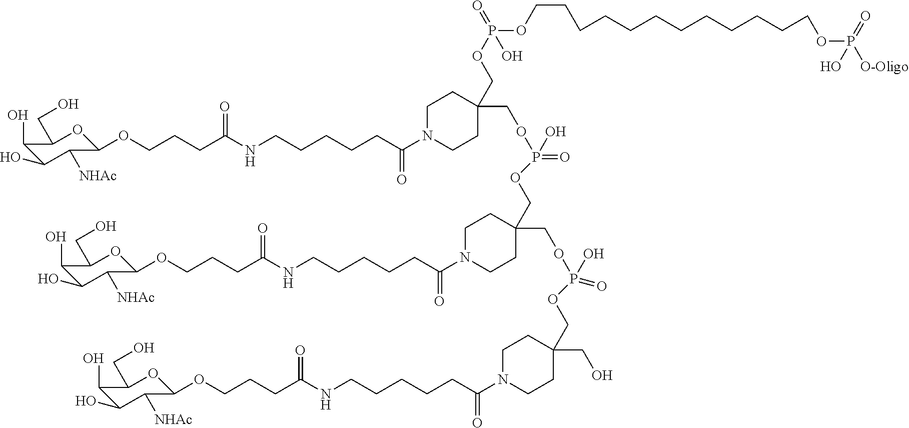 B015 | Single band on gel | 6418.6 | 6417.9 |
| 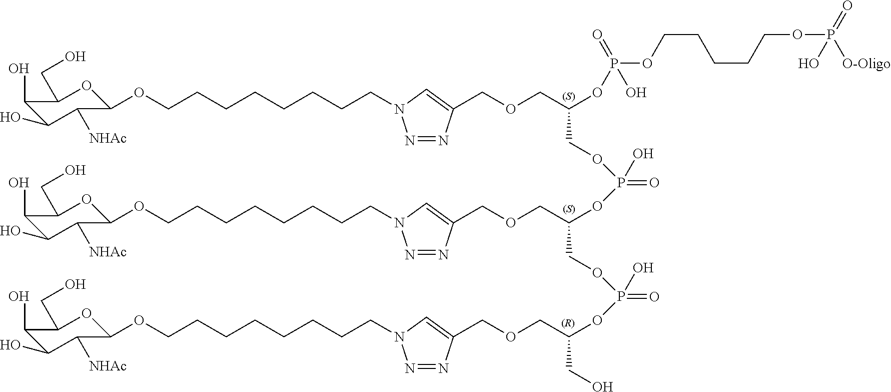 | Single band on gel | 6204.4 | 6204.6 |
| 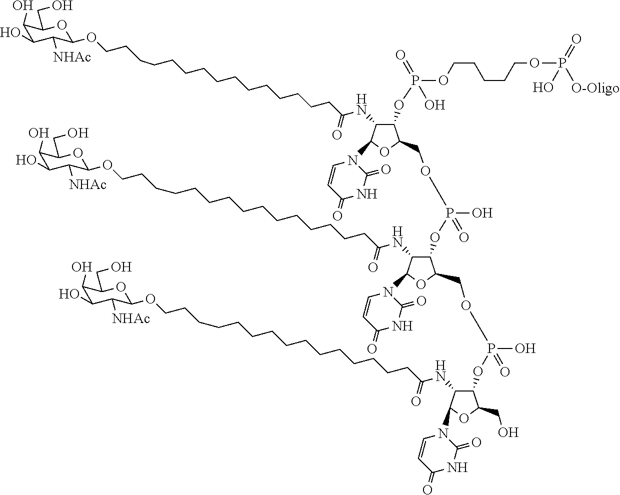 | Single band on gel | 6792.7 | 6793.4 |
| 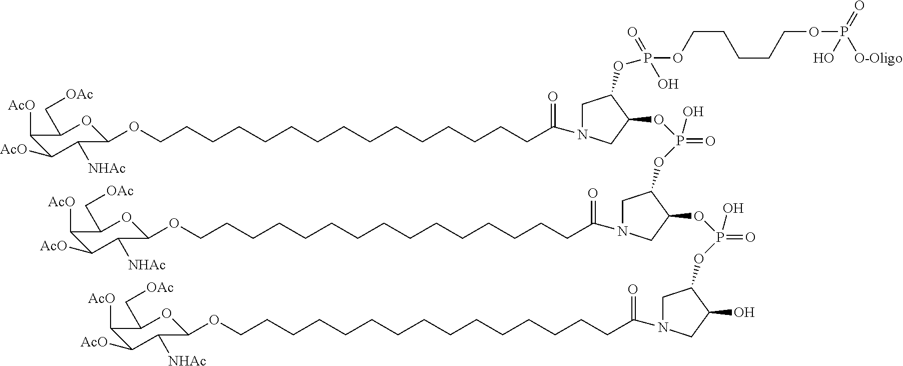 | Single band on gel | 6372.6 | 6372.8 |
| 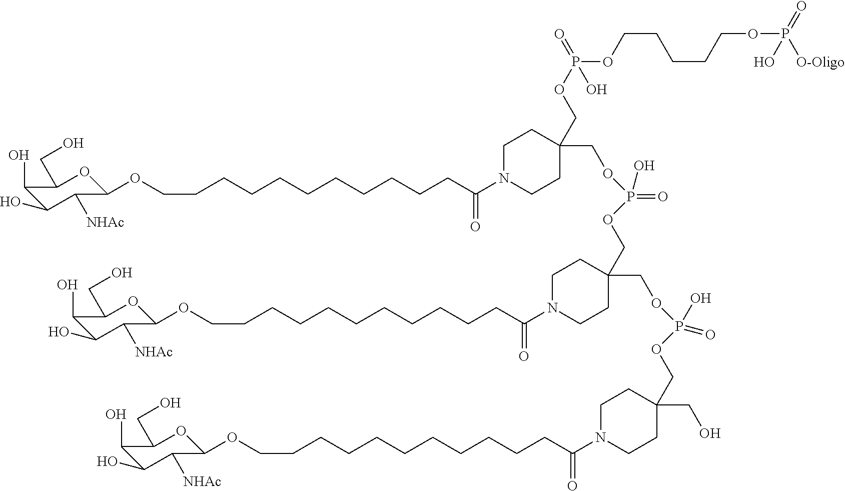 | Single band on gel | 6331.7 | 6330.7 |
| 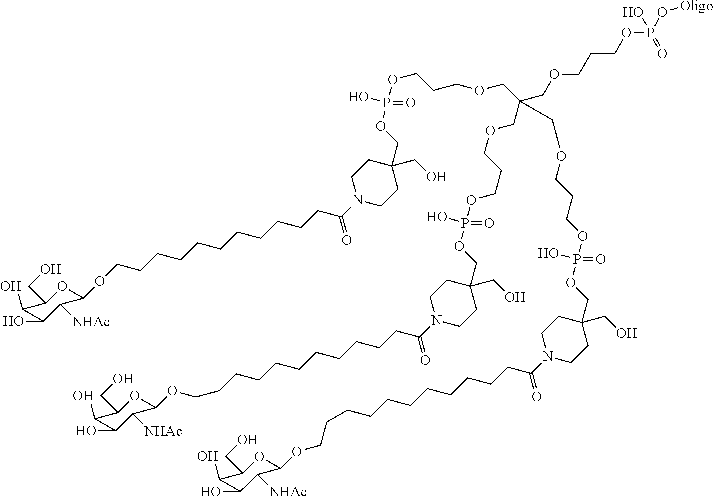 | Single band on gel | 6581.7 | 6580.5 |
| 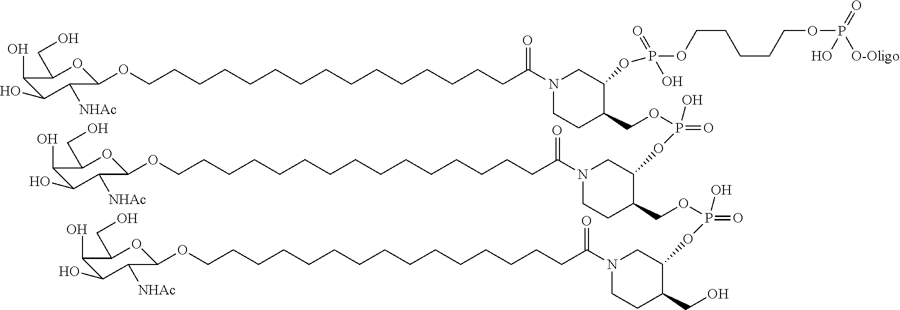 | Single band on gel | 6456.7 | 6457.5 |
| 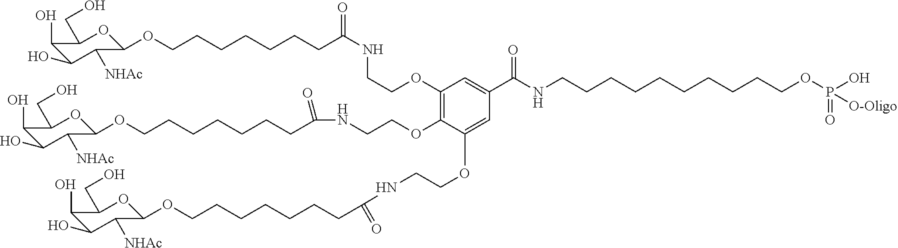 | Single band on gel | 5877.3 | 5877.4 |
| 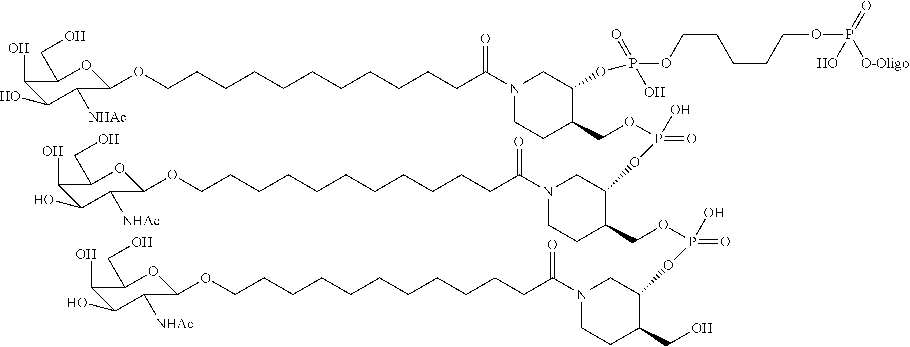 | Single band on gel | 6288.5 | 6288.2 |
| 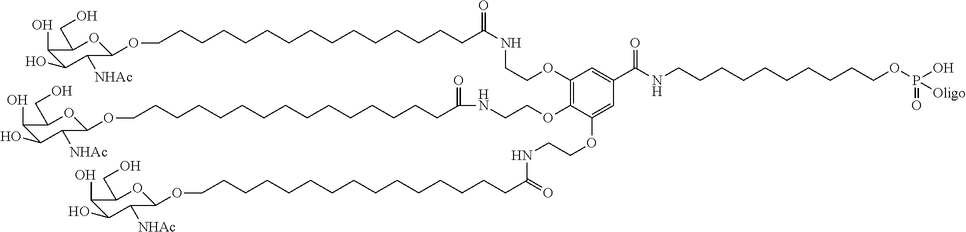 | HPLC 76% | 6157.6 | 6158.3 |
| 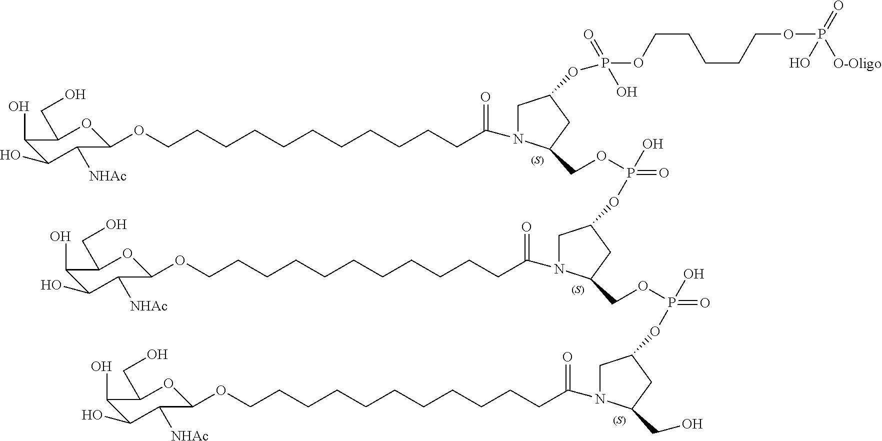 | HPLC >95.0% | 6246.5 | 6246.6 |
| 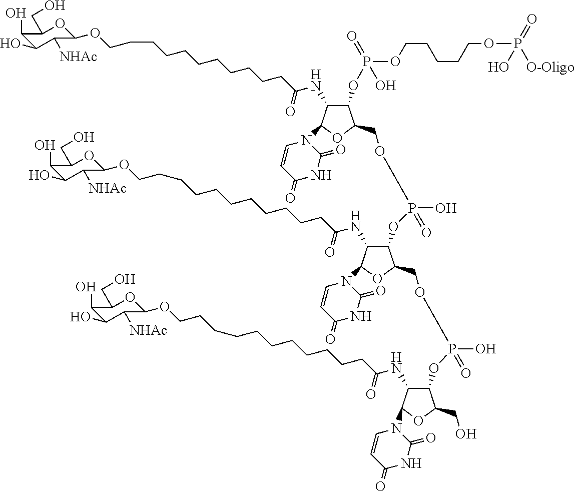 | HPLC >95.0% | 6624.5 | 6625.1 |

Note:
“Oligo” = 5′-[+G]*[+mC]*[A]*[T]*[T]*[G]*[G]*[T]*[A]*[T]*[+T]*[+mC]*[+A]

### Example 7

### Screening GalNAc Monomers Through GalNAc Conjugated ApoB Antisense Oligos

The oligonucleotide-GalNAc conjugates for the studies were prepared as described in example 5 and 6 and formulated in PBS before studies. Mice were grouped based on BW at day −7, five mice/group. Mice were dosed once at day 0 at two different dose levels (high, 60 nmoles/kg and low, 20 nmoles/kg) and were subsequently bled to monitor plasma Apo B100 (ApoB) protein levels at day 3 and day 6. The study was terminated on the last observation day, or humane endpoint whichever came first. Blood of ˜50 uL/mouse/timepoint via tail or retro orbital bleeding were collected into an EDTA coated tube. Sample is centrifuged for 10 minutes at 1,000-2,000×g in a refrigerated centrifuge. Following centrifugation, the resulting supernatant (plasma) was immediately transferred into a clean labelled polypropylene tube and stored at −80° C. until use.

Plasma ApoB level was determined by commercial ELISA kit (AbCam #ab230932). The assay was performed according to manufacturer's instructions. Plasma samples were tested at 5000-fold dilutions in duplicate. ApoB results were reported either as ug/mL or normalized as a percentage of the initial level of ApoB before dosing of oligonucleotide-GalNAc conjugates. The comparison between compounds was used to elucidate structure-activity relationships (SAR) and the comparison to tri-antennary positive control compound B005 was used as a standard compound.

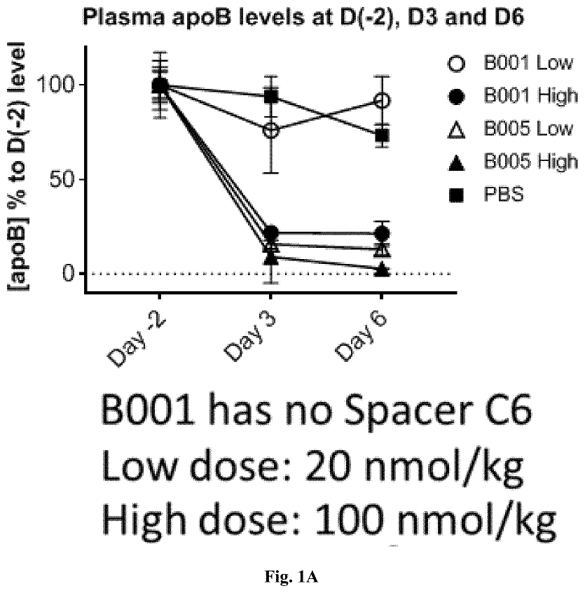

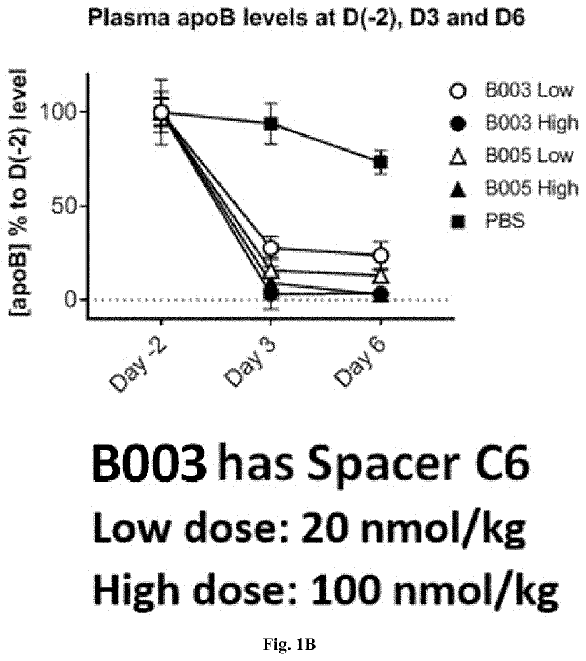

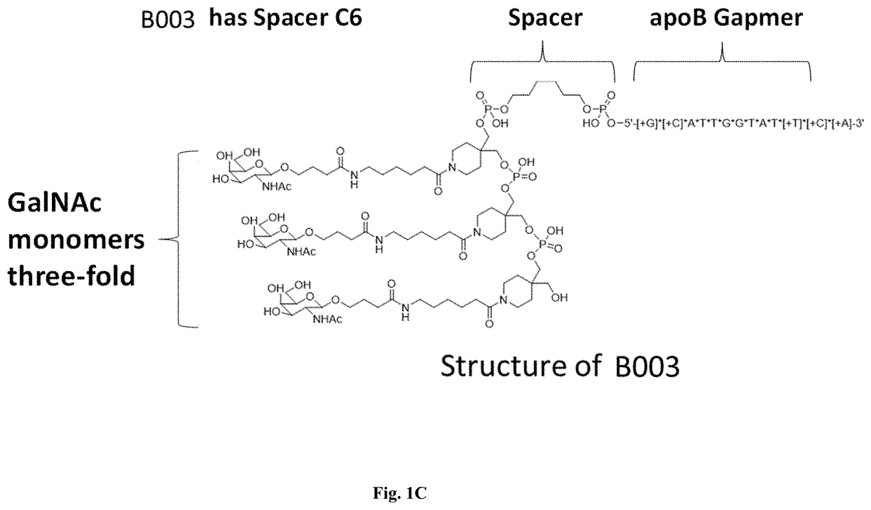

B001 is a tri-antennary GalNAc gapmer without a spacer between GalNAc cluster and gapmer. B003 has a 1,6-hexanediol spacer (Spacer, e.g. C6) between GalNAc and gapmer through a phosphodiester linkage. The in vivo studies demonstrated the superior activity of B003 over B001 at both 100 nmol/kg and 20 nmol/kg dosing levels, indicating a spacer is required between the GalNAc moiety and antisense moiety (FIG. 1A-1C).

### Example 8

### Using GalNAc Monomers to Form Branched GalNAc Clusters

The monomeric GalNAc phosphoramidites were effective in forming various multi-antennary GalNAc clusters using standard DNA/RNA synthesizers using branch-enabling building blocks such as doubler or trebler. For example, apart from the linear form of tri-antennary GalNAc described in example 6, we synthesized trebler tri-antennary GalNAc oligos on a synthesizer.

Sequences 5′-[L]3[Trebler][+G]*[+mC]*[A]*[T]*[T]*[G]*[G]*[T]*[A]*[T]*[+T]*[+mC]*[+A]-3′, in which [L] is a GalNAc ligand:

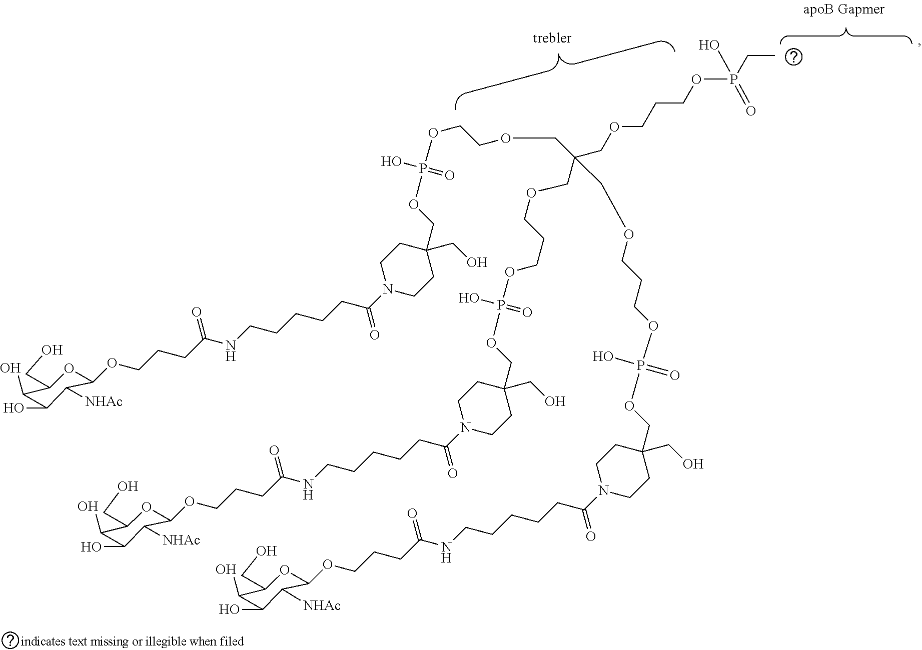 [SMILES: CCP(=O)(O)OCCCOCC(COCCCOP(=O)(O)OCC1(CO)CCN(C(=O)CCCCCNC(=O)CCCOC2OC(CO)C(O)C(O)C2NC(C)=O)CC1)(COCCCOP(=O)(O)OCC1(CO)CCN(C(=O)CCCCCNC(=O)CCCOC2OC(CO)C(O)C(O)C2NC(C)=O)CC1)COCCOP(=O)(O)OCC1(CO)CCN(C(=O)CCCCCNC(=O)CCCOC2OC(CO)C(O)C(O)C2NC(C)=O)CC1]

and [Trebler] is the building block with following chemical structure:

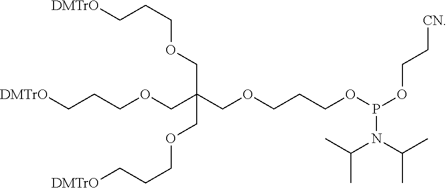

[+N] is locked nucleic acid, [N] is deoxyribonucleoside, and * is phosphorothioate linkage. The sequence is synthesized and evaluated in mice using the protocols described in examples 4 and 6. The resultant compounds demonstrated excellent plasma ApoB reduction in comparison to positive control compound B005. To reach multiplicity higher than three, we could form tetra-antennary GalNAc clusters through a doubler of doubler, thus providing multiple forms of GalNAc clusters for lead candidate selections:

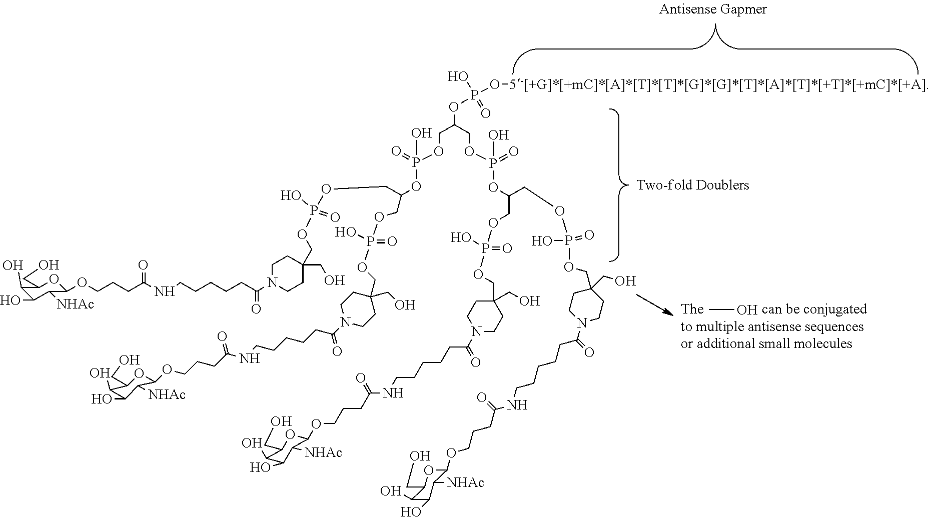

In both cases, the exposed 5′-OH ends resulting from oligonucleotide conjugate synthesis could be conjugated to other modalities to modulate the oligonucleotide conjugate properties. Those modalities include, but are not limited to, other antisense sequences, or small molecules that can modulate endosome-escaping reagents to help oligonucleotide conjugates enter the cytosol.

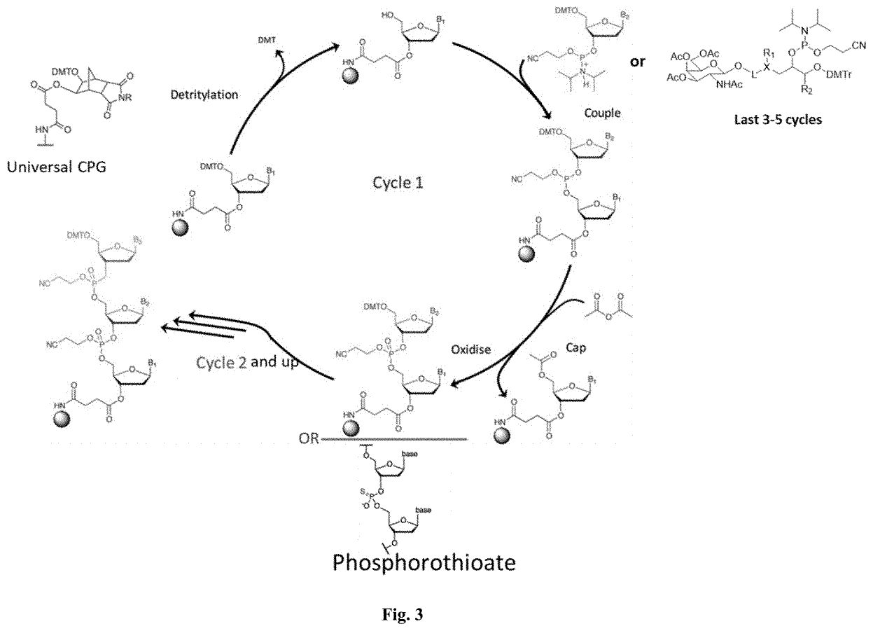

The standard synthetic cycles for oligonucleotide syntheses used on DNA/RNA synthesizers on universal linker solid support are shown in FIG. 3. After completion of oligonucleotide synthesis, the GalNAc phosphoramidites synthesized are used to conjugate to the oligonucleotides on the synthesizer.

All conjugates were purified by either PAGE or anion-exchange HPLC. The purity of final conjugates was found to be 85-95% as determined by AE-HPLC. The molecular integrity was determined by Mass Spectrophotometry and the results are shown in the above table. All conjugates were checked for endotoxin levels by Charles River's Endosafe® system via the Endosafe® LAL cartridge method prior to administration to mice for in vivo studies.

### Example 9

### Use of Long Carbon Chains in Forming GalNAc Clusters

We incorporated long carbon chains into the GalNAc clusters instead of using multiple amide groups to elongate the chain length to simplify the synthesis by reducing the number of steps and also to modulate the biophysical properties of GalNAc-oligonucleotide conjugates for optimal pharmacokinetic profiles, as shown in Scheme 2. (A, Left) GalNAc clusters in published literature use multiple amides and result in a compound that is hydrophilic overall (B. right). Long carbon chain in monomer and spacer are easier to form than multiple amide bonds and can balance the hydrophilicity of the compound.)

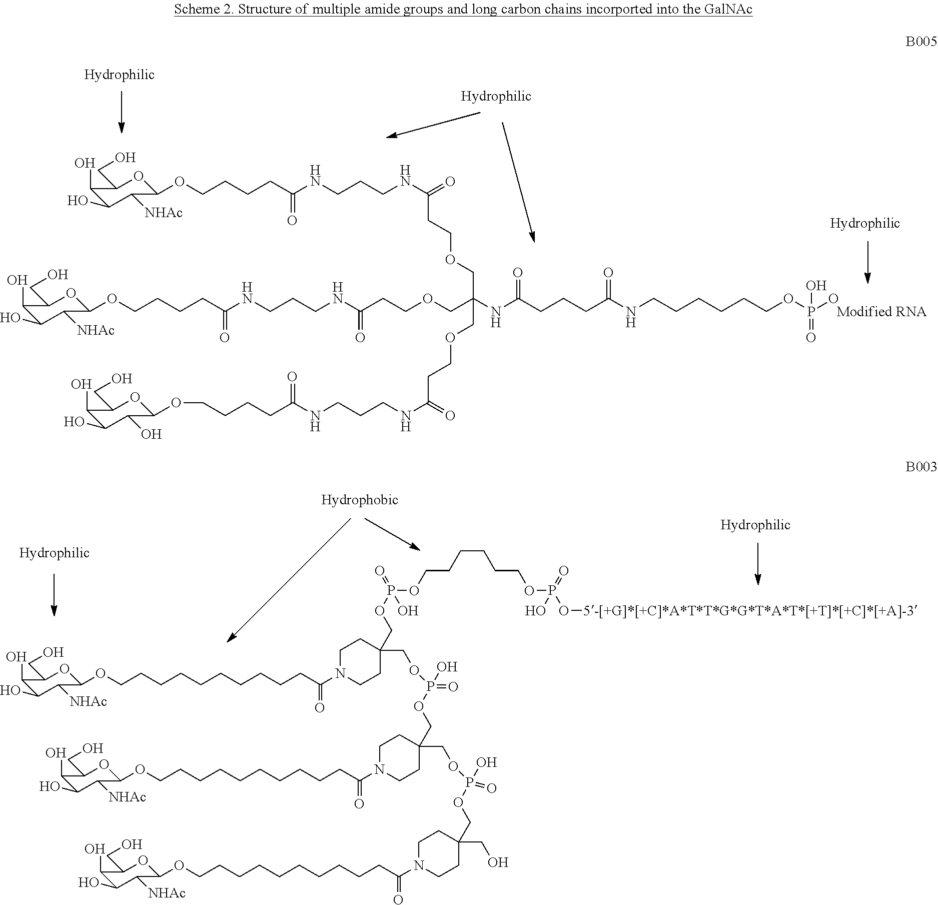

Both GalNAc moiety and oligonucleotide moiety are known to be extremely hydrophilic which is known to facilitate their renal clearance. Modulating the biophysical properties with hydrophobic carbon chains in the molecules may reduce the rate of renal clearance to allow more oligonucleotide-conjugate intake by the liver.

### Example 10

### Significant Reduction in Reaction Steps by Adopting Monomeric GalNAc Building Blocks

Through the adoption of monomeric GalNAc phosphoramidites, we significantly reduce the complexity of the synthesis of GalNAc clusters. A typical GalNAc cluster exemplified by B005 requires at least 14 steps and time-consuming synthesis before its application in oligonucleotide-conjugate synthesis (see below).

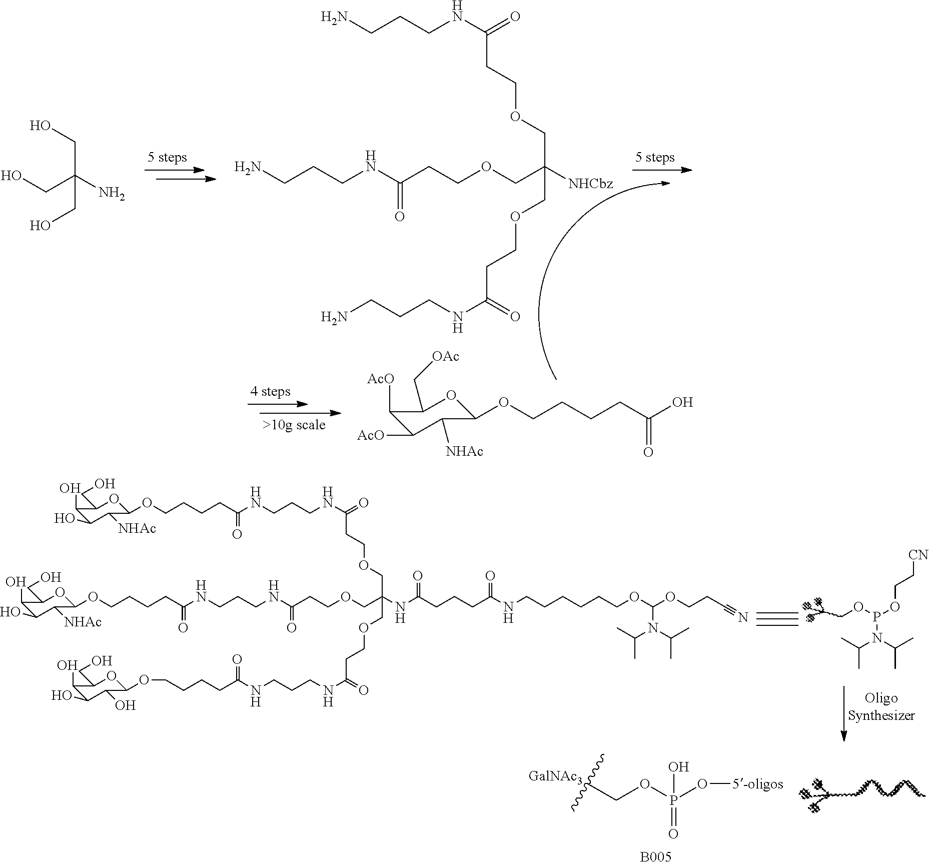

Additional detailed description of the synthesis method may be found in U.S. Pat. Nos. 8,828,956 and 9,943,604, the disclosure of which is herein incorporated by reference.

In contrast, a monomeric GalNAc phosphoramidite typically takes 8 steps from commercial starting material, or only 4 steps from a typical intermediate such as G001 (see below).

The synthesis of a new monomeric GalNAc phosphoramidite can be accomplished in a typical chemistry lab within a short time period.

We also designed and synthesized monomeric GalNAc phosphoramidites by completely avoiding amide bonds or other typical linkers to simplify the chemical synthesis. The diol moiety that is required for phosphoramidite synthesis can be effectively constructed from dihydroxylation of a terminal alkene such as G009. The diol was subsequently modified into dimethoxytrityl protecting groups (DMTr) and phosphoramidite, respectively, in as little as 4 steps in high yields (see below).

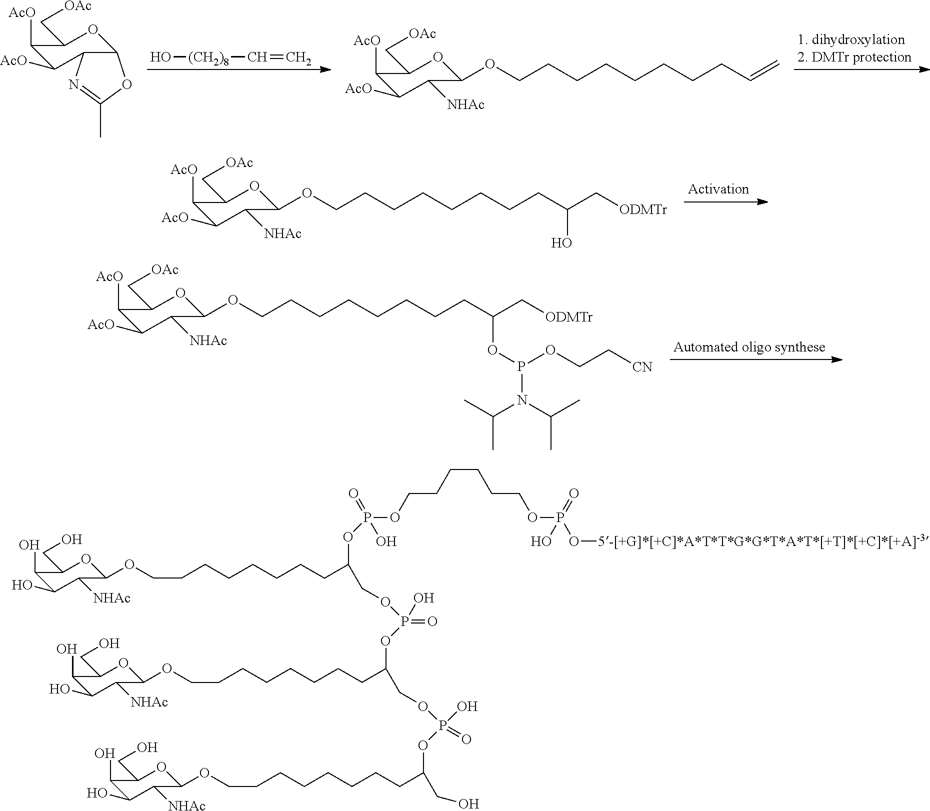

### Example 10-1

### Syntheses of L009 and Oligonucleotide Conjugation

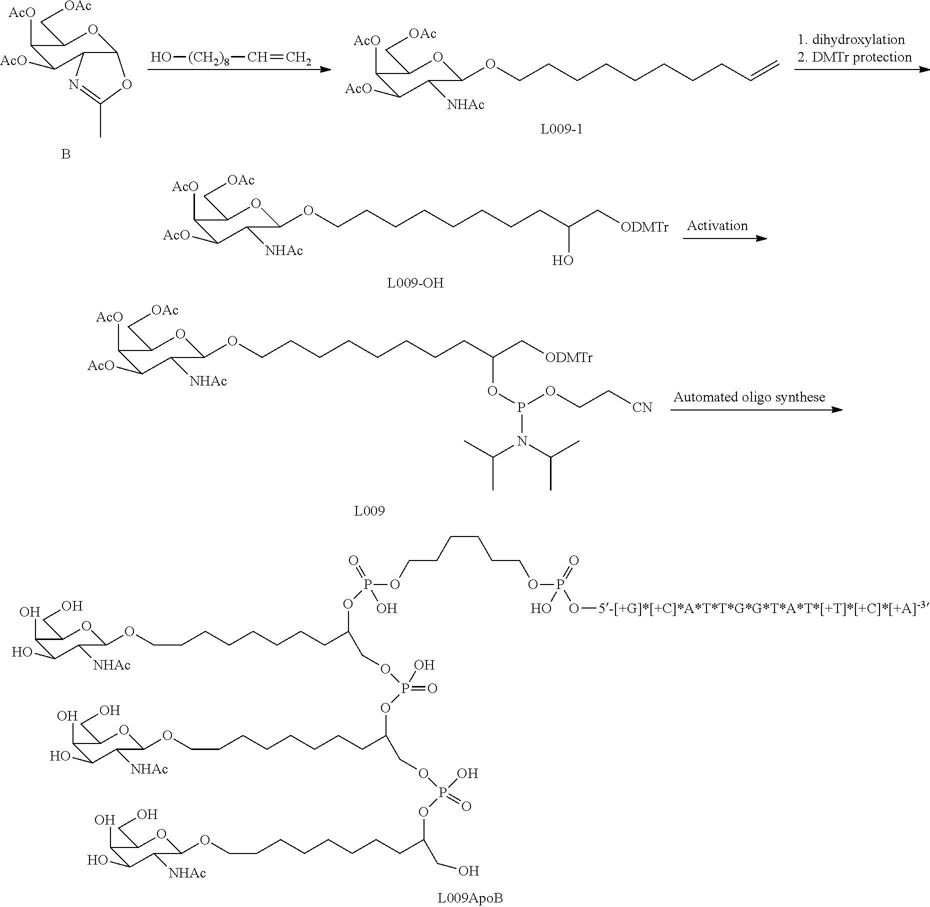

### Step 1: Synthesis of L009-1

The crude starting material B (3.3 g) and C₁₀-vinyl alcohol (1.7 g, 11 mmol) were dissolved in 20 mL of anhydrous THF. The mixture was degassed and charged with argon for three times. Under argon protection, TMSOTf (1.1 g, 0.9 mmol) was added to the mixture dropwise. After the addition, the mixture was stirred for overnight at room temperature. When the reaction was completed, the mixture was poured into cold 10% sodium bi-carbonate solution (100 mL) and the mixture was stirred for 10 min. 100 mL ethyl acetate was added to the mixture and the mixture was stirred for 10 min and the organic phase was separated, and aqueous phase was extracted by ethyl acetate (50 mL×2). The organic phase was combined, washed by brine, and then evaporated to pale yellow liquid. The residue was purified by silica gel column (PE/EA=0% to 80%) to provide the compound L009-1 as a colorless oil (2.6 g, 53.5% yield for two steps). MS Calcd: 485.3; Found 486.3 [M+H]⁺. ¹H NMR (400 MHz, CDCl₃) δ 7.8 (m, 1H), 6.8 (m, 1H), 5.2 (d, J=8.0 Hz, 1H), 5.10-4.90 (m, 3H), 4.5 (d, J=8.0 Hz, 1H), 4.10-4.00 (m, 3H), 3.90-3.60 (m, 2H), 3.40-3.50 (m, 2H), 2.14 (s, 3H), 2.07-1.92 (m, 11H), 1.6-1.5 (m, 2H), 1.36-1.20 (m, 10H) ppm.

Step 2: Synthesis of L009-1,2-diol

L009-1 (2.6 g, 5.4 mmol) was dissolved in 30 mL of THF and potassium osmate dihydrate (18 mg, 0.05 mmol) was added to the mixture. 5 mL water was added to the mixture until the potassium osmate was dissolved. The mixture was cooled to 0-10° C. in an ice bath and 4-methylmorpholine N-oxide (937 mg, 8.0 mmol) was added in several portions. After the addition, the ice bath was removed, and the mixture was stirred at room temperature for 16 hr. The mixture was poured into cold 10% sodium sulfite solution (50 ml), and ethyl acetate (50 mL) was added. The mixture was stirred for 10 min and the organic phase was separated. The aqueous phase was extracted twice by 50 mL ethyl acetate. The organic phase was combined, washed by brine, dried through anhydrous sodium sulfate, and then evaporated to obtain a pale yellow oil. This crude product was purified by silica gel column (PE/EA=0% to 100%) to obtain a pale yellow oil (2.6 g, 94.2%).

### Step 3: Synthesis of L009-OH

To a solution of L009-1,2-diol (2.6 g, 5.0 mmol) and TEA (1.5 g, 15.1 mmol) in 30 mL of anhydrous DCM, a solution of DMTr-Cl (2.1 g, 6.1 mmol) in 10 mL of anhydrous DCM was added dropwise. After addition, the mixture was stirred at room temperature for 16 hr. When the reaction was completed, 50 mL DCM was added to dilute the mixture and 50 ml brine was added. The mixture was stirred for 10 min and the organic phase was separated. The aqueous phase was extracted by DCM (50 ml). The organic phase was combined and evaporated to a yellow oil. The residue was purified by silica gel column (PE/EA=0% to 80%) to obtain L009-OH as a white vesicular solid (2.0 g, 48.4%). MS Calcd: 822.0; Found: 844.4 (M+Na⁺). ¹H NMR (400 MHz, CDCl₃) δ 7.4 (m, 2H), 7.4-7.2 (m, 7H), 6.8 (m, 4H), 5.6 (m, 1H), 5.4 (m, 1H), 4.7 (m, 1H), 4.2-4.0 (m, 2H), 4.0-3.8 (m, 3H), 3.79 (s, 6H), 3.75 (m, 1H), 3.50-3.45 (m, 1H), 3.2 (m, 1H), 3.00 (m, 1H), 2.4 (m, 1H), 2.14 (s, 3H), 2.07-1.92 (m, 9H), 1.6-1.5 (m, 2H), 1.5-1.2 (m, 14H) ppm.

### Step 4: Synthesis of L009 and Oligonucleotide Conjugation

The general method described in examples 2, 3, and 5 was used to synthesize L009 and L0009ApoB.

L009, MS calculated: 1022.2; Observed: 1022.3 (M+H⁺).

L009-ApoB antisense conjugate, MS calculated: 5871.2; found: 5871.4 (M−H⁺).

### Example 11

### Use of Multi-Antennary Branched Group in Forming GalNAc clusters

We designed and synthesized tri-antennary GalNAc clusters to compare with monomeric GalNAc for in vivo efficacy. These novel clusters feature a benzene ring or cyclen (aza-crown ether) ring to construct multi-antennary GalNAc clusters. The structures of the GalNAc phosphoramidite clusters synthesized is listed below.

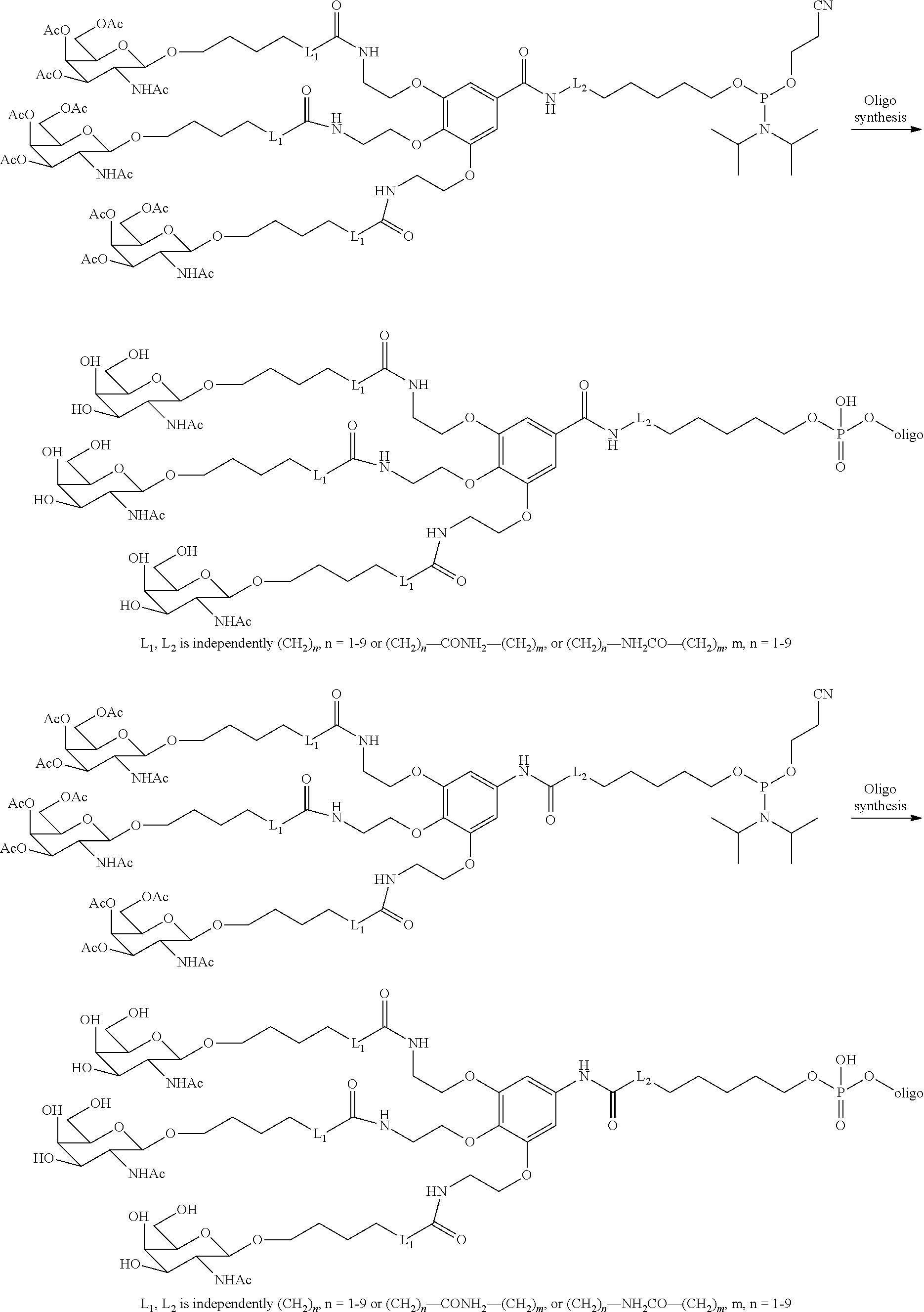

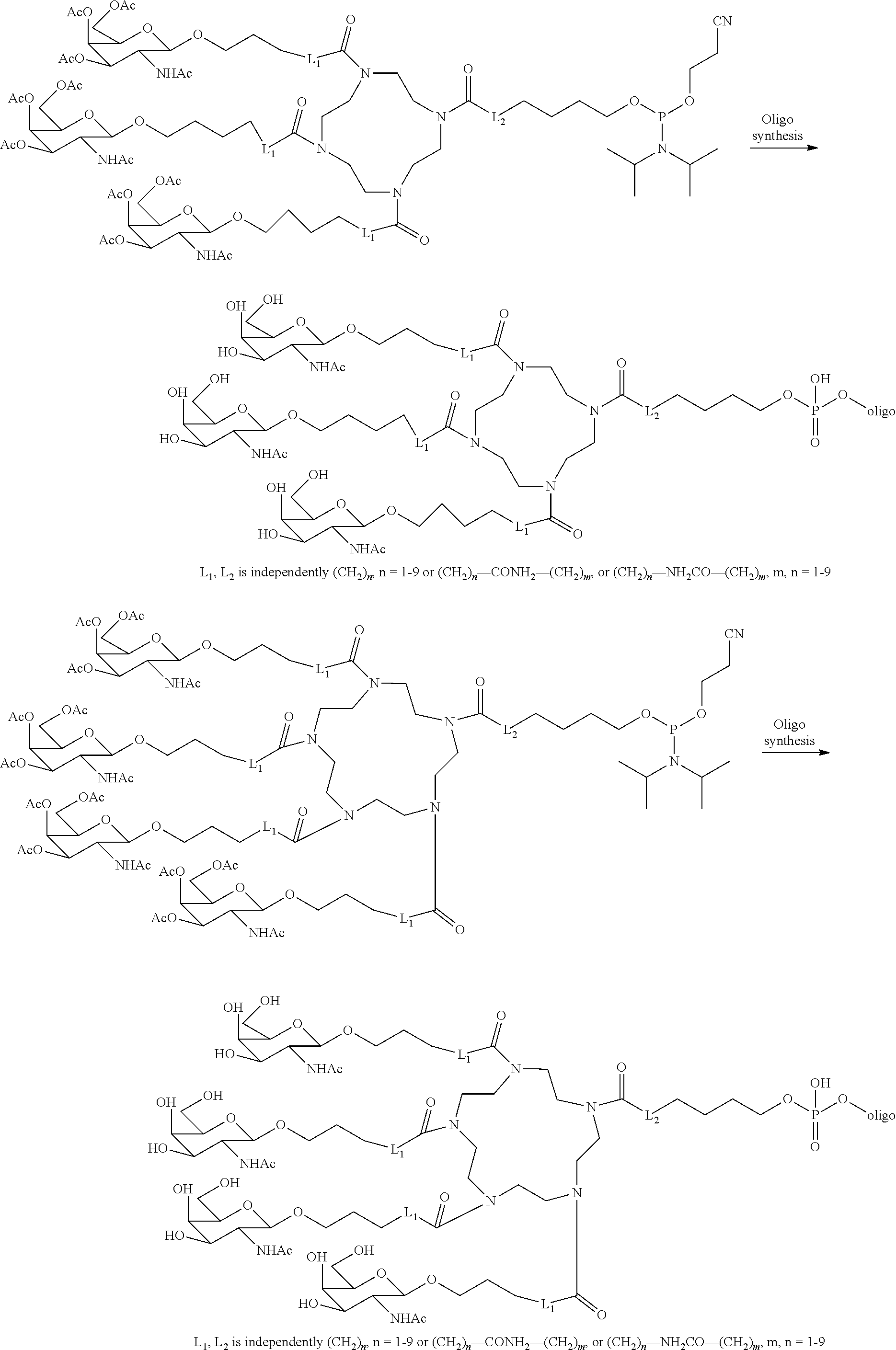

### Example 11-1

### Synthesis of L016-OH

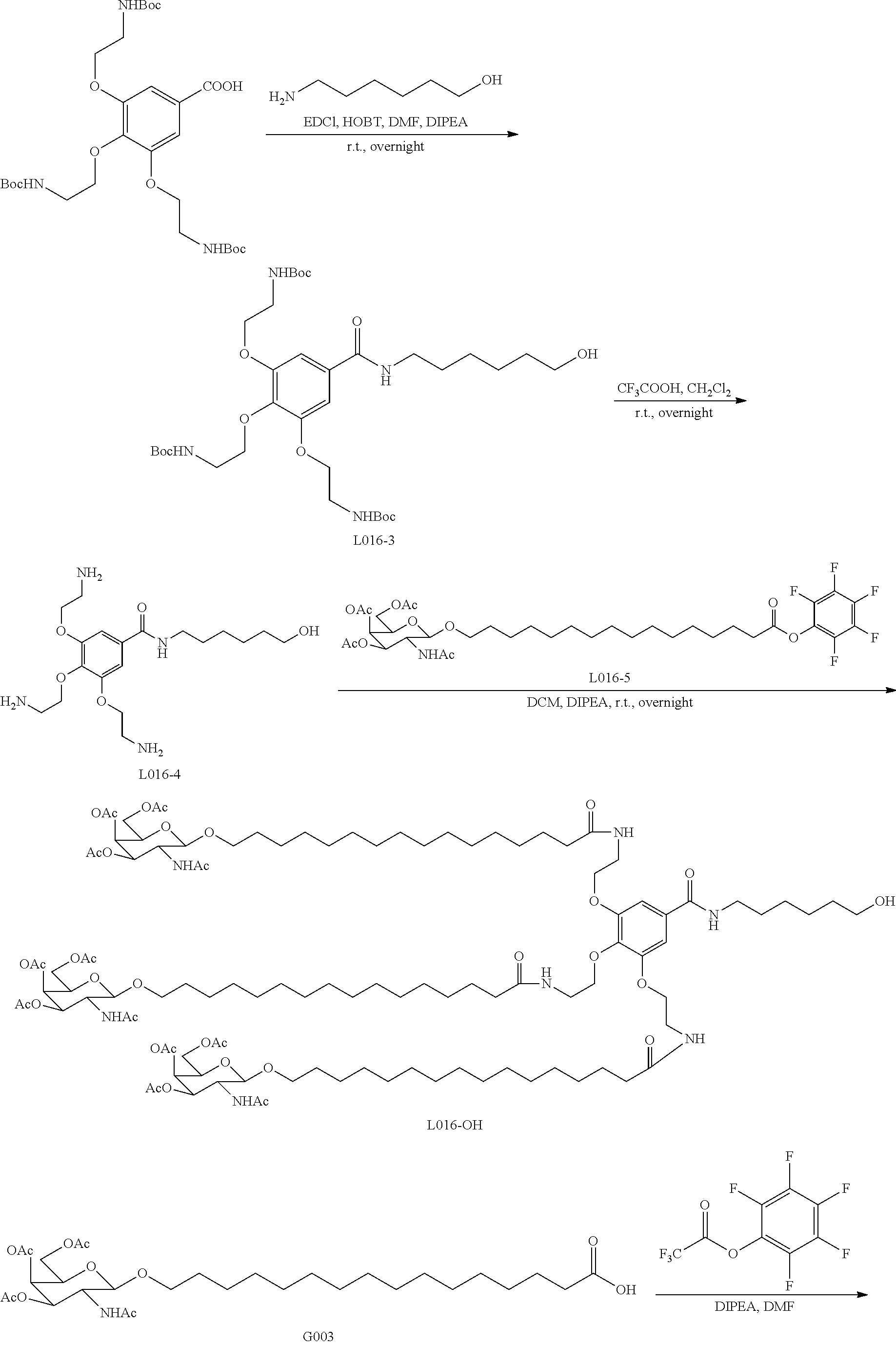

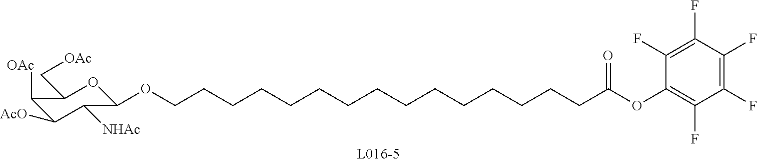 [SMILES: CC(=O)NC1C(OCCCCCCCCCCCCCCCC(=O)Oc2c(F)c(F)c(F)c(F)c2F)OC(COC(C)=O)C(OC(C)=O)C1OC(C)=O]

### Step 1: Synthesis of L016-3

To a solution of 3,4,5-tris(2-((tert-butoxycarbonyl)amino)ethoxy)benzoic acid (2.45 g, 4.1 mmol) in DMF (60 mL) was added EDCI (1.0 g, 5.2 mmol), HOBT (0.70 g, 5.2 mmol) and DIPEA (1.5 mL, 8.6 mmol). The resulting solution was stirred at room temperature for 10 min., then 6-aminohexan-1-ol (0.45 g, 3.8 mmol) was added and stirred for about 4 h. The reaction was quenched with H₂O (40 mL) followed by extraction with ethyl acetate (60 mL×2), and dried over anhydrous Na₂SO₄. Then the residue was purified on a silica gel column to yield L016-3 as a white solid (2.50 g, 93%). MS Calcd: 698.4; Found: 721.5 (M+Na⁺).

### Step 2: Synthesis of L016-4

To a solution of compound L016-3 (0.30 g) in 8 mL dichloromethane was added trifluoroacetic acid 1.5 mL, then stirred at room temperature overnight. Evaporated to give a thick oil without further purification.

### Step 3: Synthesis of L016-5

To a solution of acid G003 (0.92 g, 1.53 mmol) in 30 mL dichloromethane was added DIPEA (3 mL) and pentafluorophenyl trifluoroacetate (1.5 mL) and stirred at room temperature overnight. The reaction was quenched by cold sat. NaHCO₃ and extracted with DCM (30 mL×2), the combined organic layers were washed with H₂O, dried over Na₂SO₄, filtered, and evaporated to give a brown oil (1.5 g). The residue was purified on silica gel column to yield L016-5 as a colorless oil (1.0 g, 83%).

### Step 4: Synthesis of L016-OH

To a solution of compound L016-5 (1.0 g, 1.30 mmol) in 20 mL THF was added DIPEA (2 mL) and compound L016-4 (0.17 g, 0.43 mmol) in 10 mL THF, stirred at room temperature for 16 h. The reaction was quenched with water and extracted with ethyl acetate (30 mL×2), the combined organic layers were dried over Na₂SO₄, filtered and evaporated in vacuo. The residue was purified on silica gel column to yield L016-OH as a white solid (0.96 g, 96%). MS Calcd: 2148.3; Found: 1076.40 [M/2+H]⁺. ¹H NMR (400 MHz, DMSO-d₆): δ 8.50 (s, 1H), 8.1 (m, 2H), 7.90 (m, 3H), 7.20(S, 2H), 5.20(d, 3H), 4.90(m, 3H), 4.50 (d, 3H), 4.34 (t, 1H), 4.04 (m, 12H), 3.80 (m, 5H), 3.70 (m, 3H), 3.65-3.20(m, 12H), 3.0(m, 6H), 2.20 (m, 15H), 2.11 (s, 9H), 2.00 (s, 9H), 1.90 (s, 9H), 1.77 (m, 16H), 1.16-1.49 (m, 87H).

### Step 5: Phosphoramidite Formation and Oligonucleotide-Conjugate Synthesis

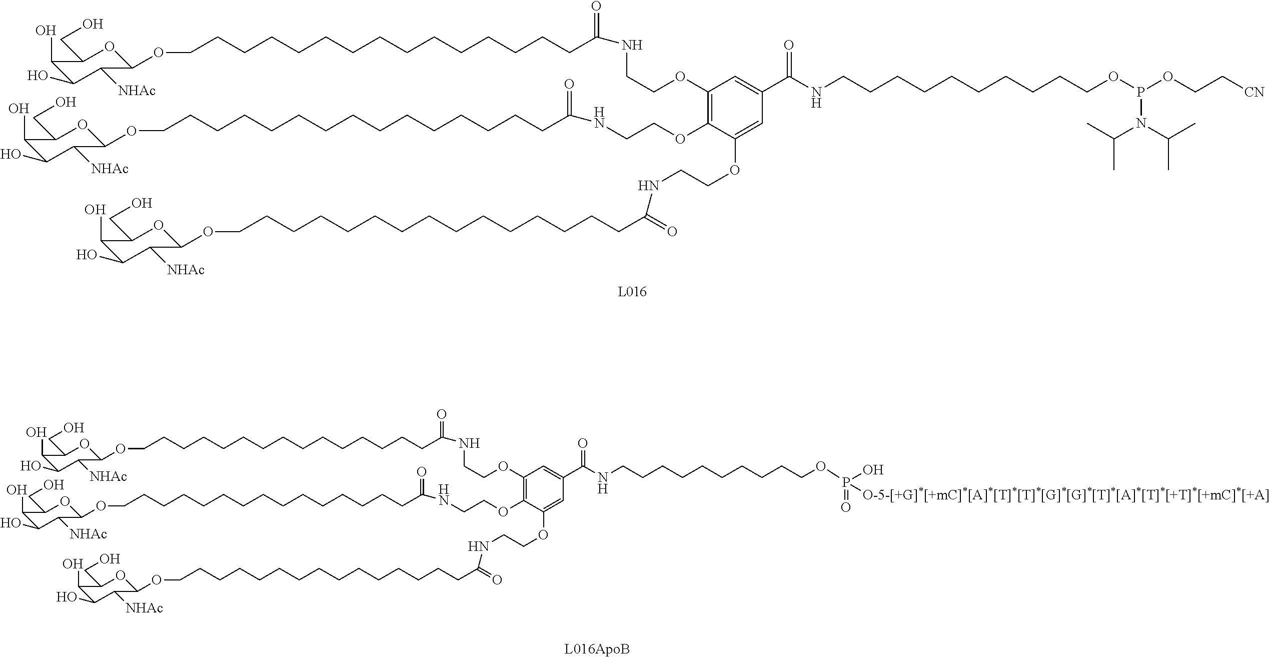

The general method described in examples 2, 3, and 5 was used to synthesize L016 and L016-ApoB conjugates.

L016, MS calculated: 2348.4; Observed: 1197.1 (M/2+Na⁺). ³¹P-NMR (DMSO-d₆), 147.6 ppm.

L016ApoB, MS calculated: 6157.6; found: 6158.3.

### Example 11-2

### Synthesis of L017-OH

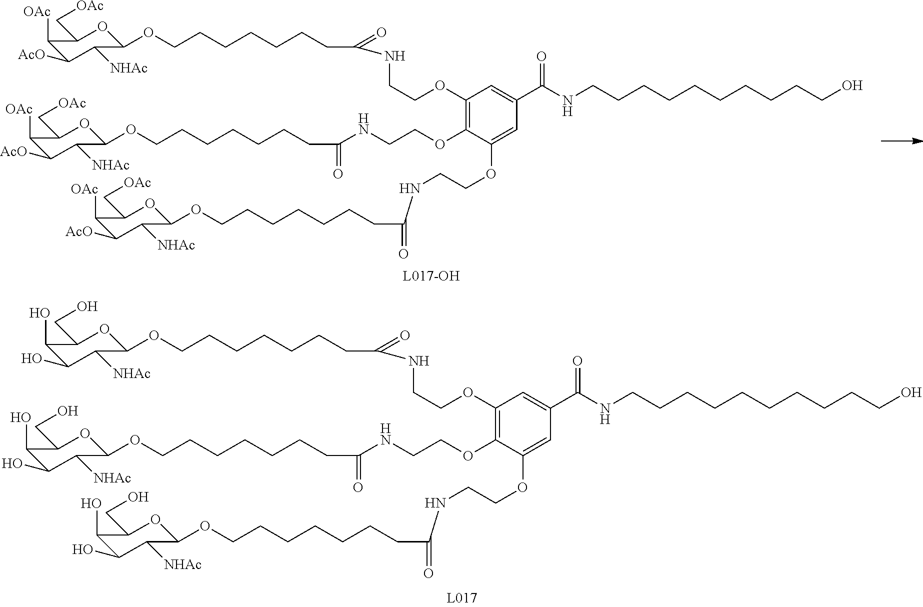 [SMILES: CC(=O)NC1C(OCCCCCCCC(=O)NCCOc2cc(C(=O)NCCCCCCCCCCO)cc(OCCNC(=O)CCCCCCCOC3OC(COC(C)=O)C(OC(C)=O)C(OC(C)=O)C3NC(C)=O)c2OCCNC(=O)CCCCCCCOC2OC(COC(C)=O)C(OC(C)=O)C(OC(C)=O)C2NC(C)=O)OC(COC(C)=O)C(OC(C)=O)C1OC(C)=O ; CC(=O)NC1C(OCCCCCCCC(=O)NCCOc2cc(C(=O)NCCCCCCCCCCO)cc(OCCNC(=O)CCCCCCCOC3OC(CO)C(O)C(O)C3NC(C)=O)c2OCCNC(=O)CCCCCCCOC2OC(CO)C(O)C(O)C2NC(C)=O)OC(CO)C(O)C1O]

L017-OH and L017 were synthesized using a similar method as for L016-OH and L016.

L017-OH, MS Calcd: 1867.9; Found: 935.6.0 [M/2+H]⁺. ¹H NMR (400 MHz, DMSO-d₆): δ 8.40 (m, 1H), 8.1 (m, 2H), 7.90 (m, 4H), 7.20(S, 2H), 5.20(d, 3H), 4.90(m, 3H), 4.50 (d, 3H), 4.34 (t, 1H), 4.04 (m, 12H), 3.90 (m, 5H), 3.70 (m, 3H), 3.60-3.30(m, 10H), 3.20(m, 3H), 2.80(m, 3H), 2.20 (m, 15H), 2.11 (s, 9H), 2.00 (s, 9H), 1.90 (s, 9H), 1.77 (m, 15H), 1.16-1.49 (m, 36H).

L017, ³¹P-NMR (DMSO-d₆), 146.7 ppm.

L017-ApoB conjugate, MS calculated: 5877.3; found: 5877.4.

### Example 11-3

### Synthesis of L031-OH

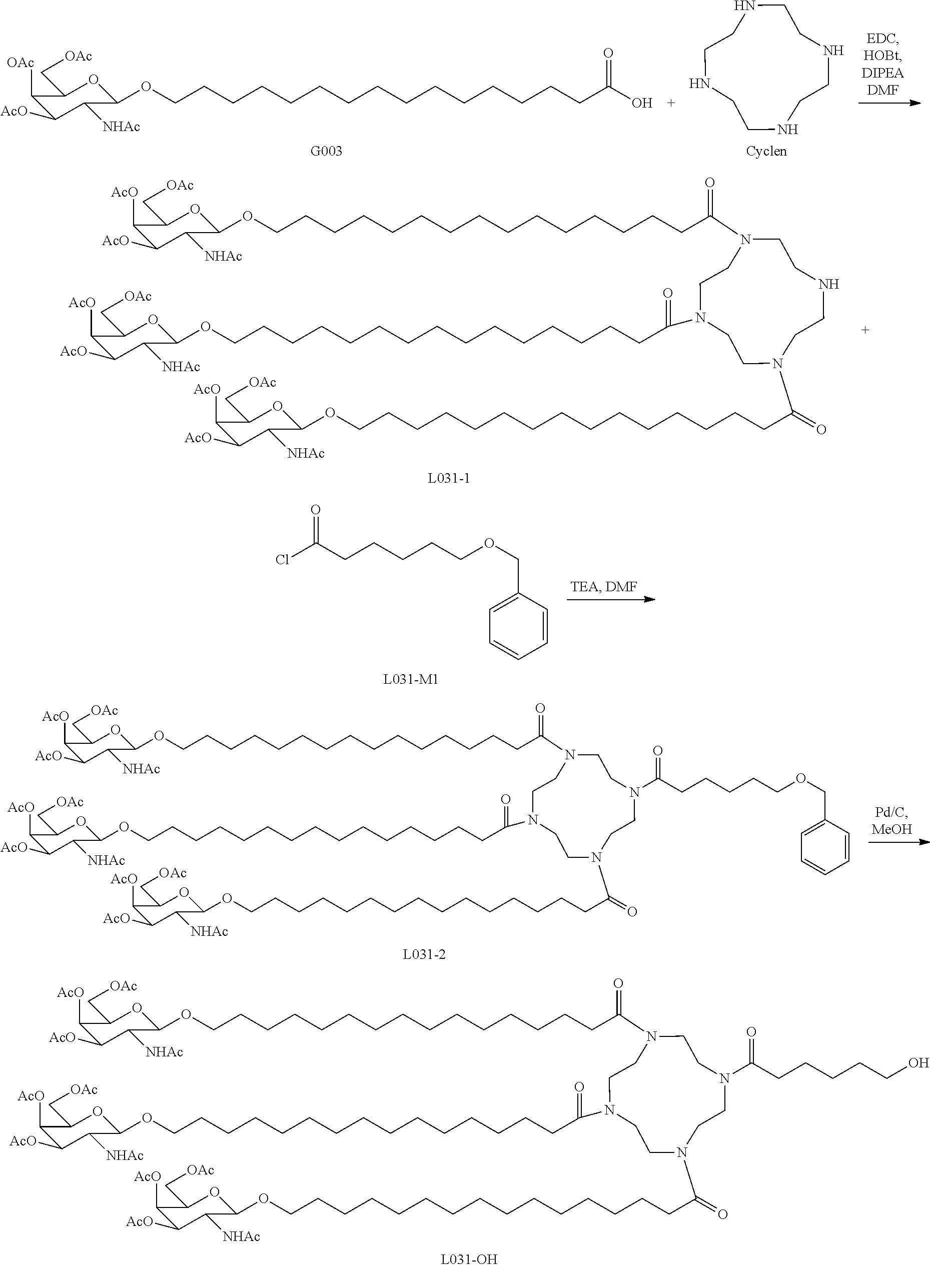

### Step 1: Synthesis of L031-1

To a solution of G003 (1.37 g, 2.3 mmol) in 20 mL of anhydrous DMF, DIPEA (775 mg, 6.0 mmol), EDCI (520 mg, 2.7 mmol) and HOBt (370 mg, 2.7 mmol) were added. The reaction was stirred at room temperature for 0.5 h and cyclen (103 mg, 0.6 mmol) was added. The mixture was stirred at room temperature for more than 24 h after the reaction was completed, then ethyl acetate 100 mL and brine 30 mL were added to dilute the reaction and the mixture was stirred for 10min. The organic phase was separated, the aqueous phase was extracted by ethyl acetate (50 mL×2). The organic phase was combined and dried over anhydrous sodium sulfate. The residue was purified by silica gel column (MeOH/EA=0% to 5%) to provide the compound L031-1 (750 mg, 65.2% yield) as a white solid. MS Calcd: 1754.0; Found: 878.7 (M/2+H⁺).

### Step 2: Synthesis of L031-2

To a solution of benzyl-protected 6-hydroxyl hexanoic acid (130 mg, 0.59 mmol) in 3.0 mL of anhydrous THF, two drops of DMF were added. Oxalyl chloride (123 mg, 0.98 mmol) was added dropwise with stirring. After a reaction time of 2 hours, the mixture was evaporated in vacuo to dryness. 5 mL of anhydrous THF was added and the mixture was evaporated in vacuo to dry. The residue was diluted by 4 mL of DCM and the solution (L031-M1) was directly used. To a solution of L031-1 (750 mg, 0.39 mmol) in 10 mL of anhydrous DCM, DIPEA (504 mg, 3.9 mmol) was added. The mixture was stirred in an ice bath until the temperature dropped below 5° C. With stirring, L031-M1 solution was added dropwise at a temperature of 0-10° C. After addition, the ice bath was removed, and the reaction mixture was stirred for 1 h. When the reaction was completed, ethyl acetate (50 mL) and brine (30 mL) were added and the mixture was stirred for 10 min. The organic phase was separated, and the aqueous phase was extracted by ether acetate (30 mL×2). The organic phase was separated, dried over anhydrous sodium sulfate, and evaporated to a pale yellow oil. The residue was separated by silica gel column (MeOH/EA=0% to 5%) to provide the compound L031-2 (400 mg, 53.3% yield) as a white solid. MS Calcd: 1958.1; Found: 980.8 (M/2+H⁺).

### Step 3: Synthesis of L031-OH

L031-2 (400 mg, 0.19 mmol) was dissolved in 8 mL of methanol and Pd/C (120 mg) was added. The mixture was degassed and charged with argon 3 times. Then the mixture was stirred for 24 h at room temperature. After the reaction was completed, the system was filtered until the solution was clear. The clear solution was evaporated to dry to obtain the compound L031-OH (320 mg, 84.2% yield). MS Calcd: 2036.2; Found: 2038.0 [M+H]⁺. ¹H NMR (400 MHz, DMSO-d₆): δ 7.81 (d, J=9.2 Hz, 3H), 5.22 (d, J=4.4Hz, 3H), 4.97 (dd, J=3.6Hz, 11.2Hz, 3H), 4.48 (d, J=8.4Hz,3H), 4.34 (t, J=4.8Hz, 1H), 4.04 (m, 9H), 3.87 (q, J=8.8Hz, 3H), 3.37-3.50 (m, 24H), 2.24 (br, 8H), 2.11 (s, 9H), 2.00 (s, 3H), 1.90 (s, 3H), 1.77 (s, 3H), 1.16-1.49 (m, 84H).

### Example 11-4

### Synthesis of L032-OH

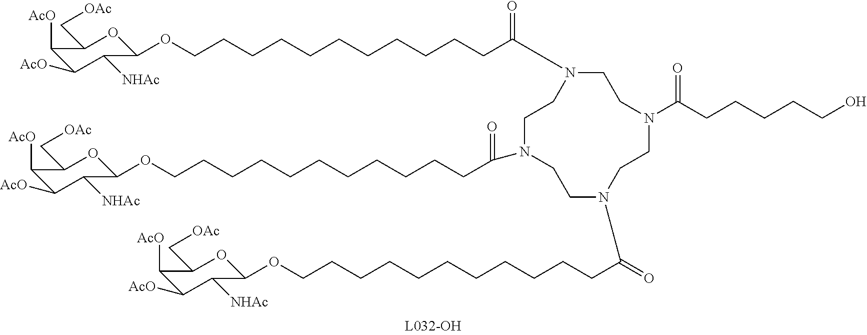 [SMILES: CC(=O)NC1C(OCCCCCCCCCCCC(=O)CN2CCN(C(=O)CCCCCO)CCN(CC(=O)CCCCCCCCCCCOC3OC(COC(C)=O)C(OC(C)=O)C(OC(C)=O)C3NC(C)=O)CCN(C(=O)CCCCCCCCCCCOC3OC(COC(C)=O)C(OC(C)=O)C(OC(C)=O)C3NC(C)=O)CC2)OC(COC(C)=O)C(OC(C)=O)C1OC(C)=O]

L032-OH was synthesized in a similar manner as L031-OH: (440 mg, 78.5% yield). MS Calcd: 1868.1; Found: 1887.0 [M+H₂O+H]⁺. ¹H NMR (400 MHz, DMSO-d₆): δ 7.81 (d, J=9.2 Hz, 3H), 5.22 (d, J=3.6Hz, 3H), 4.97 (dd, J=3.6Hz, 11.2Hz, 3H), 4.49 (d, J=8.0Hz, 3H), 4.34 (t, J=5.2Hz, 1H), 4.03 (m, 9H), 3.87 (q, J=9.2 Hz, 3H), 3.36-3.72 (m, 24H), 2.30 (br, 8H), 2.11 (s, 9H), 2.00 (s, 3H), 1.90 (s, 3H), 1.77 (s, 3H), 1.20-1.49 (m, 60H).

### Effect Example

### 1. Screening the Efficacy of GalNAc Clusters Through an ApoB Reduction Assay.

The oligonucleotide-GalNAc conjugates for the studies were prepared as described in Examples 4 and 5 and formulated in PBS for studies. Mice were grouped based on BW on day −4. The study was performed for up to 30 days to evaluate the durability of target knockdown achieved with each conjugate. Mice were dosed once on day 0 at two dose levels (high, 60 nmoles/kg and low, 20 nmoles/kg) and bled on days 3, 10, and 17 to monitor plasma Apo B protein levels. The study was terminated on the last study observation day, or humane endpoint whichever came first. Blood (˜50 uL/mouse/timepoint) via tail or retro orbital bleeding was collected into EDTA-coated tubes. Blood samples were centrifuged for 10 minutes at 1,000-2,000×g in a refrigerated centrifuge. Following centrifugation, the resulting supernatant (plasma) was immediately transferred into a clean labeled polypropylene tube and stored at −80° C. until use.

Plasma ApoB levels were determined using a commercial ELISA kit (AbCam #ab230932). The assay was performed according to the manufacturer's instructions. Plasma samples were tested at 10000-fold dilutions in duplicate. ApoB results were reported either as ug/mL or normalized to initial Apo B levels determined prior to dosing of oligonucleotide-GalNAc conjugates. The comparison between compounds were used to elucidate structure-activity relationships (SAR) and the comparison to tri-antennary positive control was used to select active GalNAc moieties.

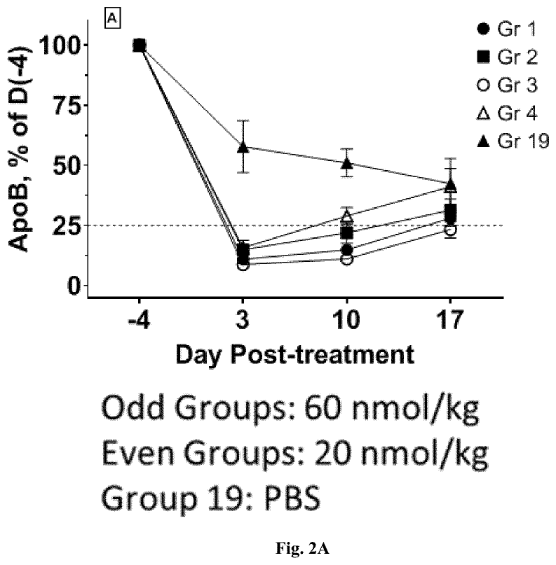

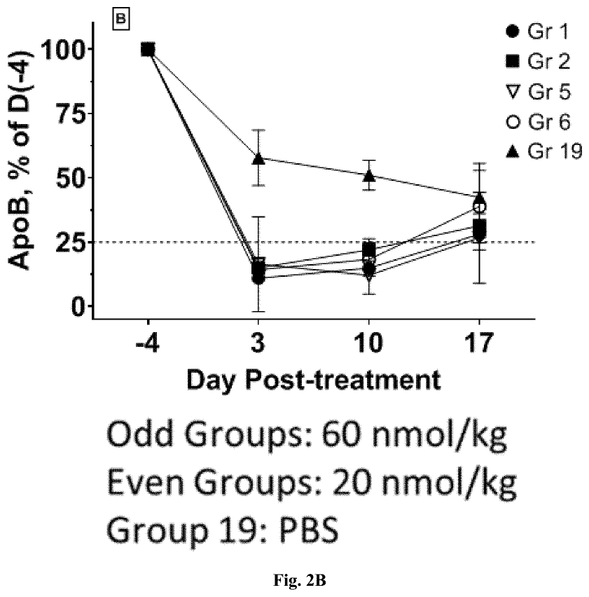

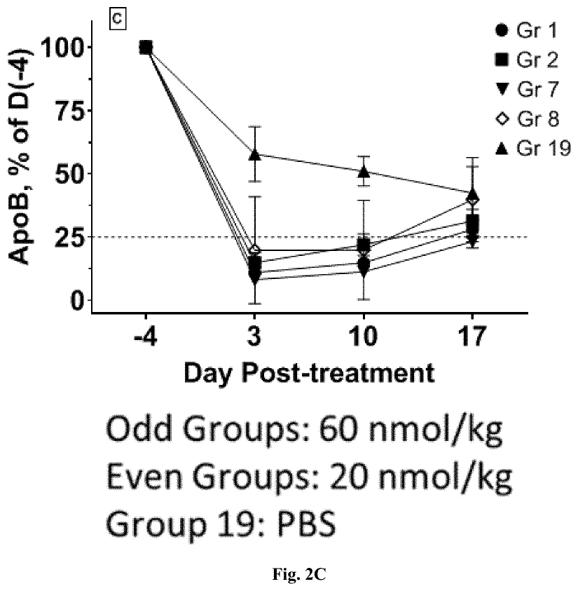

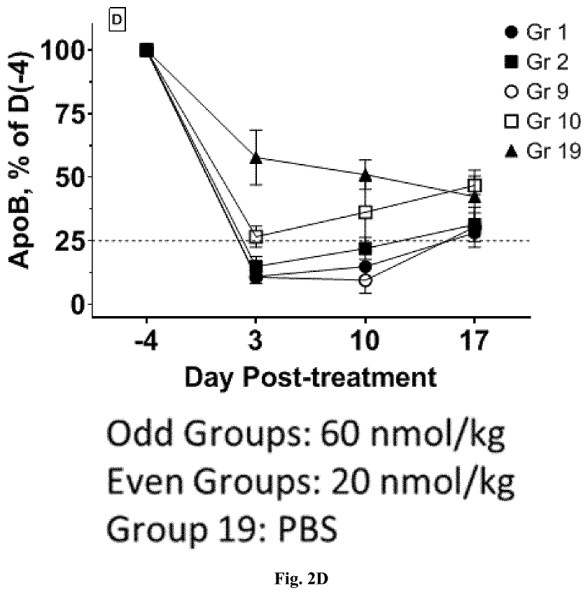

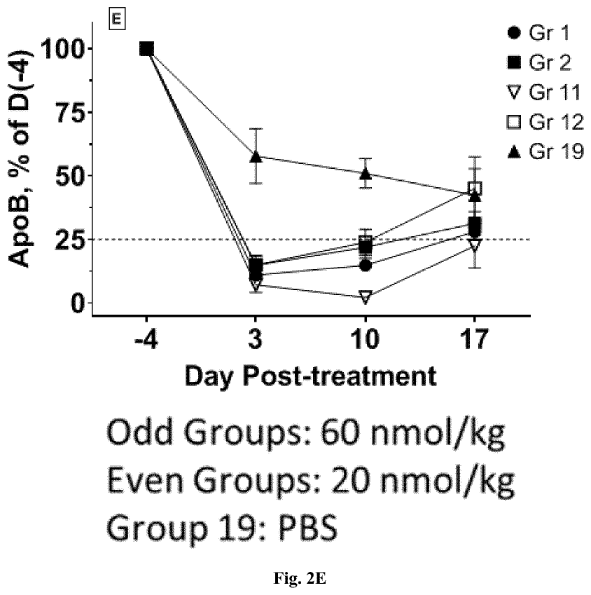

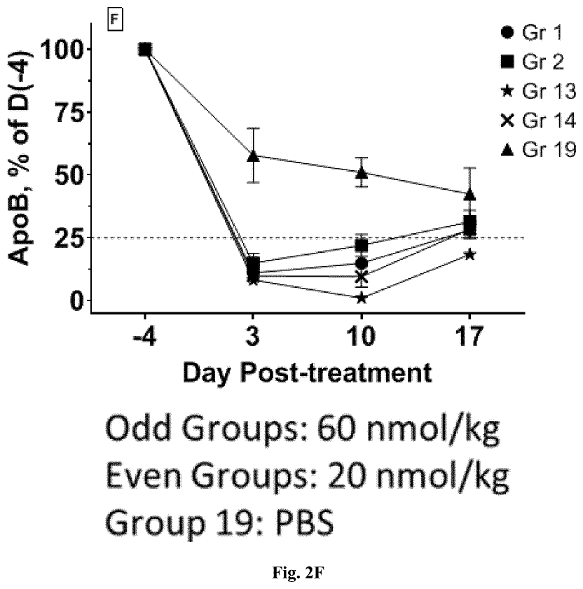

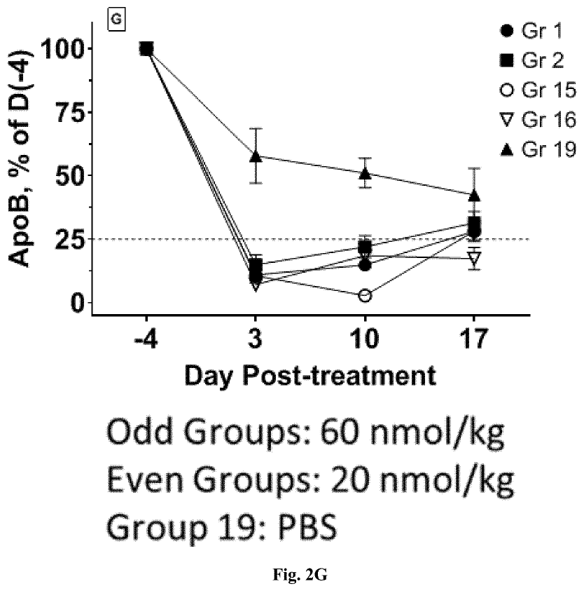

What is unexpected is that the majority of GalNAc clusters (include B006-group 3/4, B007-group 5/6, B008-group 7/8, B009-group 9/10, B011-group 11/12, B013-group 13/14, and B015-group 15/16) synthesized with repeat addition of monomers have shown similar or better durability of Apo B knockdown compared with positive control B005. Some of the clusters represented in group 11/12 and 13/14 (GalNAc clusters B011 and B013) in fact showed greater efficiency from day 10 to day 17 (see FIG. 2A-2G).

### Other Embodiments

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary, to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A conjugate, which comprises a structure represented by formula (I) below: 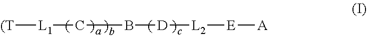
wherein:
T is a liver cell-targeting ligand;
L₁ and L₂ are independently a tether group;
C is a linker group;
B is a branching group;
D is linker group
E is ester group;
A is antisense sequence or passenger strand of siRNA;
a is 0 or 1;
b is an integer between 1-5; and
c is 1 or 2.

2. The conjugate of claim 1, wherein T is selected from glucose, mannose, galactose, N-acetyl-galactosamine, fucose, glucosamine, N-acetyl-mannosamine, lactose, maltose, or folate.

3. The conjugate of claim 1, wherein L₁ and L₂ are independently selected from C₁-C₂₀ alkylene, amide, or (C₁-C₂₀) alkylene-amide-(C₁-C₂₀) alkylene.

4. The conjugate of claim 3, wherein L₁ and L₂ are independently selected from —(CH₂)ₙ—, —(CH₂)ₘ—CONH—(CH₂)ₘ—, or —(CH₂)ₘ—NHCO—(CH₂)ₘ—, wherein m is an integer between 1-9, and n is an integer between 5-20.

5. The conjugate of claim 1, wherein C is selected from C₁-C₂₀ alkylene, amide, carbonyl, amide-(C₁-C₂₀) alkylene, or carbonyl-heterocyclic ring-phosphate-(C₁-C₁₀) alkylene.

6. The conjugate of claim 5, wherein C is selected from: 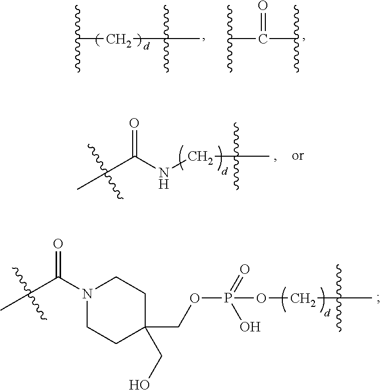
wherein d is an integer between 0-5.

7. The conjugate of claim 1, wherein B is a di-antennary branching group, tri-antennary branching group, tetra-antennary branching group, penta-antennary branching group, or hexa-antennary branching group.

8. The conjugate of claim 7, wherein B is selected from: 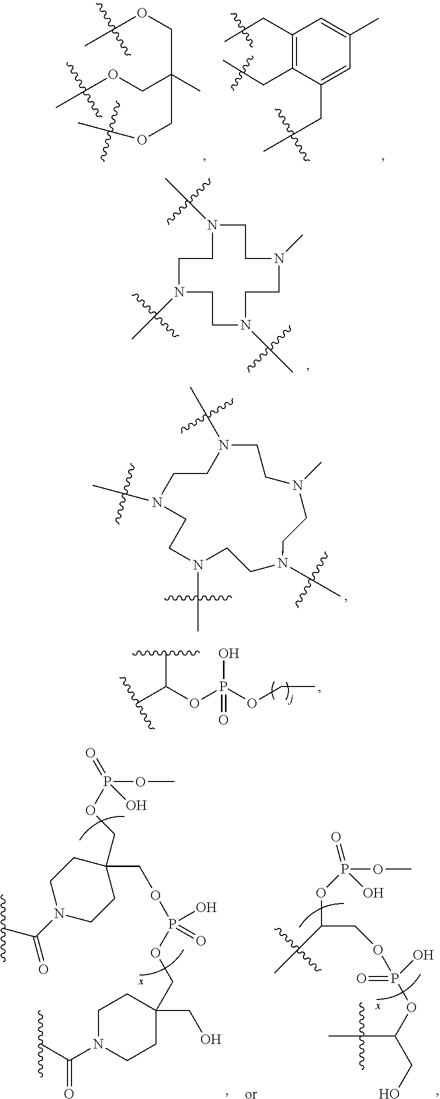
wherein x is an integer between 1-5; and
j is an integer between 0-5.

9. The conjugate of claim 1, wherein D is selected from C₁-C₂₀ alkylene, amide, carbonyl, or (C₁-C₂₀) alkylene-amide-(C₁-C₂₀) alkylene.

10. The conjugate of claim 9, wherein D is selected from —(CH₂)ₖ—, 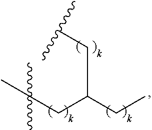
—(C═O)—, —CONH—, or —NHCO—; wherein k is an integer between 0-5.

11. The conjugate of claim 1, wherein E is

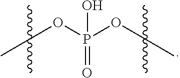 [SMILES: *OP(=O)(O)O*]

12. The conjugate of claim 1, wherein:
T is selected from glucose, mannose, galactose, N-acetyl-galactosamine, fucose, glucosamine, N-acetyl-mannosamine, lactose, maltose, or folate;
L₁ and L₂ are independently selected from —(CH₂)ₙ—, —(CH₂)ₘ—CONH—(CH₂)ₘ—, or —(CH₂)ₘ—NHCO—(CH₂)ₘ—; wherein:
m is an integer between 1-9;
n is an integer between 5-20;
C is selected from: 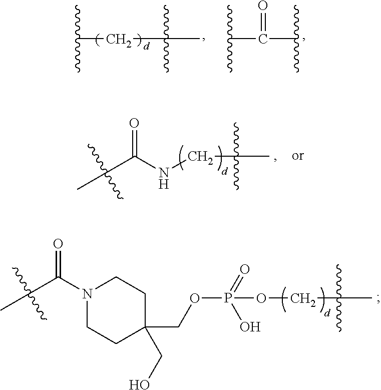
or
wherein d is an integer between 0-5;
B is selected from: 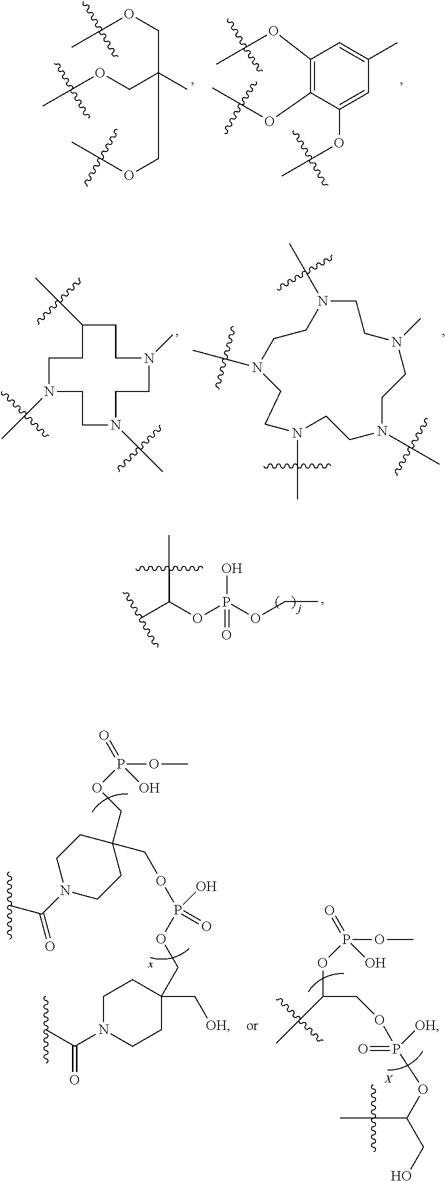
wherein x is an integer between 1-5;
j is an integer between 0-5;
D is selected from —(CH₂)ₖ—, 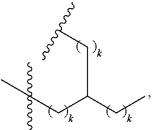
—(C═O)—, —CONH—, or —NHCO—;
wherein k is an integer between 0-5; and
E is

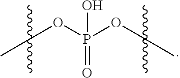 [SMILES: *OP(=O)(O)O*]

13. The conjugate of claim 12, wherein:
C is selected from: 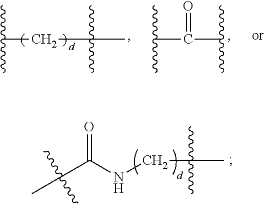
wherein d is an integer between 0-5;
B is selected from: 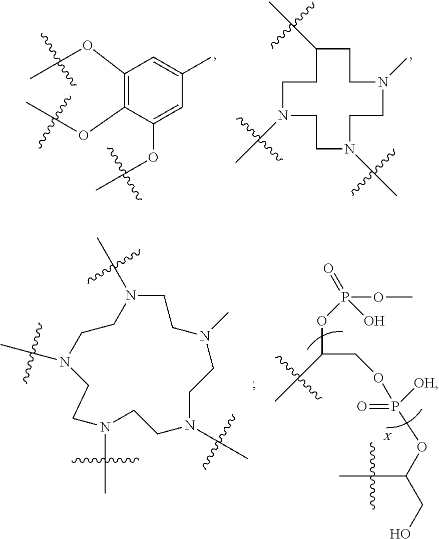
wherein x is an integer between 1-5; and
D is selected from —(CH₂)ₖ—, —(C═O)—, —CONH—, or —NHCO—;
wherein k is an integer between 0-5.

14. The conjugate of claim 12,
wherein:
C is selected from: 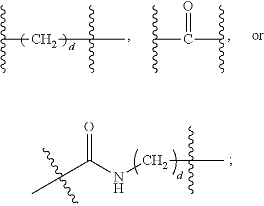
, or
wherein d is an integer between 0-5;
B is: 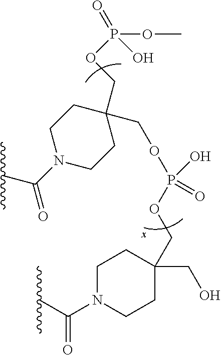
wherein x is an integer between 1-5; and
D is selected from —(CH₂)ₖ—, —(C═O)—, —CONH—, or —NHCO—;
wherein k is an integer between 0-5.

15. The conjugate of claim 12,
wherein:
C is 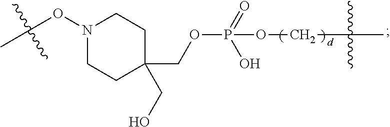
wherein d is an integer between 0-5; and
B is selected from: 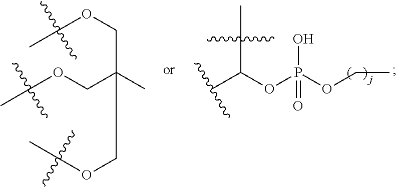
or
wherein j is an integer between 0-5.

16. The conjugate of claim 12, wherein:
C is: 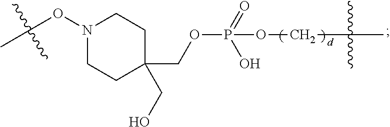
wherein d is an integer between 0-5; and
B is: 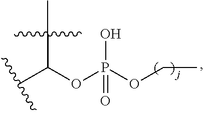
wherein j is an integer between 0-5.

17. The conjugate of claim 1, which comprises a structure represented below: 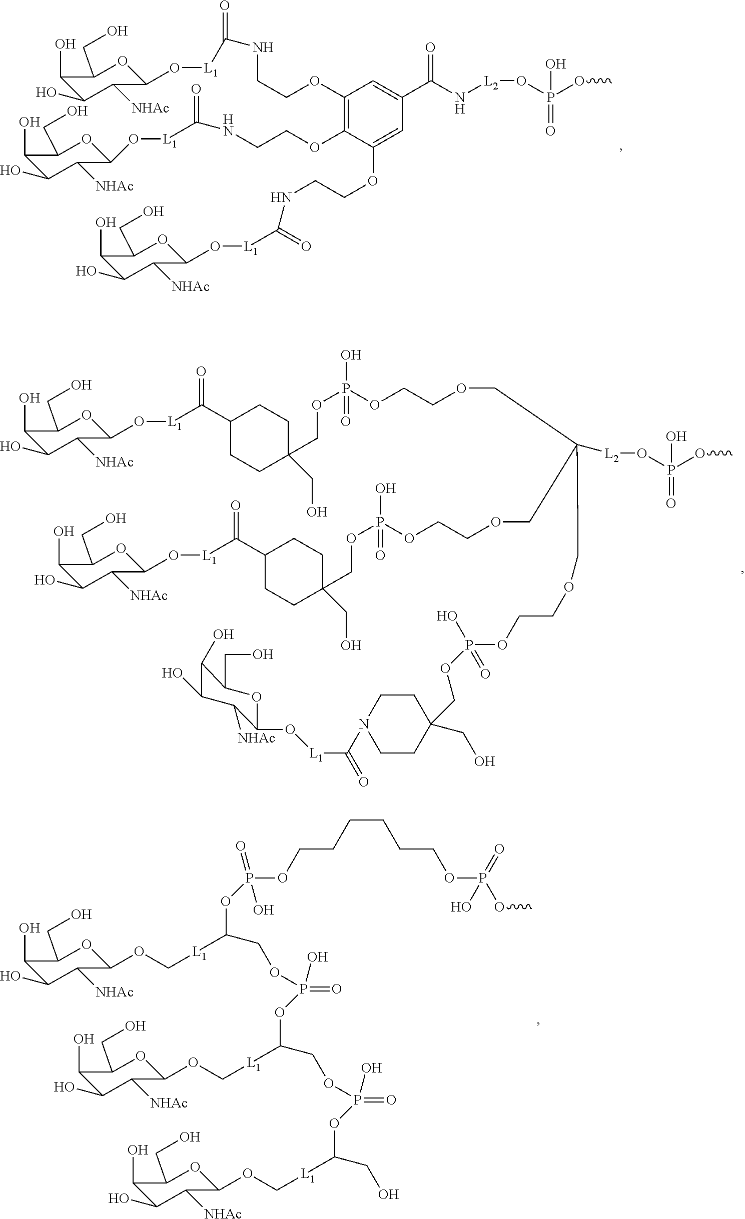 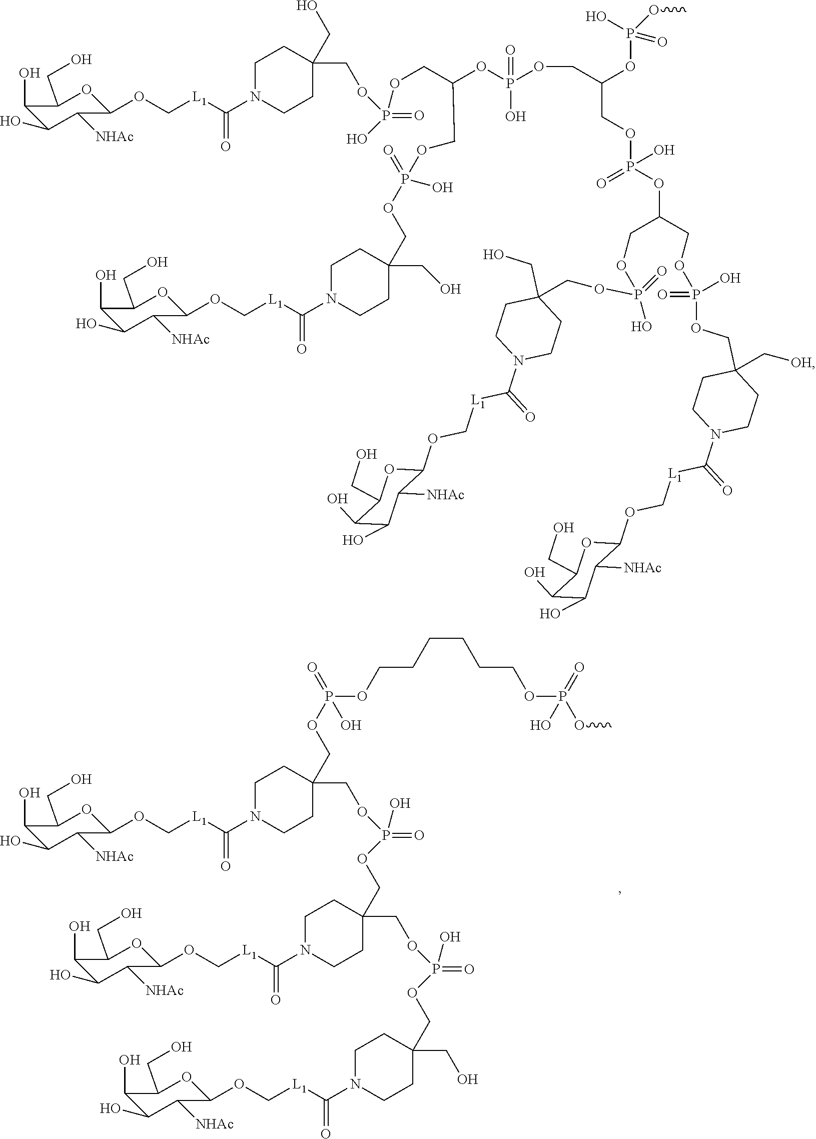 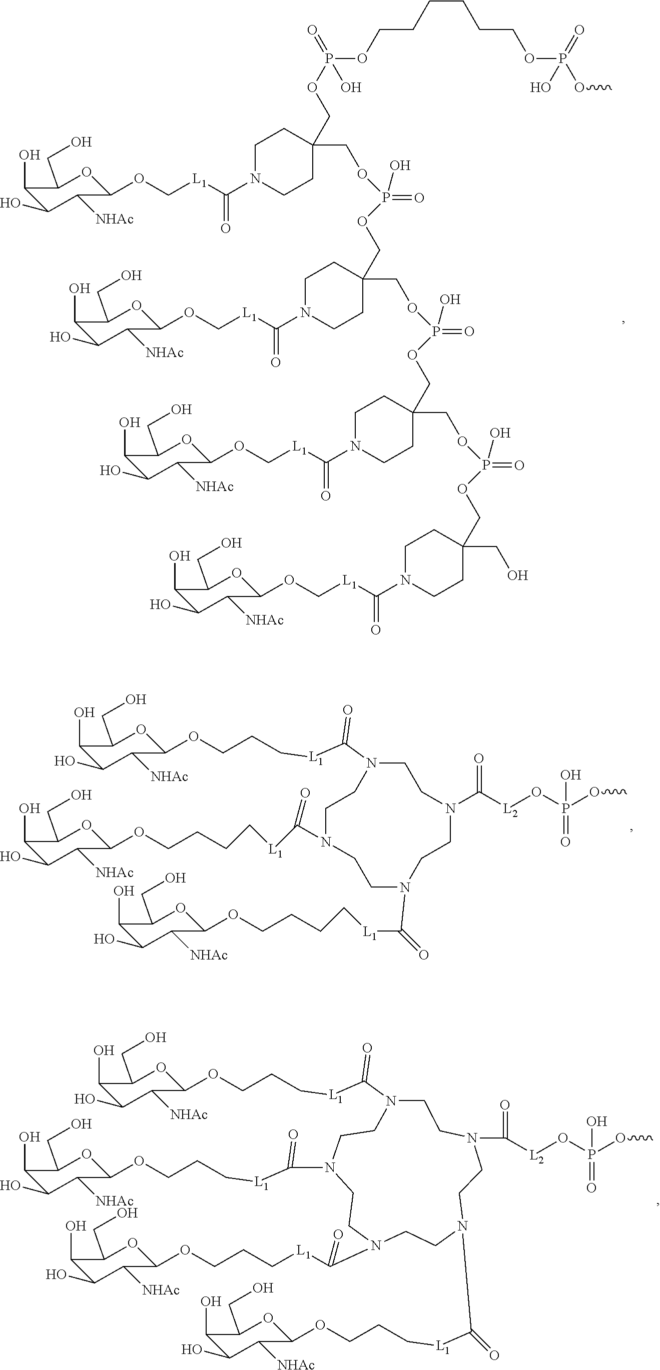
wherein L₁ and L₂ are independently selected from C₁-C₂₀ alkylene, amide, or (C₁-C₂₀) alkylene-amide-(C₁-C₂₀) alkylene.

18. A pharmaceutical composition comprising a conjugate of claim 1 and one or more pharmaceutically acceptable carriers or diluents.

19. A method of targeting the liver to treat a disease comprising administering to a mammal in need thereof a therapeutically effective amount of a pharmaceutical composition of claim 18.

20. The method of claim 19, wherein the disease is an RNA-dependent viral infection.


SEQUENCE LISTING (273 sequences)

SEQ ID NO: 1 - DNA, 16 bp, Artificial Sequence
  Antisense Oligonucleotide
gagagaagtccaccac

SEQ ID NO: 2 - DNA, 16 bp, Artificial Sequence
  Antisense Oligonucleotide
tgagagaagtccacca

SEQ ID NO: 3 - DNA, 16 bp, Artificial Sequence
  Antisense Oligonucleotide
gaggcatagcagcagg

SEQ ID NO: 4 - DNA, 16 bp, Artificial Sequence
  Antisense Oligonucleotide
tgaggcatagcagcag

SEQ ID NO: 5 - DNA, 16 bp, Artificial Sequence
  Antisense Oligonucleotide
gatgaggcatagcagc

SEQ ID NO: 6 - DNA, 16 bp, Artificial Sequence
  Antisense Oligonucleotide
gatgggatgggaatac

SEQ ID NO: 7 - DNA, 16 bp, Artificial Sequence
  Antisense Oligonucleotide
ggcccactcccatagg

SEQ ID NO: 8 - DNA, 16 bp, Artificial Sequence
  Antisense Oligonucleotide
aggcccactcccatag

SEQ ID NO: 9 - DNA, 16 bp, Artificial Sequence
  Antisense Oligonucleotide
ctgaggcccactccca

SEQ ID NO: 10 - DNA, 20 bp, Artificial Sequence
  Antisense Oligonucleotide
gcagaggtgaagcgaagtgc

SEQ ID NO: 11 - DNA, 17 bp, Artificial Sequence
  Antisense Oligonucleotide
ccacgagtctagactct

SEQ ID NO: 12 - DNA, 17 bp, Artificial Sequence
  Antisense Oligonucleotide
gtccaccacgagtctag

SEQ ID NO: 13 - DNA, 17 bp, Artificial Sequence
  Antisense Oligonucleotide
agtccaccacgagtcta

SEQ ID NO: 14 - DNA, 17 bp, Artificial Sequence
  Antisense Oligonucleotide
aagtccaccacgagtct

SEQ ID NO: 15 - DNA, 17 bp, Artificial Sequence
  Antisense Oligonucleotide
gaagtccaccacgagtc

SEQ ID NO: 16 - DNA, 17 bp, Artificial Sequence
  Antisense Oligonucleotide
agaagtccaccacgagt

SEQ ID NO: 17 - DNA, 17 bp, Artificial Sequence
  Antisense Oligonucleotide
gagaagtccaccacgag

SEQ ID NO: 18 - DNA, 17 bp, Artificial Sequence
  Antisense Oligonucleotide
agagaagtccaccacga

SEQ ID NO: 19 - DNA, 17 bp, Artificial Sequence
  Antisense Oligonucleotide
gagagaagtccaccacg

SEQ ID NO: 20 - DNA, 17 bp, Artificial Sequence
  Antisense Oligonucleotide
tgagagaagtccaccac

SEQ ID NO: 21 - DNA, 17 bp, Artificial Sequence
  Antisense Oligonucleotide
tgataaaacgccgcaga

SEQ ID NO: 22 - DNA, 17 bp, Artificial Sequence
  Antisense Oligonucleotide
atgataaaacgccgcag

SEQ ID NO: 23 - DNA, 17 bp, Artificial Sequence
  Antisense Oligonucleotide
ggcatagcagcaggatg

SEQ ID NO: 24 - DNA, 17 bp, Artificial Sequence
  Antisense Oligonucleotide
aggcatagcagcaggat

SEQ ID NO: 25 - DNA, 17 bp, Artificial Sequence
  Antisense Oligonucleotide
gaggcatagcagcagga

SEQ ID NO: 26 - DNA, 20 bp, Artificial Sequence
  Antisense Oligonucleotide
agatgaggcatagcagcagg

SEQ ID NO: 27 - DNA, 20 bp, Artificial Sequence
  Antisense Oligonucleotide
aagatgaggcatagcagcag

SEQ ID NO: 28 - DNA, 17 bp, Artificial Sequence
  Antisense Oligonucleotide
atgaggcatagcagcag

SEQ ID NO: 29 - DNA, 20 bp, Artificial Sequence
  Antisense Oligonucleotide
gaagatgaggcatagcagca

SEQ ID NO: 30 - DNA, 17 bp, Artificial Sequence
  Antisense Oligonucleotide
gatgaggcatagcagca

SEQ ID NO: 31 - DNA, 20 bp, Artificial Sequence
  Antisense Oligonucleotide
agaagatgaggcatagcagc

SEQ ID NO: 32 - DNA, 17 bp, Artificial Sequence
  Antisense Oligonucleotide
agatgaggcatagcagc

SEQ ID NO: 33 - DNA, 20 bp, Artificial Sequence
  Antisense Oligonucleotide
aagaagatgaggcatagcag

SEQ ID NO: 34 - DNA, 17 bp, Artificial Sequence
  Antisense Oligonucleotide
aagatgaggcatagcag

SEQ ID NO: 35 - DNA, 17 bp, Artificial Sequence
  Antisense Oligonucleotide
agaagatgaggcatagc

SEQ ID NO: 36 - DNA, 17 bp, Artificial Sequence
  Antisense Oligonucleotide
aagaagatgaggcatag

SEQ ID NO: 37 - DNA, 17 bp, Artificial Sequence
  Antisense Oligonucleotide
acgggcaacataccttg

SEQ ID NO: 38 - DNA, 20 bp, Artificial Sequence
  Antisense Oligonucleotide
ctgaggcccactcccatagg

SEQ ID NO: 39 - DNA, 17 bp, Artificial Sequence
  Antisense Oligonucleotide
aggcccactcccatagg

SEQ ID NO: 40 - DNA, 17 bp, Artificial Sequence
  Antisense Oligonucleotide
gaggcccactcccatag

SEQ ID NO: 41 - DNA, 17 bp, Artificial Sequence
  Antisense Oligonucleotide
tgaggcccactcccata

SEQ ID NO: 42 - DNA, 17 bp, Artificial Sequence
  Antisense Oligonucleotide
ctgaggcccactcccat

SEQ ID NO: 43 - DNA, 20 bp, Artificial Sequence
  Antisense Oligonucleotide
cgaaccactgaacaaatggc

SEQ ID NO: 44 - DNA, 17 bp, Artificial Sequence
  Antisense Oligonucleotide
accactgaacaaatggc

SEQ ID NO: 45 - DNA, 17 bp, Artificial Sequence
  Antisense Oligonucleotide
aaccactgaacaaatgg

SEQ ID NO: 46 - DNA, 17 bp, Artificial Sequence
  Antisense Oligonucleotide
gaaccactgaacaaatg

SEQ ID NO: 47 - DNA, 17 bp, Artificial Sequence
  Antisense Oligonucleotide
cgaaccactgaacaaat

SEQ ID NO: 48 - DNA, 17 bp, Artificial Sequence
  Antisense Oligonucleotide
accacatcatccatata

SEQ ID NO: 49 - DNA, 17 bp, Artificial Sequence
  Antisense Oligonucleotide
tcagcaaacacttggca

SEQ ID NO: 50 - DNA, 20 bp, Artificial Sequence
  Antisense Oligonucleotide
aatttatgcctacagcctcc

SEQ ID NO: 51 - DNA, 17 bp, Artificial Sequence
  Antisense Oligonucleotide
ttatgcctacagcctcc

SEQ ID NO: 52 - DNA, 20 bp, Artificial Sequence
  Antisense Oligonucleotide
caatttatgcctacagcctc

SEQ ID NO: 53 - DNA, 17 bp, Artificial Sequence
  Antisense Oligonucleotide
tttatgcctacagcctc

SEQ ID NO: 54 - DNA, 20 bp, Artificial Sequence
  Antisense Oligonucleotide
ccaatttatgcctacagcct

SEQ ID NO: 55 - DNA, 17 bp, Artificial Sequence
  Antisense Oligonucleotide
atttatgcctacagcct

SEQ ID NO: 56 - DNA, 20 bp, Artificial Sequence
  Antisense Oligonucleotide
accaatttatgcctacagcc

SEQ ID NO: 57 - DNA, 17 bp, Artificial Sequence
  Antisense Oligonucleotide
aatttatgcctacagcc

SEQ ID NO: 58 - DNA, 17 bp, Artificial Sequence
  Antisense Oligonucleotide
caatttatgcctacagc

SEQ ID NO: 59 - DNA, 17 bp, Artificial Sequence
  Antisense Oligonucleotide
ccaatttatgcctacag

SEQ ID NO: 60 - DNA, 17 bp, Artificial Sequence
  Antisense Oligonucleotide
accaatttatgcctaca

SEQ ID NO: 61 - DNA, 17 bp, Artificial Sequence
  Antisense Oligonucleotide
aggcagaggtgaaaaag

SEQ ID NO: 62 - DNA, 17 bp, Artificial Sequence
  Antisense Oligonucleotide
taggcagaggtgaaaaa

SEQ ID NO: 63 - DNA, 20 bp, Artificial Sequence
  Antisense Oligonucleotide
gcacagcttggaggcttgaa

SEQ ID NO: 64 - DNA, 17 bp, Artificial Sequence
  Antisense Oligonucleotide
cagcttggaggcttgaa

SEQ ID NO: 65 - DNA, 20 bp, Artificial Sequence
  Antisense Oligonucleotide
ggcacagcttggaggcttga

SEQ ID NO: 66 - DNA, 17 bp, Artificial Sequence
  Antisense Oligonucleotide
acagcttggaggcttga

SEQ ID NO: 67 - DNA, 20 bp, Artificial Sequence
  Antisense Oligonucleotide
aggcacagcttggaggcttg

SEQ ID NO: 68 - DNA, 17 bp, Artificial Sequence
  Antisense Oligonucleotide
cacagcttggaggcttg

SEQ ID NO: 69 - DNA, 20 bp, Artificial Sequence
  Antisense Oligonucleotide
aaggcacagcttggaggctt

SEQ ID NO: 70 - DNA, 17 bp, Artificial Sequence
  Antisense Oligonucleotide
gcacagcttggaggctt

SEQ ID NO: 71 - DNA, 20 bp, Artificial Sequence
  Antisense Oligonucleotide
caaggcacagcttggaggct

SEQ ID NO: 72 - DNA, 17 bp, Artificial Sequence
  Antisense Oligonucleotide
ggcacagcttggaggct

SEQ ID NO: 73 - DNA, 20 bp, Artificial Sequence
  Antisense Oligonucleotide
ccaaggcacagcttggaggc

SEQ ID NO: 74 - DNA, 17 bp, Artificial Sequence
  Antisense Oligonucleotide
aggcacagcttggaggc

SEQ ID NO: 75 - DNA, 17 bp, Artificial Sequence
  Antisense Oligonucleotide
aaggcacagcttggagg

SEQ ID NO: 76 - DNA, 17 bp, Artificial Sequence
  Antisense Oligonucleotide
caaggcacagcttggag

SEQ ID NO: 77 - DNA, 17 bp, Artificial Sequence
  Antisense Oligonucleotide
ccaaggcacagcttgga

SEQ ID NO: 78 - DNA, 17 bp, Artificial Sequence
  Antisense Oligonucleotide
gctccaaattctttata

SEQ ID NO: 79 - DNA, 20 bp, Artificial Sequence
  Antisense Oligonucleotide
tctgcgaggcgagggagttc

SEQ ID NO: 80 - DNA, 17 bp, Artificial Sequence
  Antisense Oligonucleotide
gcgaggcgagggagttc

SEQ ID NO: 81 - DNA, 17 bp, Artificial Sequence
  Antisense Oligonucleotide
tgcgaggcgagggagtt

SEQ ID NO: 82 - DNA, 17 bp, Artificial Sequence
  Antisense Oligonucleotide
ctgcgaggcgagggagt

SEQ ID NO: 83 - DNA, 17 bp, Artificial Sequence
  Antisense Oligonucleotide
tctgcgaggcgagggag

SEQ ID NO: 84 - DNA, 17 bp, Artificial Sequence
  Antisense Oligonucleotide
ttcccaagaatatggtg

SEQ ID NO: 85 - DNA, 17 bp, Artificial Sequence
  Antisense Oligonucleotide
gttcccaagaatatggt

SEQ ID NO: 86 - DNA, 17 bp, Artificial Sequence
  Antisense Oligonucleotide
tgttcccaagaatatgg

SEQ ID NO: 87 - RNA, 19 nt, Artificial Sequence
  Synthetic
ucgugguggacuucucuca

SEQ ID NO: 88 - RNA, 19 nt, Artificial Sequence
  Synthetic
ugagagaaguccaccacga

SEQ ID NO: 89 - RNA, 19 nt, Artificial Sequence
  Synthetic
gugguggacuucucucaau

SEQ ID NO: 90 - RNA, 19 nt, Artificial Sequence
  Synthetic
auugagagaaguccaccac

SEQ ID NO: 91 - RNA, 19 nt, Artificial Sequence
  Synthetic
gccgauccauacugcggaa

SEQ ID NO: 92 - RNA, 19 nt, Artificial Sequence
  Synthetic
uuccgcaguauggaucggc

SEQ ID NO: 93 - RNA, 19 nt, Artificial Sequence
  Synthetic
ccgauccauacugcggaac

SEQ ID NO: 94 - RNA, 19 nt, Artificial Sequence
  Synthetic
guuccgcaguauggaucgg

SEQ ID NO: 95 - RNA, 19 nt, Artificial Sequence
  Synthetic
cauccugcugcuaugccuc

SEQ ID NO: 96 - RNA, 19 nt, Artificial Sequence
  Synthetic
gaggcauagcagcaggaug

SEQ ID NO: 97 - RNA, 19 nt, Artificial Sequence
  Synthetic
ugcugcuaugccucaucuu

SEQ ID NO: 98 - RNA, 19 nt, Artificial Sequence
  Synthetic
aagaugaggcauagcagca

SEQ ID NO: 99 - RNA, 19 nt, Artificial Sequence
  Synthetic
gguggacuucucucaauuu

SEQ ID NO: 100 - RNA, 19 nt, Artificial Sequence
  Synthetic
aaauugagagaaguccacc

SEQ ID NO: 101 - RNA, 19 nt, Artificial Sequence
  Synthetic
ugguggacuucucucaauu

SEQ ID NO: 102 - RNA, 19 nt, Artificial Sequence
  Synthetic
aauugagagaaguccacca

SEQ ID NO: 103 - RNA, 19 nt, Artificial Sequence
  Synthetic
uagacucgugguggacuuc

SEQ ID NO: 104 - RNA, 19 nt, Artificial Sequence
  Synthetic
gaaguccaccacgagucua

SEQ ID NO: 105 - RNA, 19 nt, Artificial Sequence
  Synthetic
uccucugccgauccauacu

SEQ ID NO: 106 - RNA, 19 nt, Artificial Sequence
  Synthetic
aguauggaucggcagagga

SEQ ID NO: 107 - RNA, 19 nt, Artificial Sequence
  Synthetic
ugccgauccauacugcgga

SEQ ID NO: 108 - RNA, 19 nt, Artificial Sequence
  Synthetic
uccgcaguauggaucggca

SEQ ID NO: 109 - RNA, 19 nt, Artificial Sequence
  Synthetic
uggaugugucugcggcguu

SEQ ID NO: 110 - RNA, 19 nt, Artificial Sequence
  Synthetic
aacgccgcagacacaucca

SEQ ID NO: 111 - RNA, 19 nt, Artificial Sequence
  Synthetic
cgauccauacugcggaacu

SEQ ID NO: 112 - RNA, 19 nt, Artificial Sequence
  Synthetic
aguuccgcaguauggaucg

SEQ ID NO: 113 - RNA, 19 nt, Artificial Sequence
  Synthetic
cgcaccucucuuuacgcgg

SEQ ID NO: 114 - RNA, 19 nt, Artificial Sequence
  Synthetic
ccgcguaaagagaggugcg

SEQ ID NO: 115 - RNA, 19 nt, Artificial Sequence
  Synthetic
cugccgauccauacugcgg

SEQ ID NO: 116 - RNA, 19 nt, Artificial Sequence
  Synthetic
ccgcaguauggaucggcag

SEQ ID NO: 117 - RNA, 19 nt, Artificial Sequence
  Synthetic
cgugguggacuucucucaa

SEQ ID NO: 118 - RNA, 19 nt, Artificial Sequence
  Synthetic
uugagagaaguccaccacg

SEQ ID NO: 119 - RNA, 19 nt, Artificial Sequence
  Synthetic
cugcugcuaugccucaucu

SEQ ID NO: 120 - RNA, 19 nt, Artificial Sequence
  Synthetic
agaugaggcauagcagcag

SEQ ID NO: 121 - RNA, 19 nt, Artificial Sequence
  Synthetic
ccugcugcuaugccucauc

SEQ ID NO: 122 - RNA, 19 nt, Artificial Sequence
  Synthetic
gaugaggcauagcagcagg

SEQ ID NO: 123 - RNA, 19 nt, Artificial Sequence
  Synthetic
cuagacucgugguggacuu

SEQ ID NO: 124 - RNA, 19 nt, Artificial Sequence
  Synthetic
aaguccaccacgagucuag

SEQ ID NO: 125 - RNA, 19 nt, Artificial Sequence
  Synthetic
uccugcugcuaugccucau

SEQ ID NO: 126 - RNA, 19 nt, Artificial Sequence
  Synthetic
augaggcauagcagcagga

SEQ ID NO: 127 - RNA, 19 nt, Artificial Sequence
  Synthetic
gacucgugguggacuucuc

SEQ ID NO: 128 - RNA, 19 nt, Artificial Sequence
  Synthetic
gagaaguccaccacgaguc

SEQ ID NO: 129 - RNA, 19 nt, Artificial Sequence
  Synthetic
auccauacugcggaacucc

SEQ ID NO: 130 - RNA, 19 nt, Artificial Sequence
  Synthetic
ggaguuccgcaguauggau

SEQ ID NO: 131 - RNA, 19 nt, Artificial Sequence
  Synthetic
cucugccgauccauacugc

SEQ ID NO: 132 - RNA, 19 nt, Artificial Sequence
  Synthetic
gcaguauggaucggcagag

SEQ ID NO: 133 - RNA, 19 nt, Artificial Sequence
  Synthetic
gauccauacugcggaacuc

SEQ ID NO: 134 - RNA, 19 nt, Artificial Sequence
  Synthetic
gaguuccgcaguauggauc

SEQ ID NO: 135 - RNA, 19 nt, Artificial Sequence
  Synthetic
gaagaacucccucgccucg

SEQ ID NO: 136 - RNA, 19 nt, Artificial Sequence
  Synthetic
cgaggcgagggaguucuuc

SEQ ID NO: 137 - RNA, 19 nt, Artificial Sequence
  Synthetic
aagccuccaagcugugccu

SEQ ID NO: 138 - RNA, 19 nt, Artificial Sequence
  Synthetic
aggcacagcuuggaggcuu

SEQ ID NO: 139 - RNA, 19 nt, Artificial Sequence
  Synthetic
agaagaacucccucgccuc

SEQ ID NO: 140 - RNA, 19 nt, Artificial Sequence
  Synthetic
gaggcgagggaguucuucu

SEQ ID NO: 141 - RNA, 19 nt, Artificial Sequence
  Synthetic
ggaguguggauucgcacuc

SEQ ID NO: 142 - RNA, 19 nt, Artificial Sequence
  Synthetic
gagugcgaauccacacucc

SEQ ID NO: 143 - RNA, 19 nt, Artificial Sequence
  Synthetic
ccucugccgauccauacug

SEQ ID NO: 144 - RNA, 19 nt, Artificial Sequence
  Synthetic
caguauggaucggcagagg

SEQ ID NO: 145 - RNA, 19 nt, Artificial Sequence
  Synthetic
caagccuccaagcugugcc

SEQ ID NO: 146 - RNA, 19 nt, Artificial Sequence
  Synthetic
ggcacagcuuggaggcuug

SEQ ID NO: 147 - RNA, 19 nt, Artificial Sequence
  Synthetic
uccauacugcggaacuccu

SEQ ID NO: 148 - RNA, 19 nt, Artificial Sequence
  Synthetic
aggaguuccgcaguaugga

SEQ ID NO: 149 - RNA, 19 nt, Artificial Sequence
  Synthetic
cagagucuagacucguggu

SEQ ID NO: 150 - RNA, 19 nt, Artificial Sequence
  Synthetic
accacgagucuagacucug

SEQ ID NO: 151 - RNA, 19 nt, Artificial Sequence
  Synthetic
aagaagaacucccucgccu

SEQ ID NO: 152 - RNA, 19 nt, Artificial Sequence
  Synthetic
aggcgagggaguucuucuu

SEQ ID NO: 153 - RNA, 19 nt, Artificial Sequence
  Synthetic
gaguguggauucgcacucc

SEQ ID NO: 154 - RNA, 19 nt, Artificial Sequence
  Synthetic
ggagugcgaauccacacuc

SEQ ID NO: 155 - RNA, 20 nt, Artificial Sequence
  Synthetic
ucuagacucgugguggacum

SEQ ID NO: 156 - RNA, 19 nt, Artificial Sequence
  Synthetic
aguccaccacgagucuaga

SEQ ID NO: 157 - RNA, 19 nt, Artificial Sequence
  Synthetic
gcugcuaugccucaucuuc

SEQ ID NO: 158 - RNA, 19 nt, Artificial Sequence
  Synthetic
gaagaugaggcauagcagc

SEQ ID NO: 159 - RNA, 19 nt, Artificial Sequence
  Synthetic
agucuagacucguggugga

SEQ ID NO: 160 - RNA, 19 nt, Artificial Sequence
  Synthetic
uccaccacgagucuagacu

SEQ ID NO: 161 - RNA, 19 nt, Artificial Sequence
  Synthetic
cuccucugccgauccauac

SEQ ID NO: 162 - RNA, 19 nt, Artificial Sequence
  Synthetic
guauggaucggcagaggag

SEQ ID NO: 163 - RNA, 19 nt, Artificial Sequence
  Synthetic
uggcucaguuuacuagugc

SEQ ID NO: 164 - RNA, 19 nt, Artificial Sequence
  Synthetic
gcacuaguaaacugagcca

SEQ ID NO: 165 - RNA, 19 nt, Artificial Sequence
  Synthetic
gucuagacucgugguggac

SEQ ID NO: 166 - RNA, 19 nt, Artificial Sequence
  Synthetic
guccaccacgagucuagac

SEQ ID NO: 167 - RNA, 19 nt, Artificial Sequence
  Synthetic
uucaagccuccaagcugug

SEQ ID NO: 168 - RNA, 19 nt, Artificial Sequence
  Synthetic
cacagcuuggaggcuugaa

SEQ ID NO: 169 - RNA, 19 nt, Artificial Sequence
  Synthetic
cuaugggagugggccucag

SEQ ID NO: 170 - RNA, 19 nt, Artificial Sequence
  Synthetic
cugaggcccacucccauag

SEQ ID NO: 171 - RNA, 19 nt, Artificial Sequence
  Synthetic
cucgugguggacuucucuc

SEQ ID NO: 172 - RNA, 19 nt, Artificial Sequence
  Synthetic
gagagaaguccaccacgag

SEQ ID NO: 173 - RNA, 19 nt, Artificial Sequence
  Synthetic
ccuaugggagugggccuca

SEQ ID NO: 174 - RNA, 19 nt, Artificial Sequence
  Synthetic
ugaggcccacucccauagg

SEQ ID NO: 175 - RNA, 19 nt, Artificial Sequence
  Synthetic
aagaacucccucgccucgc

SEQ ID NO: 176 - RNA, 19 nt, Artificial Sequence
  Synthetic
gcgaggcgagggaguucuu

SEQ ID NO: 177 - RNA, 19 nt, Artificial Sequence
  Synthetic
ucugccgauccauacugcg

SEQ ID NO: 178 - RNA, 19 nt, Artificial Sequence
  Synthetic
cgcaguauggaucggcaga

SEQ ID NO: 179 - RNA, 19 nt, Artificial Sequence
  Synthetic
agagucuagacucguggug

SEQ ID NO: 180 - RNA, 19 nt, Artificial Sequence
  Synthetic
caccacgagucuagacucu

SEQ ID NO: 181 - RNA, 19 nt, Artificial Sequence
  Synthetic
gaagaagaacucccucgcc

SEQ ID NO: 182 - RNA, 19 nt, Artificial Sequence
  Synthetic
ggcgagggaguucuucuuc

SEQ ID NO: 183 - RNA, 19 nt, Artificial Sequence
  Synthetic
ucaagccuccaagcugugc

SEQ ID NO: 184 - RNA, 19 nt, Artificial Sequence
  Synthetic
gcacagcuuggaggcuuga

SEQ ID NO: 185 - RNA, 19 nt, Artificial Sequence
  Synthetic
agccuccaagcugugccuu

SEQ ID NO: 186 - RNA, 19 nt, Artificial Sequence
  Synthetic
aaggcacagcuuggaggcu

SEQ ID NO: 187 - RNA, 19 nt, Artificial Sequence
  Synthetic
agacucgugguggacuucu

SEQ ID NO: 188 - RNA, 19 nt, Artificial Sequence
  Synthetic
agaaguccaccacgagucu

SEQ ID NO: 189 - RNA, 19 nt, Artificial Sequence
  Synthetic
gugugcacuucgcuucaca

SEQ ID NO: 190 - RNA, 21 nt, Artificial Sequence
  Synthetic
ugugaagcgaagugcacacuu

SEQ ID NO: 191 - RNA, 21 nt, Artificial Sequence
  Synthetic
caccaugcaacuuuuucaccu

SEQ ID NO: 192 - RNA, 23 nt, Artificial Sequence
  Synthetic
aggugaaaaaguugcaugguguu

SEQ ID NO: 193 - RNA, 19 nt, Artificial Sequence
  Synthetic
auccauacugcggaacucc

SEQ ID NO: 194 - RNA, 19 nt, Artificial Sequence
  Synthetic
ggaguuccgcaguauggau

SEQ ID NO: 195 - RNA, 19 nt, Artificial Sequence
  Synthetic
cucugccgauccauacugc

SEQ ID NO: 196 - RNA, 19 nt, Artificial Sequence
  Synthetic
gcaguauggaucggcagag

SEQ ID NO: 197 - RNA, 19 nt, Artificial Sequence
  Synthetic
gauccauacugcggaacuc

SEQ ID NO: 198 - RNA, 19 nt, Artificial Sequence
  Synthetic
gaguuccgcaguauggauc

SEQ ID NO: 199 - RNA, 19 nt, Artificial Sequence
  Synthetic
gaagaacucccucgccucg

SEQ ID NO: 200 - RNA, 19 nt, Artificial Sequence
  Synthetic
cgaggcgagggaguucuuc

SEQ ID NO: 201 - RNA, 19 nt, Artificial Sequence
  Synthetic
aagccuccaagcugugccu

SEQ ID NO: 202 - RNA, 19 nt, Artificial Sequence
  Synthetic
aggcacagcuuggaggcuu

SEQ ID NO: 203 - RNA, 19 nt, Artificial Sequence
  Synthetic
agaagaacucccucgccuc

SEQ ID NO: 204 - RNA, 19 nt, Artificial Sequence
  Synthetic
gaggcgagggaguucuucu

SEQ ID NO: 205 - RNA, 19 nt, Artificial Sequence
  Synthetic
ggaguguggauucgcacuc

SEQ ID NO: 206 - RNA, 19 nt, Artificial Sequence
  Synthetic
gagugcgaauccacacucc

SEQ ID NO: 207 - RNA, 19 nt, Artificial Sequence
  Synthetic
ccucugccgauccauacug

SEQ ID NO: 208 - RNA, 19 nt, Artificial Sequence
  Synthetic
caguauggaucggcagagg

SEQ ID NO: 209 - RNA, 19 nt, Artificial Sequence
  Synthetic
caagccuccaagcugugcc

SEQ ID NO: 210 - RNA, 19 nt, Artificial Sequence
  Synthetic
ggcacagcuuggaggcuug

SEQ ID NO: 211 - RNA, 19 nt, Artificial Sequence
  Synthetic
uccauacugcggaacuccu

SEQ ID NO: 212 - RNA, 19 nt, Artificial Sequence
  Synthetic
aggaguuccgcaguaugga

SEQ ID NO: 213 - RNA, 19 nt, Artificial Sequence
  Synthetic
cagagucuagacucguggu

SEQ ID NO: 214 - RNA, 19 nt, Artificial Sequence
  Synthetic
accacgagucuagacucug

SEQ ID NO: 215 - RNA, 19 nt, Artificial Sequence
  Synthetic
aagaagaacucccucgccu

SEQ ID NO: 216 - RNA, 19 nt, Artificial Sequence
  Synthetic
aggcgagggaguucuucuu

SEQ ID NO: 217 - RNA, 19 nt, Artificial Sequence
  Synthetic
gaguguggauucgcacucc

SEQ ID NO: 218 - RNA, 19 nt, Artificial Sequence
  Synthetic
ggagugcgaauccacacuc

SEQ ID NO: 219 - RNA, 19 nt, Artificial Sequence
  Synthetic
ucuagacucgugguggacu

SEQ ID NO: 220 - RNA, 19 nt, Artificial Sequence
  Synthetic
aguccaccacgagucuaga

SEQ ID NO: 221 - RNA, 19 nt, Artificial Sequence
  Synthetic
gcugcuaugccucaucuuc

SEQ ID NO: 222 - RNA, 19 nt, Artificial Sequence
  Synthetic
gaagaugaggcauagcagc

SEQ ID NO: 223 - RNA, 19 nt, Artificial Sequence
  Synthetic
agucuagacucguggugga

SEQ ID NO: 224 - RNA, 19 nt, Artificial Sequence
  Synthetic
uccaccacgagucuagacu

SEQ ID NO: 225 - RNA, 19 nt, Artificial Sequence
  Synthetic
cuccucugccgauccauac

SEQ ID NO: 226 - RNA, 19 nt, Artificial Sequence
  Synthetic
guauggaucggcagaggag

SEQ ID NO: 227 - RNA, 19 nt, Artificial Sequence
  Synthetic
uggcucaguuuacuagugc

SEQ ID NO: 228 - RNA, 19 nt, Artificial Sequence
  Synthetic
gcacuaguaaacugagcca

SEQ ID NO: 229 - RNA, 19 nt, Artificial Sequence
  Synthetic
gucuagacucgugguggac

SEQ ID NO: 230 - RNA, 19 nt, Artificial Sequence
  Synthetic
guccaccacgagucuagac

SEQ ID NO: 231 - RNA, 19 nt, Artificial Sequence
  Synthetic
uucaagccuccaagcugug

SEQ ID NO: 232 - RNA, 19 nt, Artificial Sequence
  Synthetic
cacagcuuggaggcuugaa

SEQ ID NO: 233 - RNA, 19 nt, Artificial Sequence
  Synthetic
cuaugggagugggccucag

SEQ ID NO: 234 - RNA, 19 nt, Artificial Sequence
  Synthetic
cugaggcccacucccauag

SEQ ID NO: 235 - RNA, 19 nt, Artificial Sequence
  Synthetic
cucgugguggacuucucuc

SEQ ID NO: 236 - RNA, 19 nt, Artificial Sequence
  Synthetic
gagagaaguccaccacgag

SEQ ID NO: 237 - RNA, 19 nt, Artificial Sequence
  Synthetic
ccuaugggagugggccuca

SEQ ID NO: 238 - RNA, 19 nt, Artificial Sequence
  Synthetic
ugaggcccacucccauagg

SEQ ID NO: 239 - RNA, 19 nt, Artificial Sequence
  Synthetic
aagaacucccucgccucgc

SEQ ID NO: 240 - RNA, 19 nt, Artificial Sequence
  Synthetic
gcgaggcgagggaguucuu

SEQ ID NO: 241 - RNA, 19 nt, Artificial Sequence
  Synthetic
ucugccgauccauacugcg

SEQ ID NO: 242 - RNA, 19 nt, Artificial Sequence
  Synthetic
cgcaguauggaucggcaga

SEQ ID NO: 243 - RNA, 19 nt, Artificial Sequence
  Synthetic
agagucuagacucguggug

SEQ ID NO: 244 - RNA, 19 nt, Artificial Sequence
  Synthetic
caccacgagucuagacucu

SEQ ID NO: 245 - RNA, 19 nt, Artificial Sequence
  Synthetic
gaagaagaacucccucgcc

SEQ ID NO: 246 - RNA, 19 nt, Artificial Sequence
  Synthetic
ggcgagggaguucuucuuc

SEQ ID NO: 247 - RNA, 21 nt, Artificial Sequence
  Synthetic
ccgugugcacuucgcuucauu

SEQ ID NO: 248 - RNA, 21 nt, Artificial Sequence
  Synthetic
ugaagcgaagugcacacgguu

SEQ ID NO: 249 - RNA, 21 nt, Artificial Sequence
  Synthetic
cuggcucaguuuacuaguguu

SEQ ID NO: 250 - RNA, 21 nt, Artificial Sequence
  Synthetic
cacuaguaaacugagccaguu

SEQ ID NO: 251 - RNA, 21 nt, Artificial Sequence
  Synthetic
gccgauccauacugcggaauu

SEQ ID NO: 252 - RNA, 21 nt, Artificial Sequence
  Synthetic
uuccgcaguauggauccgcuu

SEQ ID NO: 253 - RNA, 21 nt, Artificial Sequence
  Synthetic
agguauguugcccguuuguuu

SEQ ID NO: 254 - RNA, 21 nt, Artificial Sequence
  Synthetic
acaaacgggcaacauaccuuu

SEQ ID NO: 255 - RNA, 21 nt, Artificial Sequence
  Synthetic
gcucaguuuacuagugccauu

SEQ ID NO: 256 - RNA, 21 nt, Artificial Sequence
  Synthetic
uggcacuaguaaacugagcuu

SEQ ID NO: 257 - RNA, 21 nt, Artificial Sequence
  Synthetic
caagguauguugcccguuuuu

SEQ ID NO: 258 - RNA, 21 nt, Artificial Sequence
  Synthetic
aaacgggcaacauaccuuguu

SEQ ID NO: 259 - RNA, 21 nt, Artificial Sequence
  Synthetic
cuguaggcauaaauugguauu

SEQ ID NO: 260 - RNA, 21 nt, Artificial Sequence
  Synthetic
uaccaauuuaugccuacaguu

SEQ ID NO: 261 - RNA, 21 nt, Artificial Sequence
  Synthetic
ucugcggcguuuuaucauauu

SEQ ID NO: 262 - RNA, 21 nt, Artificial Sequence
  Synthetic
uaugauaaaacgccgcagauu

SEQ ID NO: 263 - RNA, 22 nt, Artificial Sequence
  Synthetic
accucugccuaaucaucucuuu

SEQ ID NO: 264 - RNA, 21 nt, Artificial Sequence
  Synthetic
gagaugauuaggcagagguuu

SEQ ID NO: 265 - RNA, 21 nt, Artificial Sequence
  Synthetic
uuuacuagugccauuuguauu

SEQ ID NO: 266 - RNA, 21 nt, Artificial Sequence
  Synthetic
uacaaauggcacuaguaaauu

SEQ ID NO: 267 - RNA, 21 nt, Artificial Sequence
  Synthetic
accucugccuaaucaucuauu

SEQ ID NO: 268 - RNA, 21 nt, Artificial Sequence
  SyntheticM
uagaugauuaggcagagguuu

SEQ ID NO: 269 - RNA, 21 nt, Artificial Sequence
  Synthetic
cuguaggcauaaauuggucuu

SEQ ID NO: 270 - RNA, 21 nt, Artificial Sequence
  Synthetic
gaccaauuuaugccuacaguu

SEQ ID NO: 271 - RNA, 21 nt, Artificial Sequence
  Synthetic
ccgugugcacuucgcuucauu

SEQ ID NO: 272 - RNA, 21 nt, Artificial Sequence
  Synthetic
ugaagcgaagugcacacgguu

SEQ ID NO: 273 - DNA, 13 bp, Artificial Sequence
  Antisense Oligonucleotide
  misc_feature (1)..(13): phosphorothiate Internucleoside linkages
  misc_feature (1)..(2): LNA nucleosides, LNA C is 5-methyl C
  misc_feature (11)..(13): LNA nucleosides, LNA C is 5-methyl C
gcattggtattca